(19) United States
(12) Patent Application Publication
(10) Pub. No.: US 2021/0071189 A1
(43) Pub. Date: Mar. 11, 2021

(54) CPF1 BASED TRANSCRIPTION REGULATION SYSTEMS IN PLANTS

(71) Applicant: KWS SAAT SE & CO. KGaA, Einbeck (DE)

(72) Inventors: Mathias LABS, Frontenac, MO (US); Aaron HUMMEL, St. Louis, MO (US); Yu MEI, Maryland Heights, MO (US)

(73) Assignee: KWS SAAT SE & CO. KGaA, Einbeck (DE)

(21) Appl. No.: 16/955,937

(22) PCT Filed: Dec. 21, 2018
(86) PCT No.: PCT/EP2018/086725

§ 371 Date: Jun. 19, 2020

Related U.S. Application Data
(60) Provisional application No. 62/609,508, filed on Dec. 22, 2017.
(60) Provisional application No. 62/758,068, filed on Nov. 9, 2018.

(51) Int. Cl. C12N 15/82

(52) U.S. Cl. CPC C12N 15/8216; C12N 15/8217; C12N 15/8213

(57) ABSTRACT

The present invention relates to the targeted regulation of gene expression and more specifically to synthetic transcription factors (STFs) comprising at least one highly target specific engineered recognition domain based on a CRISPR/Cpf1 system and further comprising at least one activation or silencing domain to modulate the expression of a gene of interest, preferably to modulate the transcription of a morphogenic gene of a eukaryote, in particular a plant. Further disclosed are methods using the STFs to enhance transformation frequencies, to optimize successful genome editing approaches, to provide haploid or double haploid organisms, and/or to provide compositions suitable for general transformation, but also for breeding purposes.

### TECHNICAL FIELD

The present invention relates to the targeted regulation of gene expression and more specifically to synthetic transcription factors (STFs) comprising at least one highly target specific engineered recognition domain based on a CRISPR/Cpf1 system and further comprising at least one activation or silencing domain to modulate the expression of a gene of interest, preferably to modulate the transcription of a morphogenic gene of a eukaryote, in particular a plant. Further disclosed are methods using the STFs to enhance transformation frequencies, to optimize successful genome editing approaches, to provide haploid or double haploid organisms, and/or to provide compositions suitable for general transformation, but also for breeding purposes. These methods and uses rely on the synergistic interaction of the STF comprising a gene expression modulation domain, e.g. an activation domain or a silencing domain, allowing the reprogramming of a cell and the induction of cell division and/or regeneration simultaneous with transforming said cell or editing the genome of said cell.

### BACKGROUND OF THE INVENTION

The ability to efficiently transform and precisely modify genetic material in eukaryotic cells enables a wide range of high value applications in agricultural product development, basic research and other technical fields. Fundamentally, genome engineering or gene editing (GE) provides this capability by introducing predefined genetic variation at specific locations in eukaryotic as well as prokaryotic genomes. Meanwhile, there exists a plethora of methods for transforming different eukaryotic or prokaryotic cells in specific developmental stages. Still, transformation or transfection efficiencies sometimes remain very low for certain cell types or genotypes and highly specific methods fine-tuned for different cells originating from different genotypes have to be established.

Further, the ability not only to modify, but also to specifically modulate, i.e., to activate or inhibit, gene expression in a highly targeted manner has a high value in plant biotechnology.

For example, while transformation of the major monocot crops is currently possible, the process typically remains confined to one or two genotypes per species, often with poor agronomics, and efficiencies that place these methods beyond the reach of agricultural implementation.

In view of the fact that the increase of the global human population will necessitate doubling the world food production in the next few decades and at the same time climate change causes new challenges for plant breeders, there is a great need for optimized crop plants having resistance to biotic and abiotic stress, for example, resistance against emerging plant pathogens or drought resistance. Relying on classical breeding and selection technologies will likely not be effective enough to cope with the dramatically increasing demand and to establish a sustainable supply facing the eco-sociological changes in the future decades. Therefore, new strategies and biotechnological measures have to be developed to establish traits with which plants could better adapt to adverse environmental conditions.

Presently, maize is one of the most important food and feed crop as well as bio-energy source around the world. At the same time, maize has become one of the most important target crops for biotechnological innovation since the establishment of the first transgenic Bacillus thuringiensis (Bt) maize products in the mid 1990^{ies}. Despite the complexity of the maize genome (in comparison to model plants), there are meanwhile more biotech traits available on the market in maize than in any other crop plants. Transgenic maize production has made tremendous progress since the first successful report using the labor-intensive and time-consuming protoplast transformation method (Rhodes et al., 1988a). Development of microparticle bombardment transformation (Fromm et al., 1990; Gordon-Kamm et al., 1990) and Agrobacterium-mediated transformation (Ishida et al., 1996) technologies has made the generation of transgenic maize simpler and more reliable. Highly productive biolistic transformation systems were established in Hi-II with BAR as the selectable marker (Frame et al., 2000), and in the elite inbred line CG00526 with PMI as the selectable marker (Wright et al., 2001). Efficient Agrobacterium-mediated transformation systems were reported by using the inbred line A188 (Ishida et al., 1996; Negrotto et al., 2000), Hi-II (Zhao et al., 2001), and A188/Hi-II hybrids (Li et al., 2003). In the last few years, progress in genome engineering technologies has made it possible to make modifications and insert transgenes at specific chromosomal target sites in the maize genome (Shukla et al., 2009; Gao et al., 2010; Liang et al., 2014; for a review: Que et al., Front. Plant. Sci., 2014, 5, 379). Still, none of the above techniques provides reliable and transferable results applicable in different genotypes, let alone in a different plant.

Progress in the plant biotechnological field over the last decades was based on the establishment of transgenic crop plants. Socio-economic and regulatory factors, however, increasingly suggest that the development of non-transgenic plants and plant products becomes more and more important for certain countries and territories.

Morphogenesis usually means the biological process that causes an organism to develop its shape. It is one of three fundamental aspects of developmental biology along with the control of cell growth and cellular differentiation, unified in evolutionary developmental biology. An important class of molecules involved in morphogenesis are transcription factor proteins that determine the fate of cells by interacting with DNA. These can be coded for by master regulatory genes, and either activate or deactivate the transcription of other genes; in turn, these secondary gene products can regulate the expression of still other genes in a regulatory cascade of gene regulatory networks. At the end of this cascade are classes of molecules that control cellular behaviours such as cell migration, or, more generally, their properties, such as cell adhesion or cell motility, cell proliferation and apoptosis.

Recently, the group of Lowe et al. (Lowe et al., Morphogenic Regulators Baby boom and Wuschel Improve Monocot Transformation, The Plant Cell, 2016, Vol. 28: 1998-2015) reported a transformation approach involving overexpression of the maize (Zea mays) morphogenic genes Baby boom (BBM) and maize Wuschel (WUS) genes, which produced high transformation frequencies in numerous previously non-transformable maize inbred lines. Lowe et al. found out that overexpression of BBM and WUS in inbred lines which were difficult to transform, resulted in an increase in regeneration capability of transgenic calli. The role of WUS and BBM in plant development was already described earlier (U.S. Pat. No. 7,256,322 B2 or US 2013/0254935 A1).

However, the above and further approaches presently all rely on heterologous overexpression of morphogenic genes e.g. in cellular compartments where such genes are usually not expressed, or on the provision of transgenic crop plants carrying the respective genes stably incorporated in their genomes. Another strategy is the temporally or spatially regulated expression of a target gene, e.g., using inducible and/or tissue-specific promoters. Uncontrolled overexpression, however, can cause phenotypical changes that might affect the fitness and yield efficiency of crop plants making the use of such approaches in agriculture less attractive. There is thus still a great need in identifying new strategies to exploit the functions of endogenous genes, including morphogenic factors, in a targeted way avoiding the need of overexpressing heterologous genes in a cell or cellular system of interest.

Many plant cells have the ability to regenerate a complete organism from only single cells or tissues. This process is usually referred to as totipotency. This process of regeneration of a whole plant seems to be closely related to the process of morphogenesis. The capacity of in vitro cultured plant tissues and cells to undergo morphogenesis, resulting in the formation of discrete organs or even whole plants, has provided opportunities for numerous applications of in vitro plant biology in studies of basic botany, biochemistry, breeding, and development of new crop plants.

Haploids are plants that contain a gametic chromosome number (n). They can originate spontaneously in nature or as a result of various induction techniques. Spontaneous development of haploid plants has been known since 1922, when Blakeslee first described this phenomenon in Datura stramonium (Blakeslee et al., 1922); this was subsequently followed by similar reports in Nicotiana tabacum, Triticum aestivum and several other species (Forster et al., 2007). However, spontaneous occurrence of haploids is a rare event and therefore of limited practical value.

Haploids produced from diploid species, known as monoploids, contain only one set of chromosomes in the sporophytic phase. They are smaller and exhibit a lower plant vigor compared to donor plants and are sterile due to the inability of their chromosomes to pair during meiosis. In order to propagate them through seed and to include them in breeding programs, their fertility has to be restored with spontaneous or induced chromosome doubling. The obtained doubled or double haploids are homozygous at all loci and can represent a new variety (self-pollinated crops) or parental inbred line for the production of hybrid varieties (cross-pollinated crops). In fact, cross pollinated species often express a high degree of inbreeding depression. For these species, the induction process per se can serve not only as a fast method for the production of homozygous lines but also as a selection tool for the elimination of genotypes expressing strong inbreeding depression. Selection can be expected for traits caused by recessive deleterious genes that are associated with vegetative growth. Therefore, haploid and likewise double haploid plant systems are of great importance for plant breeding strategies, yet little is known about the cross-talk between developmental pathways like morphogenic pathways and a potential influence thereof in the generation of haploid plant systems.

Furthermore, there are severe problems in transforming elite germplasm carrying a highly valuable genotype, as the respective plants or plant parts or in vitro culturable cells derivable from said elite plants are usually highly recalcitrant to transformation and/or transfection. This fact makes the targeted plant development or breeding highly complicated, time-consuming and expensive, as many additional steps of breeding and/or molecular biology have to be applied to successfully transfer an elite event into a genetic background of interest.

It was therefore an aim of the present invention to develop new strategies for the induction of endogenous genes, preferably morphogenic genes, in their natural cellular environment in order to improve the regeneration of crop plants which are otherwise difficult to transform, or even highly recalcitrant to transformation/transfection by known techniques. Furthermore, it was an aim to unify the high precision available with recent gene editing technologies to provide for a tunable and adjustable approach to regulate morphogenic genes, preferably in a transient manner, to allow better transformation and regeneration capabilities in target cells or tissues without unduly influencing the endogenous morphogenesis system of a cell, wherein the approaches should be configured to allow for a genotype-independent increase in transformation/transfection rates.

Based on the exploitation of the artificial regulation of gene expression, mainly transcriptional regulation, it was another aim to provide synthetic transcription factors with silencing capacity with respect to transcriptional control to provide efficient compositions to control transcription and expression of aberrantly expressed genes.

It was a further aim to establish new strategies for providing haploid and double haploid plant cells, cellular systems and whole organisms based on the targeted modification of morphogenic genes to provide a starting material for producing double haploids for a variety of relevant crop plants, said double haploids as completely homozygous lines representing a valuable tool in plant breeding and plant biotechnology.

Transcriptional regulation tools have been developed utilizing deactivated CRISPR endonuclease fusion constructs with transcription effector domains known to activate or suppress gene transcription when recruited to promoter regions. So far, CRISPR/Cas9 based transcription activation and suppression systems have been made available for both mammalian cells and plant cell systems (Chen et al. (2013), Multiplexed activation of endogenous genes by CRISPR-on, an RNA-guided transcriptional activator system. Cell Research, 23: 1163-1171; Lowder et al. (2015), A CRISPR/Cas9 toolbox for multiplexed plant genome editing and transcriptional regulation. Plant Physiology, 169: 971-985; Lowder et al. (2017), Robust transcriptional activation in plants using multiplexed CRISPR-Act2.0 and mTALE-Act systems. Molecular Plant, 11: 245-256; and Li et al. (2017), A potent Cas9-driven gene activator for plant and animal cells. Nature Plants, 3: 930-936).

Cpf1-based transcription activation systems have several advantages over Cas9-based transcription activation systems. They can be used to target AT-rich promoter regions, whereas Cas9-based systems are specific for GC-rich regions. Because of the RNAse activity of Cpf1 being able to process multiple crRNAs from a single transcript, a Cpf1-based transcription regulation system has the advantage over commonly known Cas9-based systems, that it can be easily applied for multiplexed gene regulation.

However, Cpf1 based transcription activation systems are presently only available for mammalian cell systems (Tak et al. (2017), Inducible and multiplex gene regulation using CRISPR/Cpf1 based transcription factors. Nature Methods, 14(12):1163-1166; and Liu et al. (2017), Engineering cell signaling using tunable CRISPR/Cpf1 based transcription factors. Nature Communications, 8(1):2095), despite that Cpf1 based transcription suppression has been demonstrated in Arabidopsis (Tang et al. (2017), A CRISPR/Cpf1 system for efficient genome editing and transcriptional repression in plants. Nature Plants, 3:17018). So far, Cpf1-based transcriptional activation has not been shown in plants indicating that simple replacement of a transcription suppression domain like the one used in Tang et al. by a transcription activation domain is not possible and requires elaborate configuration and testing of the right linker and activation domain sequences. Thus, it is not known from the prior art whether the simple replacement of a suppression domain with an activation domain in a Cpf1-based system would result in the activation of endogenous gene expression. The prior art rather suggests that extensive modification and experimentation is required to provide a Cpf1-based transcriptional activator which can be used in plant cells.

In particular, it was therefore an object of the present invention to provide a Cpf1-based transcription activation (or suppression) system that can be employed in a large variety of crop plants for targeting AT-rich promoter regions, preferably of endogenous genes. The system should be easily applicable for multiplexing, i.e. to simultaneously target multiple genomic regions, by using guide RNA arrays. Furthermore, it should be possible to employ the system transiently in a transgene-free environment. In addition, it was a further aim of the present invention to establish methods to improve transformation efficiency and genome modification techniques by specifically targeting morphogenic genes for enhanced expression,

### SUMMARY OF THE INVENTION

The above objectives have been achieved by providing, in a first aspect, a synthetic transcription factor, or a nucleotide sequence encoding the same, comprising at least one recognition domain and at least one gene expression modulation domain, in particular an activation domain, wherein the synthetic transcription factor is configured to modulate the expression of a morphogenic gene in a cellular system.

Further provided is a synthetic transcription factor, wherein the at least one recognition domain is, or is a fragment of at least one disarmed CRISPR/nuclease system.

In one embodiment, there is provided a synthetic transcription factor, wherein the at least one disarmed CRISPR/nuclease system is a CRISPR/dCpf1 system, wherein the at least one disarmed CRISPR/nuclease system comprises at least one guide RNA.

In another embodiment, there is provided a synthetic transcription factor, wherein the at least one activation domain is selected from the group consisting of an acidic transcriptional activation domain, preferably, wherein the at least one activation domain is from an avirulence gene of Xanthomonas oryzae, VP16 (SEQ ID NO: 259) or tetrameric VP64 (SEQ ID NO: 260) from Herpes simplex, VPR (SEQ ID NO: 261), SAM (SEQ ID NO: 262; SEQ ID NO: 263), Scaffold (SEQ ID NO: 264; SEQ ID NO: 265), Suntag (SEQ ID NO: 266; SEQ ID NO: 267), P300 (SEQ ID NO: 268), VP160 (SEQ ID NO: 269), or any combination thereof. In a preferred embodiment of the present invention, the activation domain is VPR.

In still another embodiment, there is provided a synthetic transcription factor, wherein the at least one activation domain is located N-terminal and/or C-terminal relative to the at least one recognition domain.

In one embodiment, there is provided a synthetic transcription factor, wherein the morphogenic gene is selected from the group consisting of BBM, WUS, including WUS2, a WOX gene, a WUS or BBM homologue, Lec1, Lec2, WIND1, ESR1, PLT3, PLT5, PLT7, IPT, IPT2, Knotted1, and RKD4.

In a further embodiment, there is provided a synthetic transcription factor, wherein the morphogenic gene comprises a nucleotide sequence selected from the group consisting of (i) a nucleotide sequence set forth in any one of SEQ ID NOs: 199 to 237, (ii) a nucleotide sequence having the coding sequences of the nucleotide sequence set forth in any one of SEQ ID NOs: 199 to 237, (iii) a nucleotide sequence complementary to the nucleotide sequence of (i) or (ii), (iv) a nucleotide sequence having at least 50%, 60%, 70%, 75%, 80%, 85%, 90%, 91%, 92%, 93%, 94%, 95%, 96%, 97%, 98%, or 99% identity, preferably over the whole length, to the the nucleotide sequence of (i), (ii) or (iii), (v) a nucleotide sequence hybridzing the nucleotide sequence of (iii) under stringent conditions, (vi) a nucleotide sequence encoding a protein comprising the amino acid sequence set forth in any one of SEQ ID NOs: 238 to 258, (vii) a nucleotide sequence encoding a protein comprising the amino acid sequence at least 50%, 60%, 70%, 75%, 80%, 85%, 90%, 91%, 92%, 93%, 94%, 95%, 96%, 97%, 98%, or 99% identical to a sequence set forth in any one of SEQ ID NOs: 238 to 258, or (viii) a nucleotide sequence encoding a homologue, analogue or orthologue of protein comprising the amino acid sequence set forth in any one of SEQ ID NOs: 238 to 258.

In another embodiment, there is provided a synthetic transcription factor, wherein the synthetic transcription factor is configured to modulate expression, preferably transcription, of the morphogenic gene by binding to a regulation region located at a certain distance in relation to the start codon.

In yet another embodiment, there is provided a synthetic transcription factor, wherein the synthetic transcription factor and/or the at least one recognition domain comprises a sequence set forth in any one of SEQ ID Nos 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs 276, 277, 282, 283, 284, 288, 289, 290.

In a further embodiment, there is provided a synthetic transcription factor, wherein the cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

In one embodiment, there is provided a synthetic transcription factor, wherein the at least one part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In another embodiment, there is provided a synthetic transcription factor, wherein the at least one plant cell, the at least one plant or the at least one part of a plant originates from a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In one aspect, there is provided a method for increasing the transformation efficiency in a cellular system, wherein the method comprises the steps of: (a) providing a cellular system; (b) introducing into the cellular system at least one synthetic transcription factor, or a nucleotide sequence encoding the same; and (c) introducing into the cellular system at least one nucleotide sequence of interest; (d) optionally: culturing the cellular system under conditions to obtain a transformed progeny of the cellular system; wherein the at least one synthetic transcription factor, or the nucleotide sequence encoding the same, comprises at least one recognition domain and at least one gene expression modulation domain, in particular at least one activation domain, wherein the synthetic transcription factor is configured to modulate the expression, preferably the transcription, of at least one morphogenic gene in the cellular system; and wherein the at least one synthetic transcription factor, or the nucleotide sequence encoding the same, is introduced in parallel to, or sequentially with the introduction of the at least one nucleotide sequence of interest.

In one embodiment, there is provided a method, wherein (a) the at least one synthetic transcription factor, or the sequence encoding the same, or at least one component of the at least one synthetic transcription factor, or the sequence encoding the same; and (b) the at least one nucleotide sequence of interest is/are introduced into the cellular system by means independently selected from biological and/or physical means, including transfection, transformation, including transformation by Agrobacterium spp., preferably, Agrobacterium tumefaciens, a viral vector, biolistic bombardment, transfection using chemical agents, including polyethylene glycol transfection, electro-poration, cell fusion or any combination thereof.

In yet another embodiment, there is provided a method, wherein the at least one recognition domain is, or is a fragment of at least one disarmed CRISPR/nuclease system.

In another embodiment, there is provided a method, wherein the at least one disarmed CRISPR/nuclease system is a CRISPR/dCpf1 system, wherein the at least one disarmed CRISPR/nuclease system comprises at least one guide RNA.

In another embodiment, there is provided a method, wherein the at least one activation domain of the at least one synthetic transcription factor is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof. In a preferred embodiment of the present invention, the activation domain is VPR (SEQ ID NO: 276).

In yet another embodiment, there is provided a method, wherein the at least one activation domain of the at least one synthetic transcription factor is located N-terminal and/or C-terminal relative to the at least one recognition domain of the at least one synthetic transcription factor.

In a further embodiment, there is provided a method, wherein the at least one morphogenic gene is selected from the group consisting of BBM, WUS, including WUS2, a WOX gene, a WUS or BBM homologue, Lec1, Lec2, WIND1, ESR1, PLT3, PLT5, PLT7, IPT, IPT2, Knotted1, and RKD4.

In a further embodiment, there is provided a method, wherein the at least one morphogenic gene comprises a nucleotide sequence selected from the group consisting of (i) a nucleotide sequence set forth in any one of SEQ ID NOs: 199 to 237, (ii) a nucleotide sequence having the coding sequences of the nucleotide sequence set forth in any one of SEQ ID NOs: 199 to 237, (iii) a nucleotide sequence complementary to the nucleotide sequence of (i) or (ii), (iv) a nucleotide sequence having at least 50%, 60%, 70%, 75%, 80%, 85%, 90%, 91%, 92%, 93%, 94%, 95%, 96%, 97%, 98%, or 99% identity, preferably over the whole length, to the the nucleotide sequence of (i), (ii) or (iii), (v) a nucleotide sequence hybridzing the nucleotide sequence of (iii) under stringent conditions, (vi) a nucleotide sequence encoding a protein comprising the amino acid sequence set forth in any one of SEQ ID NOs: 238 to 258, (vii) a nucleotide sequence encoding a protein comprising the amino acid sequence at least 50%, 60%, 70%, 75%, 80%, 85%, 90%, 91%, 92%, 93%, 94%, 95%, 96%, 97%, 98%, or 99% identical to a sequence set forth in any one of SEQ ID NOs: 238 to 258, or (viii) a nucleotide sequence encoding a homologue, analogue or orthologue of protein comprising the amino acid sequence set forth in any one of SEQ ID NOs: 238 to 258.

In another embodiment, there is provided a method, wherein the synthetic transcription factor is configured to modulate expression, preferably transcription, of the morphogenic gene by binding to a regulation region located at a certain distance in relation to the start codon.

In one embodiment, there is provided a method, wherein the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID Nos: 276, 277, 282, 283, 284, 288, 289, 290.

In another embodiment, there is provided a method, wherein the cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

In a further embodiment, there is provided a method, wherein the at least one part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In yet another embodiment, there is provided a method, wherein the at least one plant cell, the at least one plant or the at least one part of a plant originates from a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In a further aspect, there is provided a method of modifying the genetic material of a cellular system at a predetermined location, wherein the method comprises the following steps: (a) providing a cellular system; (b) introducing at least one synthetic transcription factor, or a sequence encoding the same, into the cellular system, (c) further introducing into the cellular system (i) at least one site-specific nuclease, or a sequence encoding the same, wherein the site-specific nuclease induces a double-strand break at the predetermined location; (ii) optionally: at least one nucleotide sequence of interest, preferably flanked by one or more homology sequence(s) complementary to one or more nucleotide sequence(s) adjacent to the predetermined location in the genetic material of the cellular system; and; (e) optionally: determining the presence of the modification at the predetermined location in the genetic material of the cellular system; and (f) obtaining a cellular system comprising a modification at the predetermined location of the genetic material of the cellular system; wherein the at least one synthetic transcription factor, or the nucleotide sequence encoding the same, comprises at least one recognition domain and at least one activation domain, wherein the at least one synthetic transcription factor is configured to modulate the expression, preferably the transcription, of at least one morphogenic gene in the cellular system; and wherein the at least one synthetic transcription factor, or the nucleotide sequence encoding the same, is introduced in parallel to, or sequentially with the introduction of the at least one site-specific nuclease, or the sequence encoding the same and the optional at least one nucleotide sequence of interest.

In another embodiment of this aspect, there is provided a method, wherein the method further comprises the step of culturing the cellular system under conditions to obtain a genetically modified progeny of the modified cellular system.

In another embodiment of the methods of modifying the genetic material of a cellular system at a predetermined location, there is provided a method, wherein (i) the at least one synthetic transcription factor, or the sequence encoding the same, or at least one component of the at least one synthetic transcription factor, or the sequence encoding the same; and (ii) the at least one site-specific nuclease, or the sequence including the same; and optionally (iii) the at least one nucleotide sequence of interest is/are introduced into the cellular system by means independently selected from biological and/or physical means, including transfection, transformation, including transformation by Agrobacterium spp. transformation, preferably by Agrobacterium tumefaciens, a viral vector, biolistic bombardment, transfection using chemical agents, including polyethylene glycol transfection, electro-poration, cell fusion, or any combination thereof.

In one embodiment, there is provided a method, wherein the at least one recognition domain is, or is a fragment of at least one disarmed CRISPR/nuclease system.

In a further embodiment, there is provided a method, wherein the at least one disarmed CRISPR/nuclease system is a CRISPR/dCpf1 system, wherein the at least one disarmed CRISPR/nuclease system comprises at least one guide RNA.

Further provided is an embodiment of the above methods, wherein the at least one activation domain of the at least one synthetic transcription factor is selected from the group consisting of an acidic transcriptional activation domain, preferably, wherein the at least one activation domain is from a a gene of Xanthomonas oryzae, VP16 or tetrameric VP64 from Herpes simplex, VPR, SAM, Scaffold, Suntag, P300, VP160, or any combination thereof. In a preferred embodiment of the present invention, the activation domain is VPR (SEQ ID NO: 276).

In one embodiment, there is provided a method, wherein the at least one activation domain of the at least one synthetic transcription factor is located N-terminal and/or C-terminal relative to the at least one recognition domain of the at least one synthetic transcription factor.

In a further embodiment, there is provided a method, wherein the at least one morphogenic gene is selected from the group consisting of BBM, WUS, including WUS2, a WOX gene, a WUS or BBM homologue, Lec1, Lec2, WIND1, ESR1, PLT3, PLT5, PLT7, IPT, IPT2, Knotted1, and RKD4.

In a further embodiment, there is provided a method, wherein the at least one morphogenic gene comprises a nucleotide sequence selected from the group consisting of (i) a nucleotide sequence set forth in any one of SEQ ID NOs: 199 to 237, (ii) a nucleotide sequence having the coding sequences of the nucleotide sequence set forth in any one of SEQ ID NOs: 199 to 237, (iii) a nucleotide sequence complementary to the nucleotide sequence of (i) or (ii), (iv) a nucleotide sequence having at least 50%, 60%, 70%, 75%, 80%, 85%, 90%, 91%, 92%, 93%, 94%, 95%, 96%, 97%, 98%, or 99% identity, preferably over the whole length, to the the nucleotide sequence of (i), (ii) or (iii), (v) a nucleotide sequence hybridzing the nucleotide sequence of (iii) under stringent conditions, (vi) a nucleotide sequence encoding a protein comprising the amino acid sequence set forth in any one of SEQ ID NOs: 238 to 258, (vii) a nucleotide sequence encoding a protein comprising the amino acid sequence at least 50%, 60%, 70%, 75%, 80%, 85%, 90%, 91%, 92%, 93%, 94%, 95%, 96%, 97%, 98%, or 99% identical to a sequence set forth in any one of SEQ ID NOs: 238 to 258, or (viii) a nucleotide sequence encoding a homologue, analogue or orthologue of protein comprising the amino acid sequence set forth in any one of SEQ ID NOs: 238 to 258.

In another embodiment, there is provided a method, wherein the synthetic transcription factor is configured to modulate expression, preferably transcription, of the morphogenic gene by binding to a regulation region located at a certain distance in relation to the start codon.

In still another embodiment, there is provided a method, wherein the synthetic transcription factor and/or the at least one recognition domain comprises a sequence set forth in any one of SEQ ID Nos: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID Nos: 276, 277, 282, 283, 284, 288, 289, 290.

In a further embodiment, there is provided a method, wherein the cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

In yet a further embodiment, there is provided a method, wherein the one or more nucleotide sequence(s) flanking the at least one nucleotide sequence of interest at the predetermined location is/are at least 85%-100% complementary to the one or more nucleotide sequence(s) adjacent to the predetermined location, upstream and/or downstream from the predetermined location, over the entire length of the respective adjacent region(s).

In another aspect of the present invention, there is provided a method of producing a haploid or double haploid cellular system or organism, wherein the method comprises the following steps: (a) providing a haploid cellular system; (b) introducing into the haploid cellular system at least one synthetic transcription factor, or a nucleotide sequence encoding the same; (c) culturing the haploid cellular system under conditions to obtain at least one haploid or double haploid organism; and (d) optionally, selecting the at least one haploid or double haploid organism obtained in step (c), wherein the at least one synthetic transcription factor, or the nucleotide sequence encoding the same, comprises at least one recognition domain and at least one activation domain, wherein the at least one synthetic transcription factor is configured to modulate the expression, preferably the transcription, of at least one morphogenic gene in the haploid cellular system.

In one embodiment, there is provided a method, wherein the haploid cellular system of step (a) of the above method is a haploid embryo, or wherein the at least one haploid or double haploid organism of step (c) of the above method is obtained through an intermediate step of generating at least one haploid embryo from the haploid cellular system of (b).

In one embodiment, there is provided a method, wherein the at least one synthetic transcription factor, or a sequence encoding the same, or at least one component of the at least one synthetic transcription factor, or the sequence encoding the same is/are introduced into the haploid cellular system by means independently selected from biological and/or physical means, including transfection, transformation, including transformation by Agrobacterium spp. transformation, preferably by Agrobacterium tumefaciens, a viral vector, biolistic bombardment, transfection using chemical agents, including polyethylene glycol transfection, electro-poration, cell fusion, or any combination thereof.

In a further embodiment, there is provided a method, wherein the at least one recognition domain is or is a fragment of at least one disarmed CRISPR/nuclease system.

In yet a further embodiment, there is provided a method, wherein the at least one disarmed CRISPR/nuclease system is a CRISPR/dCpf1 system, wherein the at least one disarmed CRISPR/nuclease system comprises at least one guide RNA.

In another embodiment, there is provided a method, wherein the at least one activation domain of the at least one synthetic transcription factor is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof. In a preferred embodiment of the invention, the activation domain is VPR (SEQ ID NO: 276).

In a further embodiment, there is provided a method, wherein the at least one activation domain of the at least one synthetic transcription factor is located N-terminal and/or C-terminal relative to the at least one recognition domain of the at least one synthetic transcription factor.

In yet a further embodiment, there is provided a method, wherein the at least one morphogenic gene is selected from the group consisting of BBM, WUS, including WUS2, a WOX gene, a WUS or BBM homologue, Lec1, Lec2, WIND1, ESR1, PLT3, PLT5, PLT7, IPT, IPT2, Knotted1, and RKD4.

In a further embodiment, there is provided a method, wherein the at least one morphogenic gene comprises a nucleotide sequence selected from the group consisting of (i) a nucleotide sequence set forth in any one of SEQ ID NOs: 199 to 237, (ii) a nucleotide sequence having the coding sequences of the nucleotide sequence set forth in any one of SEQ ID NOs: 199 to 237, (iii) a nucleotide sequence complementary to the nucleotide sequence of (i) or (ii), (iv) a nucleotide sequence having at least 50%, 60%, 70%, 75%, 80%, 85%, 90%, 91%, 92%, 93%, 94%, 95%, 96%, 97%, 98%, or 99% identity, preferably over the whole length, to the the nucleotide sequence of (i), (ii) or (iii), (v) a nucleotide sequence hybridzing the nucleotide sequence of (iii) under stringent conditions, (vi) a nucleotide sequence encoding a protein comprising the amino acid sequence set forth in any one of SEQ ID NOs: 238 to 258, (vii) a nucleotide sequence encoding a protein comprising the amino acid sequence at least 50%, 60%, 70%, 75%, 80%, 85%, 90%, 91%, 92%, 93%, 94%, 95%, 96%, 97%, 98%, or 99% identical to a sequence set forth in any one of SEQ ID NOs: 238 to 258, or (viii) a nucleotide sequence encoding a homologue, analogue or orthologue of protein comprising the amino acid sequence set forth in any one of SEQ ID NOs: 238 to 258.

In one embodiment, there is provided a method, wherein the synthetic transcription factor is configured to modulate expression, preferably transcription, of the morphogenic gene by binding to a regulation region located at a certain distance in relation to the start codon.

In a further embodiment, there is provided a method, wherein the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

In yet a further embodiment, there is provided a method, wherein the at least one haploid cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

Further provided is cellular system or a progeny thereof obtained by any one of the methods provided herein.

In another aspect, there is provided a haploid or a double haploid cellular system or organism obtained by any one of the methods provided herein.

In another aspect, there is provided a use of a synthetic transcription factor as provided herein, or a sequence encoding the same, in any of the methods provided herein.

In a further aspect, there is provided a synthetic transcription factor, or a nucleotide sequence encoding the same, comprising at least one recognition domain and at least one activation domain, wherein the synthetic transcription factor is configured to activate the expression of an endogenous gene in a cellular system.

In yet a further aspect, there is provided a method for increasing the expression of at least one endogenous gene in a cellular system, wherein the method comprises the steps of:
(a) providing a cellular system;
(b) introducing into the cellular system at least one synthetic transcription factor, or a nucleotide sequence encoding the same;

wherein the at least one synthetic transcription factor, or the nucleotide sequence encoding the same, comprises at least one recognition domain and at least one activation domain, wherein the synthetic transcription factor is configured to increase the expression, preferably the transcription, of at least one endogenous gene in the cellular system.

Further aspects and embodiments of the present invention can be derived from the subsequent detailed description, the drawings, the sequence listing as well as the attached set of claims.

### BRIEF DESCRIPTION OF THE DRAWINGS AND SEQUENCES

FIG. 1. Illustrative examples of synthetic transcription factors (STFs) for targeted gene activation modification. (A) Targeted gene activation via TAL transcription factor is shown. TAL transcription factors consist of an activation domain (e.g. VP64) fused to the DNA-binding domain of e.g. transcription activator-like effectors (TALEs). (B) Targeted gene activation via the CRISPR/dCas9 and/or CRISPR/dCpf1 transcription system is shown. CRISPR/dCas9 and CRISPR/dCpf1 transcription factor systems comprise a disarmed nuclease (e.g. dCas9 or dCpf1) fused to an activation domain (e.g. VP64). DNA binding is mediated by a guide RNA associated with the disarmed nuclease. Upon binding to the genomic target site in close proximity to the transcription start site of a morphogenic gene of interest the STFs recruit the RNA polymerase II complex (i.e. the transcription complex) via the activation domain to the promoter region of the morphogenic gene where transcription of the gene is initiated.

FIG. 2. Schematic depiction of improved gene editing by cotransfection of a gene editing machinery with an exemplary synthetic transcription factors (STFs) specific for morphogenic genes. Modifications such as INDELs or replacement of a target gene with a repair template by a gene editing machinery (e.g. CRSPR/Cpf1 or CRSIPR/Cas9) results in genetically modified plant cell(s). Transient co-transfection of the gene editing machinery with one or more STFs specific for BBM and WUS ensure recovery of the target cell and increase of regeneration of an edited plant.

FIG. 3. Design of Tal effector binding sites targeting endogenous Wuschel (WUS) and Babyboom (BBM) genes. The sites were designed with varying distances to the start codon. (A) Binding sites for endogenous WUS (shown part thereof is set forth in SEQ ID NO: 315) are 18 base pairs in length and further comprise an initial T nucleobase (TALE 1, 2 and 3). (B) Binding sites for endogenous BBM (shown part thereof is set forth in SEQ ID NO: 316) are 24 base pairs in length and further comprises an initial T nucleobase (TALE 4, 5, and 6).

FIG. 4. Transient expression of endogenous WUS and BBM by TALE transcription factors. Induction of gene expression by TAL transcription factors was tested in a maize protoplast assay system. Maize protoplasts were transformed with vector constructs comprising TALE transcription factors targeting WUS or BBM by using a PEG-based transformation system. Experiments were performed in triplicates and repeated four times as biological replicates. After 24 hrs, cDNA was generated from extracted protoplast RNA by using commercially available kits. The expression of endogenpus WUS and BBM was determined by using a SYBR Green qRT-PCR approach. (A) The results indicate that the synthetic transcription factor TALE1 is the strongest inducer for endogenous WUS showing an average fold change of 60 in endogenous WUS gene expression. (B) The results indicate that the synthetic transcription factor TALES is the strongest inducer for endogenous BBM showing an average fold change of 490 in endogenous BBM gene expression.

FIG. 5. Evaluation of phenotypic function of endogenous ZmWUS induced by transient TALE transcription factor. In order to evaluate the effect of synthetic transcription factors on regeneration and embryogenesis, callus tissue from corn A188 was transformed by particle bombardment with the fluorescent marker tdTomato (tdT), TALE1 and PLT7. Constructs were delivered to a single cell and induction of cell proliferation was confirmed by fluorescent microscopy upon detection of the red fluorescent signal of tdT (see white circle and arrow).

FIG. 6. Plasmid map of of pGEP767 (A), pGEP761 (B) and pGEP772 (C) prepared in example 13.

FIG. 7: Guide RNA design for ZmBBM gene (A) (shown part thereof is set forth in SEQ ID NO: 317) and ZmWUS2 gene (B) (shown part thereof is set forth in SEQ ID NO: 318) in example 14. Selected TTTV, TYCV and TATV PAMs are marked with the respective arrows. Designed guide RNAs are indicated as black arrows. The ones tested in transcriptional activation are highlighted in circles.

FIG. 8: Plasmid map of pGEP667, a representative of final construct expressing a guide RNA (here: crGEP186).

FIG. 9: Transcriptional activation of WUS2 and BBM expression as determined in example 15. Using guide RNAs targeting WUS2 promoter region, the tested guides (crGEP186 and crGEP201) resulted in significant activation of WUS2 expression (A). Similarly, two guide RNAs targeting the BBM promoter region (crGEP210 and crGEP211) resulted in significant activation of BBM expression (B). Expression levels of BBM and WUS2 in samples transformed with only the LbCpf1-VPR expression vector were used as controls.

FIG. 10: Guide RNA sequences targeting ZmBBM and ZmWUS2 as designed in example 14.

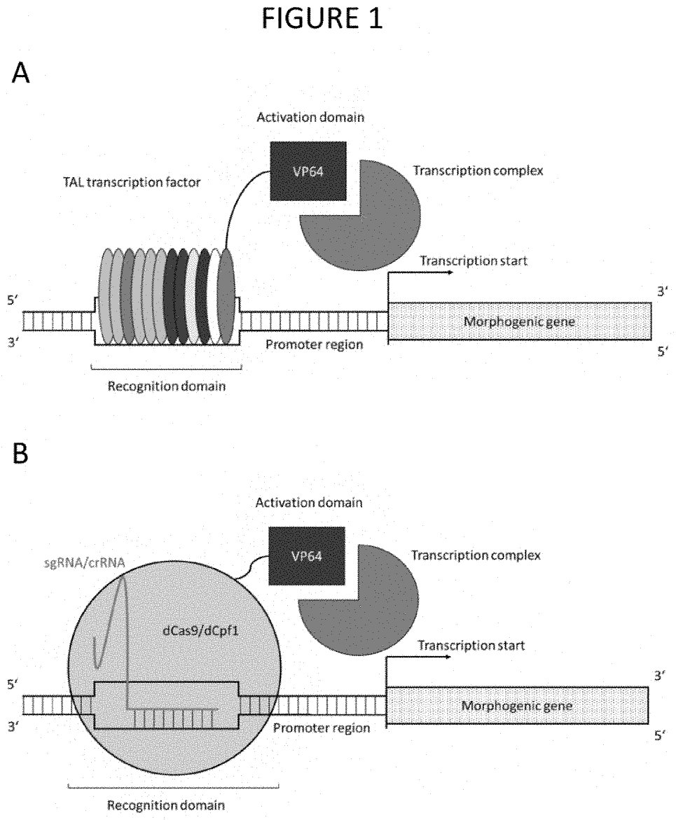

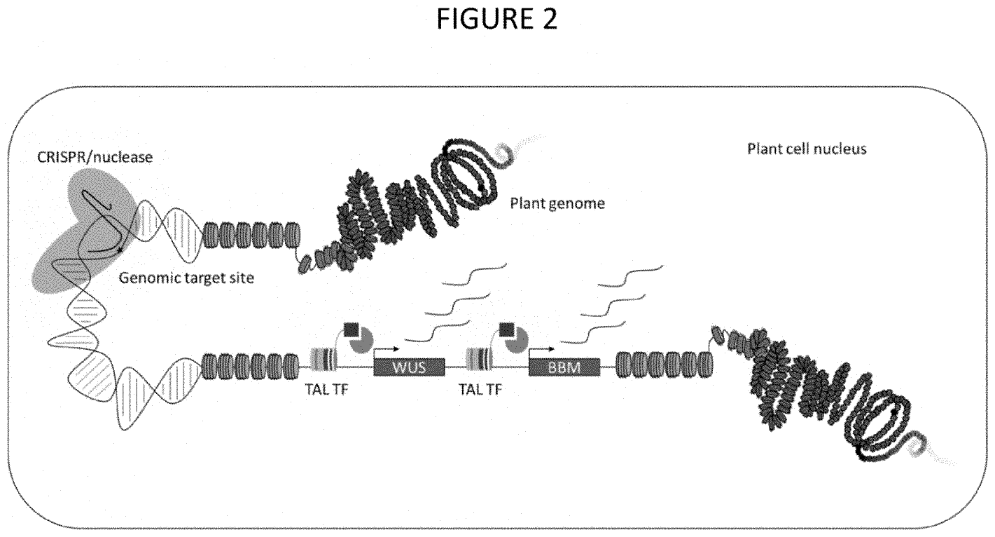

TABLE 1
Brief description of sequences disclosed in the sequence listing

| Sequence Identifier | | Sequence Identifier | |
|---|---|---|---|
| [SEQ ID NO]: | Description | [SEQ ID NO]: | description |
| 1-3 | gRNAs of Cas9 targeted to | 277 | 5xGS linker |
| | promoter region of BBM from | | |
| | Zea mays | | |
| 4-6 | gRNAs of Cas9 targeted to | | |
| | promoter region of WUS from | | |
| | Zea mays | | |
| 7-9 | crRNAs of Cpf1 targeted to | 278 | Sequence of plasmid pKWS20 |
| | promoter region of BBM from | | |
| | Zea mays | | |
| 10-12 | crRNAs of Cpf1 targeted to | | |
| | promoter region of WUS from | | |
| | Zea mays | | |
| 13-51 | TAL recognition domains | 279 | Sequence of expression |
| | targeted to promoter region of | | plasmid pGEP754 |
| | BBM from Zea mays | | |
| 52-94 | TAL recognition domains | 280 | Sequence of expression |
| | targeted to promoter region of | | plasmid pGEP755 |
| | WUS from Zea mays | | |
| 95 | Target promoter region of BBM | 281 | Sequence of expression |
| | from Zea mays | | plasmid pGEP756 |
| 96 | Target promoter region of | 282 | Wild type LbCpf1 |
| | WUS from Zea mays | | |
| 97-99 | Target sites of gRNAs of Cas9 | 283 | RR variant of LbCpf1 |
| | in promoter region of BBM from | | |
| | Zea mays | | |
| 100-102 | Target sites of crRNAs of Cpf1 | 284 | RVR variant of LbCpf1 |
| | in promoter region of BBM from | | |
| | Zea mays | | |
| 103-105 | Target sites of gRNAs of Cas9 | 285 | Sequence of expression |
| | in promoter region of WUS | | plasmid pGEP767 |
| | from Zea mays | | |
| 106-108 | Target sites of crRNAs of Cpf1 | 286 | Sequence of expression |
| | in promoter region of WUS | | plasmid pGEP772 |
| | from Zea mays | | |
| 109-147 | Target sites of TAL effector in | 287 | Sequence of expression |
| | promoter region of BBM from | | plasmid pGEP761 |
| | Zea mays | | |
| 148-190 | Target sites of TAL effector in | 288 | dLbCpf1-VPR |
| | promoter region of WUS from | | |
| | Zea mays | | |
| 191-198 | Primers | 289 | dLbCpf1(RR)-VPR |
| 199-216 | cDNAs of diverse morphogenic | 290 | dLbCpf1(RVR)-VPR |
| | genes from various species | | |
| 217-237 | cDNAs of diverse morphogenic | 291-294 | gRNAs targeting WUS2 |
| | genes from Zea mays | | |
| 238-258 | Amino acid sequences of | | |
| | diverse morphogenic genes | | |
| | from various species | | |
| 259-269 | Various exemplary nucleotide | 295-298 | gRNAs targeting BBM |
| | sequences encoding activation | | |
| | domains or parts thereof | | |
| 270-272 | BBM target sequences | | |
| 273 | Sequence of expression | 299-306 | Expression plasmids for gRNAs |
| | plasmid pGEP362 | | |
| 274 | Sequence of expression | 307 | Zea mays BBM |
| | plasmid pGEP487 | | |
| 275 | Sequence of expression | 308 | Zea mays WUS2 |
| | plasmid pGEP488 | | |
| 276 | VPR transcriptional | | |
| | activation domain | | |

### Definitions

The terms “site-specific DNA modifying enzyme”, “sequence-specific DNA modifying enzyme”, “gene editing enzyme”, “genome editing enzyme”, and “genome engineering enzyme” are used interchangeably herein and refer to enzymes or enzyme complexes used to make targeted, specific modification, or targeted, random modification of any genetic or epigenetic information or genome of a living organism at at least one position. The sequence-specific nature of the enzymes means that they can be targeted to edit genes, but also editing of regions other than gene encoding regions of a genome. It further comprises the editing or engineering of the nuclear (if present) as well as other genetic information of a cell. Furthermore, the modification of genetic information comprises the targeted modification of editing, engineering, mutating, or destroying nucleic acid bases contained within nuclear or extranuclear genomes, including either DNA or RNA genomes. It can also include the targeted modification of messages expressed from genomes, such as for example, RNA messages. Such enzymes include, but are not limited to, exonucleases, endonucleases, nickases, helicases, polymerases, ligases, and deaminases including cytidine, adenine, or other base editors. The modification of epigenetic information comprises the targeted modification of methylation, histone modification or of non-coding RNAs possibly causing heritable changes in gene expression.

A “base editor” as used herein refers to a protein or a complex comprising at least one protein or a fragment thereof having the capacity to mediate a targeted base modification, i.e., the conversion of a base of interest resulting in a point mutation of interest. Preferably, the at least one base editor in the context of the present invention comprises at least one nucleic acid recognition domain for targeting the base editor to a specific site of a nucleic acid sequence and at least one nucleic acid editing domain, which performs the conversion of at least one nucleobase at the specific target site. The nucleic acid recognition domain can additionally comprise at least one nucleic acid molecule, e.g., a guide RNA, or any other single- or double-stranded nucleic acid molecule. A “base edit” therefore refers to at least one specific nucleotide carrying a different nucleobase than previously. Based on the above, a “predetermined location” according to the present invention means the location or site in a genomic material in a cellular system, or within a genome of a cell of interest to be modified, where a targeted edit is to be introduced. The base editor may comprise further components besides the nucleic acid recognition domain and the nucleic acid editing domain, such as spacers, localization signals and components inhibiting naturally occurring DNA or RNA repair mechanisms to ensure the desired editing outcome. The term “nucleic acid recognition domain” refers to the component of the base editor, which ensures the site-specificity of the base editor by directing it to a target site within the predetermined location. A nucleic acid recognition domain may be based on a CRISPR system, which specifically recognizes a target sequence within the nucleic acid molecule of the cellular system using a guide RNA (gRNA) or single guide RNA (sgRNA), may be a synthetic fusion of a CRISPR RNA (crRNA) and a trans-activating crRNA (tracrRNA).

A “CRISPR nuclease”, as used herein, is any nuclease which has been identified in a naturally occurring CRISPR system, which has subsequently been isolated from its natural context, and which preferably has been modified or combined into a recombinant construct of interest to be suitable as tool for targeted genome engineering. Any CRISPR nuclease can be used and optionally reprogrammed or additionally mutated to be suitable for the various embodiments according to the present invention as long as the original wild-type CRISPR nuclease provides for DNA recognition, i.e., binding properties. Said DNA recognition can be PAM (pro-tospacer adjacent motif) dependent. CRISPR nucleases having optimized and engineered PAM recognition patterns can be used and created for a specific application. The expansion of the PAM recognition code can be suitable to target site-specific effector complexes to a target site of interest, independent of the original PAM specificity of the wild-type CRISPR-based nuclease. CRISPR nucleases also comprise mutants or catalytically active fragments or fusions of a naturally occurring CRISPR effector sequences, or the respective sequences encoding the same. A CRISPR nuclease may in particular also refer to a CRISPR nickase or even a nuclease-deficient variant of a CRISPR polypeptide having endonucleolytic function in its natural environment.

The term “nucleic acid editing domain” refers to the component of the base editor, which initiates the nucleotide conversion to result in the desired edit. The catalytic function of the nucleic acid editing domain may be a cytidine deaminase or an adenine deaminase function.

In general, base editors are composed of at least one nucleic acid recognition domain and at least one nucleic acid editing domain that deaminates cytidine or adenine. Nucleic acid editing domains which deaminate cytidine are able to convert C to T (G to A), and they are called BEs; nucleic acid editing domain which deaminate adenine can convert A to G (T to C), and they are called ABEs.

Base editors usually are composed of cytidine deaminase domain (such as APOBEC1, APOBEC3A, APOBEC3G, PmCDA1, AID), linker (usually XTEN), CRISPR domain (d/nCas9, dCpf1, CasX, CasY, or other suitable domains) and uracil DNA glycosylase inhibitor (UGI). In a modified system, the number of UGI domain or NLS can vary, so does the length of the linker. It can also include other domains such as Gam (e.g. in BE4). There can be variants with amino acid point mutations in the cytidine deaminase domain for different editing window, such as YE-BE3, YEE-BE3 and also mutations in the CRISPR domain for different PAM recognition, such as VQR-BE3, EQR-BE3, VRER-BE3, and SaKKH-BE3. In the BE-PLUS system, the CRISPR domain and cytidine deaminase domain is not expressed as fusion protein but instead linked together using a Suntag system for broadening the editing window. More details on preferred base editors, including cytidine deaminase-based DNA base editors, adenine deaminase-based DNA base editors, can be derived from Eid A et al. (Ayman Eid, Sahar Alshareef and Magdy M. Mahfouz (2018), CRISPR base editors: genome editing without double-strand breaks, Biochemical Journal (2018) 475 1955-1964).

The terms “associated with” or “in association with” according to the present disclosure are to be construed broadly and, therefore, according to present invention imply that a molecule (DNA, RNA, amino acid, comprising naturally occurring and/or synthetic building blocks) is provided in physical association with another molecule, the association being either of covalent or non-covalent nature. For example, a repair template can be associated with a gRNA of a CRISPR nuclease, wherein the association can be of non-covalent nature (complementary base pairing), or the molecules can be physically attached to each other by a covalent bond.

The term “catalytically active fragment” as used herein referring to amino acid sequences denotes the core sequence derived from a given template amino acid sequence, or a nucleic acid sequence encoding the same, comprising all or part of the active site of the template sequence with the proviso that the resulting catalytically active fragment still possesses the activity characterizing the template sequence, for which the active site of the native enzyme or a variant thereof is responsible. Said modifications are suitable to generate less bulky amino acid sequences still having the same activity as a template sequence making the catalytically active fragment a more versatile or more stable tool being sterically less demanding.

A “covalent attachment” or “covalent bond” is a chemical bond that involves the sharing of electron pairs between atoms of the molecules or sequences covalently attached to each other. A “non-covalent” interaction differs from a covalent bond in that it does not involve the sharing of electrons, but rather involves more dispersed variations of electromagnetic interactions between molecules/sequences or within a molecule/sequence. Non-covalent interactions or attachments thus comprise electrostatic interactions, van der Waals forces, Tr-effects and hydrophobic effects. Of special importance in the context of nucleic acid molecules are hydrogen bonds as electrostatic interaction. A hydrogen bond (H-bond) is a specific type of dipole-dipole interaction that involves the interaction between a partially positive hydrogen atom and a highly electronegative, partially negative oxygen, nitrogen, sulfur, or fluorine atom not covalently bound to said hydrogen atom. Any “association” or “physical association” as used herein thus implies a covalent or non-covalent interaction or attachment. In the case of molecular complexes, e.g. a complex formed by a CRISPR nuclease, a gRNA and a repair template (RT), more covalent and non-covalent interactions can be present for linking and thus associating the different components of a molecular complex of interest.

The terms “CRISPR polypeptide”, “CRISPR endonuclease”, “CRISPR nuclease”, “CRISPR protein”, “CRISPR effector” or “CRISPR enzyme” are used interchangeably herein and refer to any naturally occurring or artificial amino acid sequence, or the nucleic acid sequence encoding the same, acting as site-specific DNA nuclease or nickase, wherein the “CRISPR polypeptide” is derived from a CRISPR system of any organism, which can be cloned and used for targeted genome engineering. The terms “CRISPR nuclease” or “CRISPR polypeptide” also comprise mutants or catalytically active fragments or fusions of a naturally occurring CRISPR effector sequences, or the respective sequences encoding the same. A “CRISPR nuclease” or “CRISPR polypeptide” may thus, for example, also refer to a CRISPR nickase or even a nuclease-deficient variant of a CRISPR polypeptide having endonucleolytic function in its natural environment. Preferably, the disclosure of the present invention relies on nuclease-deficient CRISPR nucleases, still possessing their inherent DNA recognition and binding properties assisted by a cognate CRISPR RNA.

Nucleic acid sequences disclosed herein may be “codon-optimized”. “Codon optimization” implies that a DNA or RNA synthetically produced or isolated from a donor organism is adapted to the codon usage of different acceptor organism to improve transcription rates, mRNA processing and/or stability, and/or translation rates, and/or subsequent protein folding of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism. In turn, nucleic acid sequences as defined herein may have a certain degree of identity to a different sequence, encoding the same protein, but having been codon optimized.

“Complementary” or “complementarity” as used herein describes the relationship between two (c)DNA, two RNA, or between an RNA and a (c)DNA nucleic acid region. Defined by the nucleobases of the DNA or RNA, two nucleic acid regions can hybridize to each other in accordance with the lock-and-key model. To this end the principles of Watson-Crick base pairing have the basis adenine and thymine/uracil as well as guanine and cytosine, respectively, as complementary bases apply. Furthermore, also non-Watson-Crick pairing, like reverse-Watson-Crick, Hoogsteen, reverse-Hoogsteen and Wobble pairing are comprised by the term “complementary” as used herein as long as the respective base pairs can build hydrogen bonding to each other, i.e. two different nucleic acid strands can hybridize to each other based on said complementarity.

As used in the context of the present application, the term “about” can mean+/−10% of the recited value, preferably +/−5% of the recited value. For example, about 100 nucleotides (nt) shall then be understood as a value between 90 and 110 nt, preferably between 95 and 105.

The term “derivative” or “descendant” or “progeny” as used herein in the context of a prokaryotic or a eukaryotic cell, preferably an animal cell and more preferably a plant or plant cell or plant material according to the present disclosure relates to the descendants of such a cell or material which result from natural reproductive propagation including sexual and asexual propagation. It is well known to the person having skill in the art that said propagation can lead to the introduction of mutations into the genome of an organism resulting from natural phenomena which results in a descendant or progeny, which is genomically different to the parental organism or cell, however, still belongs to the same genus/species and possesses mostly the same characteristics as the parental recombinant host cell. Such derivatives or descendants or progeny resulting from natural phenomena during reproduction or regeneration are thus comprised by the term of the present disclosure and can be readily identified by the skilled person when comparing the “derivative” or “descendant” or “progeny” to the respective parent or ancestor. Furthermore, the term “derivative”, in the context of a substance or nucleic acid or amino acid molecule and not referring to a replicating cell or organism, can imply a substance or molecule derived from the original substance or molecule by chemical and/or biotechnological means. The resulting derivative will have characteristics allowing the skilled person to clearly define the original or parent molecule the derivative stems from. Furthermore, the derivative might have additional or varying biological functionalities, still a derivative or an “active fragment” of an original molecule will still share at least one biological function of the parent molecule, even though the derivative or active fragment might be shorter/longer than the parent sequence and might comprise certain mutations, deletions or insertions in comparison to the respective parent sequence.

A “eukaryotic cell” as used herein refers to a cell having a true nucleus, a nuclear membrane and organelles belonging to any one of the kingdoms of Protista, Plantae, Fungi, or Animalia. Eukaryotic organisms can comprise monocellular and multicellular organisms. Preferred eukaryotic cells and organisms according to the present invention are plant cells.

As used herein, “fusion” can refer to a protein and/or nucleic acid comprising one or more non-native sequences (e.g., moieties). Any nucleic acid sequence or amino acid sequence according to the present invention can thus be provided in the form of a fusion molecule. A fusion can be at the N-terminal or C-terminal end of the modified protein, or both, or within the molecule as separate domain. For nucleic acid molecules, the fusion molecule can be attached at the 5′ or 3′ end, or at any suitable position in between. A fusion can be a transcriptional and/or translational fusion. A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the at least one synthetic transcription factor as disclosed herein (e.g., a nuclear localization signal (NLS) for targeting (e.g., a site-specific nuclease) to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as alexa fluor dyes, Cyanine3 dye, Cyanine5 dye. The fusion can provide for increased or decreased stability. In some embodiments, a fusion can comprise a detectable label, including a moiety that can provide a detectable signal. Suitable detectable labels and/or moieties that can provide a detectable signal can include, but are not limited to, an enzyme, a radioisotope, a member of a specific binding pair; a fluorophore; a fluorescent reporter or fluorescent protein; a quantum dot; and the like. A fusion can comprise a member of a FRET pair, or a fluorophore/quantum dot donor/acceptor pair. A fusion can comprise an enzyme. Suitable enzymes can include, but are not limited to, horse radish peroxidase, luciferase, beta-25 galactosidase, and the like. A fusion can comprise a fluorescent protein. Suitable fluorescent proteins can include, but are not limited to, a green fluorescent protein (GFP), (e.g., a GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum) any of a variety of fluorescent and colored proteins. A fusion can comprise a nanoparticle. Suitable nanoparticles can include fluorescent or luminescent nanoparticles, and magnetic nanoparticles, or nanodiamonds, optionally linked to a nanoparticle. Any optical or magnetic property or characteristic of the nanoparticle(s) can be detected. A fusion can comprise a helicase, a nuclease (e.g., FokI), an endonuclease, an exonuclease (e.g., a 5′ exonuclease and/or 3′ exonuclease), a ligase, a nickase, a nuclease-helicase (e.g., Cas3), a DNA methyltransferase (e.g., Dam), or DNA demethylase, a histone methyltransferase, a histone demethylase, an acetylase (including for example and not limitation, a histone acetylase), a deacetylase (including for example and not limitation, a histone deacetylase), a phosphatase, a kinase, a transcription (co-) activator, a transcription (co-) factor, an RNA polymerase subunit, a transcription repressor, a DNA binding protein, a DNA structuring protein, a long non-coding RNA, a DNA repair protein (e.g., a protein involved in repair of either single- and/or double-stranded breaks, e.g., proteins involved in base excision repair, nucleotide excision repair, mismatch repair, NHEJ, HR, microhomology-mediated end joining (MMEJ), and/or alternative non-homologous end-joining (ANHEJ), such as for example and not limitation, HR regulators and HR complex assembly signals), a marker protein, a reporter protein, a fluorescent protein, a ligand binding protein (e.g., mCherry or a heavy metal binding protein), a signal peptide (e.g., Tat-signal sequence), a targeting protein or peptide, a subcellular localization sequence (e.g., nuclear localization sequence, a chloroplast localization sequence), and/or an antibody epitope, or any combination thereof.

A “gene” as used herein refers to a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The term “gene expression” or “expression” as used herein refers to the conversion of the information, contained in a gene, into a “gene product”. A “gene product” can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

The term “gene activation” or “augmentation/augmenting/activating/upregulating (of) gene expression” refer to any process which results in an increase in production of a gene product. A gene product can be either RNA (including, but not limited to, mRNA, rRNA, tRNA, and structural RNA) or a protein. Accordingly, gene activation includes those processes which increase transcription of a gene and/or translation of an mRNA. Examples of gene activation processes which increase transcription include, but are not limited to, those which facilitate formation of a transcription initiation complex, those which increase transcription initiation rate, those which increase transcription elongation rate, those which increase processivity of transcription and those which relieve transcriptional repression (by, for example, blocking the binding of a transcriptional repressor). Gene activation can constitute, for example, inhibition of repression as well as stimulation of expression above an existing level. Examples of gene activation processes which increase translation include those which increase translational initiation, those which increase translational elongation and those which increase mRNA stability. In general, gene activation comprises any detectable increase in the production of a gene product, preferably an increase in production of a gene product by about 2-fold, more preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100-fold or any integral value therebetween, more preferably 100-fold or more.

In contrast, the terms “gene repression” or “inhibition/inhibiting/repressing/silencing/downregulating (of) gene expression” refer to any process which results in a decrease in production of a gene product. A gene product can be either RNA (including, but not limited to, mRNA, rRNA, tRNA, and structural RNA) or protein. Accordingly, gene repression includes those processes which decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes which decrease translation include those which decrease translational initiation, those which decrease translational elongation and those which decrease mRNA stability. Transcriptional repression includes both reversible and irreversible inactivation of gene transcription. In general, gene repression comprises any detectable decrease in the production of a gene product, preferably a decrease in production of a gene product by about 2-fold, more preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100 fold or any integral value therebetween, more preferably 100-fold or more. Most preferably, gene repression results in complete inhibition of gene expression, such that no gene product is detectable.

The terms “genetic construct” or “recombinant construct”, “vector”, or “plasmid (vector)” (e.g., in the context of at least one nucleic acid sequence to be introduced into a cellular system) are used herein to refer to a construct comprising, inter alia, plasmids or (plasmid) vectors, cosmids, artificial yeast- or bacterial artificial chromosomes (YACs and BACs), phagemides, bacterial phage based vectors, an expression cassette, isolated single-stranded or double-stranded nucleic acid sequences, comprising DNA and RNA sequences in linear or circular form, or amino acid sequences, viral vectors, including modified viruses, and a combination or a mixture thereof, for introduction or transformation, transfection or transduction into any prokaryotic or eukaryotic target cell, including a plant, plant cell, tissue, organ or material according to the present disclosure. “Recombinant” in the context of a biological material, e.g., a cell or vector, thus implies an artificially produced material. A recombinant construct according to the present disclosure can comprise an effector domain, either in the form of a nucleic acid or an amino acid sequence, wherein an effector domain represents a molecule, which can exert an effect in a target cell and includes a transgene, an single-stranded or double-stranded RNA molecule, including a guide RNA ((s)gRNA), a miRNA or an siRNA, or an amino acid sequences, including, inter alia, an enzyme or a catalytically active fragment thereof, a binding protein, an antibody, a transcription factor, a nuclease, preferably a site specific nuclease, and the like. Furthermore, the recombinant construct can comprise regulatory sequences and/or localization sequences. The recombinant construct can be integrated into a vector, including a plasmid vector, and/or it can be present isolated from a vector structure, for example, in the form of a polypeptide sequence or as a non-vector connected single-stranded or double-stranded nucleic acid. After its introduction, e.g. by transformation or transfection by biological or physical means, the genetic construct can either persist extrachromosomally, i.e. non-integrated into the genome of the target cell, for example in the form of a double-stranded or single-stranded DNA, a double-stranded or single-stranded RNA or as an amino acid sequence. Alternatively, the genetic construct, or parts thereof, according to the present disclosure can be stably integrated into the genome of a target cell, including the nuclear genome or further genetic elements of a target cell, including the genome of plastids like mitochondria or chloroplasts. The term plasmid vector as used in this connection refers to a genetic construct originally obtained from a plasmid. A plasmid usually refers to a circular autonomously replicating extrachromosomal element in the form of a double-stranded nucleic acid sequence. In the field of genetic engineering these plasmids are routinely subjected to targeted modifications by inserting, for example, genes encoding a resistance against an antibiotic or an herbicide, a gene encoding a target nucleic acid sequence, a localization sequence, a regulatory sequence, a tag sequence, a marker gene, including an antibiotic marker or a fluorescent marker, a sequence, optionally encoding, a readily identifiable and the like. The structural components of the original plasmid, like the origin of replication, are maintained. According to certain embodiments of the present invention, the localization sequence can comprise a nuclear localization sequence (NLS), a plastid localization sequence, preferably a mitochondrion localization sequence or a chloroplast localization sequence. Said localization sequences are available to the skilled person in the field of plant biotechnology. A variety of plasmid vectors for use in different target cells of interest is commercially available and the modification thereof is known to the skilled person in the respective field.

A “genome” as used herein includes both the genes (the coding regions), the non-coding DNA and, if present, the genetic material of the mitochondria and/or chloroplasts, or the genomic material encoding a virus, or part of a virus. The “genome” or “genetic material” of an organism usually consists of DNA, wherein the genome of a virus may consist of RNA (single-stranded or double-stranded).

The terms “genome editing”, “gene editing” and “genome engineering” are used interchangeably herein and refer to strategies and techniques for the targeted, specific modification of any genetic information or genome of a living organism at at least one position. As such, the terms comprise gene editing, but also the editing of regions other than gene encoding regions of a genome. It further comprises the editing or engineering of the nuclear (if present) as well as other genetic information of a cell. Furthermore, the terms “genome editing”, “gene editing” and “genome engineering” also comprise an epigenetic editing or engineering, i.e. the targeted modification of, e.g. methylation, histone modification or of non-coding RNAs possibly causing heritable changes in gene expression.

“Germplasm”, as used herein, is a term used to describe the genetic resources, or more precisely the DNA of an organism and collections of that material. In breeding technology, the term germplasm is used to indicate the collection of genetic material from which a new plant or plant variety can be created.

The terms “guide RNA”, “gRNA”, “CRISPR nucleic acid sequence”, “single guide RNA”, or “sgRNA” are used interchangeably herein and either refer to a synthetic fusion of a CRISPR RNA (crRNA) and a trans-activating crRNA (tracrRNA), or the term refers to a single RNA molecule consisting only of a crRNA and/or a tracrRNA, or the term refers to a gRNA individually comprising a crRNA or a tracrRNA moiety. A tracr and a crRNA moiety, if present as required by the respective CRISPR polypeptide, thus do not necessarily have to be present on one covalently attached RNA molecule, yet they can also be comprised by two individual RNA molecules, which can associate or can be associated by non-covalent or covalent interaction to provide a gRNA according to the present disclosure. In the case of single RNA-guided endonucleases like Cpf1 (see Zetsche et al., 2015), for example, a crRNA as single guide nucleic acid sequence might be sufficient for mediating DNA targeting.

The term “hybridization” as used herein refers to the pairing of complementary nucleic acids, i.e., DNA and/or RNA, using any process by which a strand of nucleic acid joins with a complementary strand through base pairing to form a hybridized complex. Hybridization and the strength of hybridization (i.e., the strength of the association between the nucleic acids) is impacted by such factors as the degree and length of complementarity between the nucleic acids, stringency of the conditions involved, the Tm of the formed hybrid, and the G:C ratio within the nucleic acids. The term hybridized complex refers to a complex formed between two nucleic acid sequences by virtue of the formation of hydrogen bounds between complementary G and C bases and between complementary A and T/U bases. A hybridized complex or a corresponding hybrid construct can be formed between two DNA nucleic acid molecules, between two RNA nucleic acid molecules or between a DNA and an RNA nucleic acid molecule. For all constellations, the nucleic acid molecules can be naturally occurring nucleic acid molecules generated in vitro or in vivo and/or artificial or synthetic nucleic acid molecules. Hybridization as detailed above, e.g., Watson-Crick base pairs, which can form between DNA, RNA and DNA/RNA sequences, are dictated by a specific hydrogen bonding pattern, which thus represents a non-covalent attachment form according to the present invention. In the context of hybridization, the term “stringent hybridization conditions” should be understood to mean those conditions under which a hybridization takes place primarily only between homologous nucleic acid molecules. The term “hybridization conditions” in this respect refers not only to the actual conditions prevailing during actual agglomeration of the nucleic acids, but also to the conditions prevailing during the subsequent washing steps. Examples of stringent hybridization conditions are conditions under which primarily only those nucleic acid molecules that have at least 70%, preferably at least 75%, at least 80%, at least 85%, at least 90%, at least 91%, at least 92%, at least 93%, at least 94%, at least 95%, at least 96%, at least 97%, at least 98%, at least 99%, or at least 99.50% sequence identity undergo hybridization. Stringent hybridization conditions are, for example: 4×SSC at 65° C. and subsequent multiple washes in 0.1×SSC at 65° C. for approximately 1 hour. The term “stringent hybridization conditions” as used herein may also mean: hybridization at 68° C. in 0.25 M sodium phosphate, pH 7.2, 7% SDS, 1 mM EDTA and 1% BSA for 16 hours and subsequently washing twice with 2×SSC and 0.1% SDS at 68° C. Preferably, hybridization takes place under stringent conditions.

The terms “morphogenic” and “morphogenetic” are used interchangeably herein, usually in the context of a gene, wherein the gene product encoded by said gene is involved in morphogenesis, i.e., the biological process that causes an organism to develop its shape. The terms are also used in the context of any factor, including synthetic or naturally occurring transcription factors, directly or indirectly involved in the process of morphogenesis in a cell or organism. Furthermore, the terms are used in the context of the cellular pathways leading to whole plant regeneration.

The terms “nucleotide” and “nucleic acid” with reference to a sequence or a molecule are used interchangeably herein and refer to a single- or double-stranded DNA or RNA of natural or synthetic origin. The term nucleotide sequence is thus used for any DNA or RNA sequence independent of its length, so that the term comprises any nucleotide sequence comprising at least one nucleotide, but also any kind of larger oligonucleotide or polynucleotide. The term(s) thus refer to natural and/or synthetic deoxyribonucleic acids (DNA) and/or ribonucleic acid (RNA) sequences, which can optionally comprise synthetic nucleic acid analoga. A nucleic acid according to the present disclosure can optionally be codon optimized. Codon optimization implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism. Nucleic acid sequences according to the present application can carry specific codon optimization for the following non limiting list of organisms: Hordeum vulgare, Sorghum bicolor, Secale cereale, Triticale, Saccharum officinarium, Zea mays, Setaria italic, Oryza sativa, Oryza minuta, Oryza australiensis, Oryza alta, Triticum aestivum, Triticum durum, Hordeum bulbosum, Brachypodium distachyon, Hordeum marinum, Aegilops tauschii, Malus domestica, Beta vulgaris, Helianthus annuus, Daucus glochidiatus, Daucus pusillus, Daucus muricatus, Daucus carota, Eucalyptus grandis, Erythranthe guttata, Genlisea aurea, Nicotiana sylvestris, Nicotiana tabacum, Nicotiana tomentosiformis, Nicotiana benthamiana, Solanum lycopersicum, Solanum tuberosum, Coffea canephora, Vitis vinifera, Cucumis sativus, Morus notabilis, Arabidopsis thaliana, Arabidopsis lyrata, Arabidopsis arenosa, Crucihimalaya himalaica, Crucihimalaya wallichii, Cardamine flexuosa, Lepidium virginicum, Capsella bursa-pastoris, Olmarabidopsis pumila, Arabis hirsuta, Brassica napus, Brassica oleracea, Brassica rapa, Brassica juncacea, Brassica nigra, Raphanus sativus, Eruca vesicaria sativa, Citrus sinensis, Jatropha curcas, Glycine max, Gossypium ssp., or Populus trichocarpa.

As used herein, “non-native”, or “non-naturally occurring”, or “artificial”, or “synthetic” can refer to a nucleic acid or polypeptide sequence, or any other biomolecule like biotin or fluorescein that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide. A non-native sequence can refer to a 3′ hybridizing extension sequence, or a nuclear localization signal (NLS) attached to a molecule. A “synthetic transcription factor” as used herein thus refers to a molecule comprising at least two domains, a recognition domain and an activation domain not naturally occurring in nature.

An “organism” as used herein refers to an individual eukaryotic or prokaryotic life form, including inter alia an animal, plant, a fungus, or a single-celled life form. In the context of the present invention, an organism is preferably a plant or part of a plant.

The term “particle bombardment” as used herein, also named “biolistic transfection” or “biolistic bombardment” or “microparticle-mediated gene transfer”, refers to a physical delivery method for transferring a coated microparticle or nanoparticle comprising a nucleic acid or a genetic construct of interest into a target cell or tissue. The micro- or nanoparticle functions as projectile and is fired on the target structure of interest under high pressure using a suitable device, often called “gene-gun”. The transformation via particle bombardment uses a microprojectile of metal covered with the gene of interest, which is then shot onto the target cells using an equipment known as “gene-gun” (Sandford et al. 1987) at high velocity fast enough to penetrate the cell wall of a target tissue, but not harsh enough to cause cell death. For protoplasts, which have their cell wall entirely removed, the conditions are different logically. The precipitated nucleic acid or the genetic construct on the at least one microprojectile is released into the cell after bombardment and integrated into the genome or expressed transiently according to the definition given above. The acceleration of microprojectiles is accomplished by a high voltage electrical discharge or compressed gas (helium). Concerning the metal particles used it is mandatory that they are non-toxic, non-reactive, and that they have a smaller diameter than the target cell. The most commonly used are gold or tungsten. There is plenty of information publicly available from the manufacturers and providers of gene-guns and associated system concerning their general use.

The terms “plant” or “plant cell” as used herein refer to a plant organism, a plant organ, differentiated and undifferentiated plant tissues, plant cells, seeds, and derivatives and progeny thereof. Plant cells include without limitation, for example, cells from seeds, from mature and immature cells or organs, including embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. The different eukaryotic cells, for example, plant cells, can have any degree of ploidity, i.e. they may either be haploid, diploid, tetraploid, hexaploid or polyploid. Preferably a plant cell, plant or part of a plant as used herein, originates from or belongs to a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

A “promoter” refers to a DNA sequence capable of controlling expression of a coding sequence, i.e., a gene or part thereof, or of a functional RNA, i.e. a RNA which is active without being translated, for example, a miRNA, a siRNA, an inverted repeat RNA or a hairpin forming RNA. A promoter is usually located at the 5′ part of a gene. Promoter structures occur in all kingdoms of life, i.e., in bacteria, archaea, and eucaryots, where they have different architectures. The promoter sequence usually consists of proximal and distal elements in relation to the regulated sequence, the latter being often referred to as enhancers. Promoters can have a broad spectrum of activity, but they can also have tissue or developmental stage specific activity. For example, they can be active in cells of roots, seeds and meristematic cells, etc. A promoter can be active in a constitutive way, or it can be inducible. The induction can be stimulated by a variety of environmental conditions and stimuli. There exist strong promoters which can enable a high transcription of the regulated sequence, and weak promoters. Often promoters are highly regulated. A promoter of the present disclosure may include an endogenous promoter natively present in a cell, or an artificial or transgenic promoter, either from another species, or an artificial or chimeric promoter, i.e. a promoter that does not naturally occur in nature in this composition and is composed of different promoter elements. The process of transcription begins with the RNA polymerase (RNAP) binding to DNA in the promoter region, which is in the immediate vicinity of the transcription start site (TSS). A typical promoter sequence is thought to comprise some sequence motifs positioned at specific sites relative to the TSS. For example, a prokaryotic promoter is observed to have two hexameric motifs centered at or near −10 (Pribnow box) and −35 positions relative to the TSS. Furthermore, there can be an AT rich UP (“upstream”) element upstream of the −35 region. Procaryotic promoters are recognized by sigma factors as transcription factors. The structure of eukaryotic promoters is generally more complex and they have several different sequence motifs, such as TATA box, INR box, BRE, CCAAT-box and GC-box (Bucher P., J. Mol. Biol. 1990 Apr. 20; 212(4):563-78.). Eucaryotic cells posses three RNAPs, RNA polymerase I, II, and III, respectively. RNAP I generates ribosomal RNA (rRNA), RNAP II generates messenger RNA (mRNA) and small nuclear RNA (snRNA), and RNAP III generates transfer RNA (tRNA), snRNA and 5S-RNA.

The term “regulatory sequence” as used herein refers to a nucleic acid or amino acid sequence, which can direct the transcription and/or translation and/or modification of a nucleic acid sequence of interest. Regulatory sequences can comprise sequences acting in cis or acting in trans. Exemplary regulatory sequences comprise promoters, enhancers, terminators, operators, transcription factors, transcription factor binding sites, introns and the like.

The term “terminator”, as used herein, refers to DNA sequences located downstream, i.e. in 3′ direction, of a coding sequence and can include a polyadenylation signal and other sequences, i.e. further sequences encoding regulatory signals that are capable of affecting mRNA processing and/or gene expression. The polyadenylation signal is usually characterized in that it adds poly-A-nucleotides at the 3′ end of an mRNA precursor.

The terms “transient” or “transient introduction” as used herein refer to the transient introduction of at least one nucleic acid and/or amino acid sequence according to the present disclosure, preferably incorporated into a delivery vector and/or into a recombinant construct, with or without the help of a delivery vector, into a target structure, for example, a plant cell or cellular system, wherein the at least one nucleic acid or nucleotide sequence is introduced under suitable reaction conditions so that no integration of the at least one nucleic acid sequence into the endogenous nucleic acid material of a target structure, the genome as a whole, occurs, so that the at least one nucleic acid sequence will not be integrated into the endogenous DNA of the target cell. As a consequence, in the case of transient introduction, the introduced genetic construct will not be inherited to a progeny of the target structure, for example a plant cell. The at least one nucleic acid and/or amino acid sequence or the products resulting from transcription, translation, processing, post-translational modifications or complex building thereof are only present temporarily, i.e., in a transient way, in constitutive or inducible form, and thus can only be active in the target cell for exerting their effect for a limited time. Therefore, the at least one sequence introduced via transient introduction will not be heritable to the progeny of a cell. The effect mediated by at least one sequence or effector introduced in a transient way can, however, potentially be inherited to the progeny of the target cell. A “stable” introduction therefore implies the integration of a nucleic acid or nucleotide sequence into the genome of a target cell or cellular system of interest, wherein the genome comprises the nuclear genome as well as the genome comprised by further organelles.

The term “variant(s)” as used herein in the context of amino acid or nucleic acid sequences is intended to mean substantially similar sequences. For nucleic acid sequences, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For nucleic acid sequences, conservative variants include those sequences that, because of the degeneracy of the genetic code, encode the same amino acid sequence as a reference sequence of the present disclosure. A variant of a given nucleic acid sequence will thus also include synthetically derived nucleic acid sequences, such as those generated, for example, by using site-directed mutagenesis but which still encode the same protein as the reference sequence. Generally, variants of a particular polynucleotide of the disclosure will have at least about 60%, 65%, 70%, 75%, 80%, 85%, 90%, 91%, 92%, 93%, 94%, 95%, 96%, 97%, 98%, 99% or more sequence identity to that particular nucleic acid sequence as determined by sequence alignment programs and parameters described further below under this section.

A “variant” amino acid sequence, polypeptide or protein (said terms being used interchangeably herein) means an amino acid sequence derived from the native amino acid sequence by deletion or addition of one or more amino acids at one or more internal sites in the native protein and/or substitution of one or more amino acids at one or more sites in the native protein. Variant amino acid sequences according to the present disclosure are biologically active, that is they continue to possess the desired biological activity of the native protein. Active variants of a native amino acid sequence of the disclosure will have at least about 65%, 70%, 75%, 80%, 85%, 90%, 91%, 92%, 93%, 94%, 95%, 96%, 97%, 98%, 99% or more sequence identity to the amino acid sequence for the native amino acid sequence as determined by sequence alignment programs and parameters described further below under this section.

Whenever the present disclosure relates to the percentage of identity of nucleic acid or amino acid sequences to each other these values define those values as obtained by using the EMBOSS Water Pairwise Sequence Alignments (nucleotide) programme (www.ebi.ac.uk/Tools/psa/emboss_water/nucleotide.html) nucleic acids or the EMBOSS Water Pairwise Sequence Alignments (protein) programme (www.ebi.ac.uk/Tools/psa/emboss_water/) for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified Smith-Waterman algorithm (see www.ebi.ac.uk/Tools/psa/and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5. The skilled person is well aware of the fact that, for example, a sequence encoding a protein can be “codon-optimized” if the respective sequence is to be used in another organism in comparison to the original organism a molecule originates from.

### DETAILED DESCRIPTION

The person skilled in the art will understand that the herein described aspects and embodiments should not be construed to be confined to the specific context in which they are disclosed, but rather that the aspects and embodiments described throughout the present specification can be combined with each other independently from their specific context.

The present invention is based on the finding that the selective modulation of the gene expression of endogenous genes by using specifically defined synthetic transcription factors (STFs) provides a suitable tool for specific temporal and spatial regulation of a gene of interest. In turn, this provides the basis for the optimization of transformation and genome editing approaches and thus provides higher frequencies in transformation/editing which in turn allows improved methods in agricultural biotechnology.

For example, instead of using the nucleotide sequences encoding the morphogenic genes, for example, BBM and WUS, as isolated or heterologous expression cassettes, it is possible to use specifically designed synthetic transcriptional modulators, such as TAL effectors or disarmed CRISPR/nuclease systems and others, to induce expression of the endogenous morphogenic genes to reprogram the cell and to induce cell division and regeneration at a specific time point in a transient way without the need to introduce a transgenic morphogenic effector, or the sequence encoding the same, into a cell or plant of interest. These principle findings were expanded to establish synthetic transcription factors (STFs) comprising at least one activation or silencing domain to specifically up- or downregulate the expression of a target gene in an inducible way. In turn, the direct effect of said specifically designed artificial STFs was then used in a variety of methods of molecular biology to synergistically profit from the modulation effect for optimizing transformation, gene editing, or targeted silencing, wherein these methods can be employed for plant breeding and for potential therapeutic applications. In one aspect of the present invention, approaches were established to generate plants by using the synthetic transcription factors specific for BBM and WUS to induce cell division and regeneration of plant cells, which findings were then extrapolated to further methods and uses based on a variety of synthetic transcription factors. In turn, these specific transcription factors allow the provision of methods of improving the efficiency of plant transformation and/or regeneration of transgenic plants by using synthetic transcription factors specific for endogenous morphogenic genes which can reprogram the cell and induce cell division in a large variety of plant species, including those species or varieties known to be hard to transform and regenerate to dramatically increase the transformation efficiency of a variety of species and further of a variety of different cell types including those cell types being recalcitrant to transformation in standard settings. The present invention thus relates to both the molecular tools specific for a morphogenic gene of interest which is targeted for modulation, preferably activation, i.e., the present invention relates to the specific synthetic transcription factors and the sequences encoding the same, as well as to methods of using these specific synthetic or artificial transcription factors in a targeted way to optimize transformation and transfection based methods of plant biotechnology, in particular genome editing based methods, or methods for optimizing the transformation rates of transformation recalcitrant plant cells.

For the first time it was demonstrated in the context of the present invention, that Cpf1-based transcription activation systems can be successfully employed in plants to modulate the expression of endogenous target genes. Advantageously, the provided means and methods allow to target enogenous genes having AT-rich promoter regions, which was previously not possible. The system is easy to use for targeting multiple genomic regions simultaneously by providing specifically designed guide RNA arrays and allows to transiently modulate expression without introducing transgenes.

In one aspect, there is disclosed a synthetic transcription factor (STF), or a nucleotide sequence encoding the same, which may comprise at least one recognition domain and at least one gene expression modulation domain, in particular at least one activation domain, wherein the synthetic transcription factor may be configured to modulate the expression of a morphogenic gene in a cellular system.

A “modulation” of the expression of any endogenous gene, preferably a morphogenic gene, as disclosed herein includes both gene activation and gene repression as defined above. Such a modulation can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP (see, e.g., Mistili & Spector, (1997) Nature Biotechnology 15: 961-964). For morphogenic genes, a modulation of gene expression can also be monitored by visual means, including microscopy, observation of plant development and the like to monitor changes in any functional effect of gene expression. According to the various aspects of the present invention, a synthetic transcription factor as disclosed herein will preferably act on the transcriptional level and will thus modulate the transcription of at least one gene of interest, preferably a morphogenic gene of interest. In certain embodiments, the at least one synthetic transcription factor may be specifically designed to upregulate the transcription of a gene of interest, preferably a morphogenic gene of interest.

A “cellular system” as used herein refers to at least one element comprising all or part of the genome of a cell of interest to be modified. The cellular system may thus be any in vivo or in vitro system, including also a cell-free system. The cellular system thus comprises and provides the target genome or genomic sequence to be modified in a suitable way, i.e., in a form accessible to a genetic modification or manipulation. The cellular system may thus be selected from, for example, a eukaryotic cell, including a plant cell, or the cellular system may comprise a genetic construct as defined above comprising all or parts of the genome of a eukaryotic cell to be modified in a highly targeted way. The cellular system may be provided as isolated cell or vector, or the cellular system may be comprised by a network of cells in a tissue, organ, material or whole organism, either in vivo or as isolated system in vitro. In this context, the “genetic material” of a cellular system can thus be understood as all, or part of the genome of an organism the genetic material of which organism as a whole or in part is present in the cellular system to be modified.

In one aspect, the present invention provides a cellular system which may be obtained by a method according to any one of the above aspects and embodiments.

In one embodiment according to the various aspects of the present invention, the synthetic transcription factor may be designed to modulate the transcription of a morphogenic gene, wherein the morphogenic gene may be selected from the group consisting of BBM, WUS (Zuo et al., 2002, Plant J., 30(3):349-359), including WUS2 (Nardmann and Werr, 2006, Mol. Biol. Evol., 23:22492-22502), a WOX gene, a WUS or BBM homologue, Lec1, Lec2, WIND1, ESR1, PLT3, PLT5, or PLT7, IPT, IPT2, Knotted1, and RKD4.

According to the various aspects and embodiments of the present invention, the morphogenic gene may be selected from sequences having coding sequences of NM_001112491.1 (SEQ ID NO: 199), NM_127349.4 (SEQ ID NO: 200), NC_025817.2, KT285832.1 (SEQ ID NO: 201), KT285833.1 (SEQ ID NO: 202), KT285834.1 (SEQ ID NO: 203), KT285835.1 (SEQ ID NO: 204), KT285836.1 (SEQ ID NO: 205), KT285837.1 (SEQ ID NO: 206), XM_008676474.2 (SEQ ID NO: 207), CM007649.1, NM_103997.4 (SEQ ID NO: 208), XM_010675298.2 (SEQ ID NO: 209), XM_010675704.2 (SEQ ID NO: 210), AB458519.1 (SEQ ID NO: 211), AB458518.1 (SEQ ID NO: 212), AK451358.1 (SEQ ID NO: 213), AK335319.1 (SEQ ID NO: 214), KU593504.1 (SEQ ID NO: 215) or KU593503.1 (SEQ ID NO: 216).

In a further embodiment, there is provided a synthetic transcription factor, wherein the morphogenic gene comprises a nucleotide sequence selected from the group consisting of (i) a nucleotide sequence set forth in any one of SEQ ID NOs: 199 to 237, (ii) a nucleotide sequence having the coding sequences of the nucleotide sequence set forth in any one of SEQ ID NOs: 199 to 237, (iii) a nucleotide sequence complementary to the nucleotide sequence of (i) or (ii), (iv) a nucleotide sequence having at least 50%, 60%, 70%, 75%, 80%, 85%, 90%, 91%, 92%, 93%, 94%, 95%, 96%, 97%, 98%, or 99% identity, preferably over the whole length, to the the nucleotide sequence of (i), (ii) or (iii), (v) a nucleotide sequence hybridzing the nucleotide sequence of (iii) under stringent conditions, (vi) a nucleotide sequence encoding a protein comprising the amino acid sequence set forth in any one of SEQ ID NOs: 238 to 258, (vii) a nucleotide sequence encoding a protein comprising the amino acid sequence at least 50%, 60%, 70%, 75%, 80%, 85%, 90%, 91%, 92%, 93%, 94%, 95%, 96%, 97%, 98%, or 99% identical to a sequence set forth in any one of SEQ ID NOs: 238 to 258, or (viii) a nucleotide sequence encoding a homologue, analogue or orthologue of protein comprising the amino acid sequence set forth in any one of SEQ ID NOs: 238 to 258.

In particular, the Wuschel (WUS) polypeptide has been identified as key player in the initiation and maintenance of the apical meristem, which contains a pool of pluripotent stem cells (Endrizzi et al., 1996, Plant Journal 10:967-979). Arabidopsis plants mutant for the WUS gene contain stem cells that are misspecified and that appear to undergo differentiation. WUS encodes a homeodomain protein, which functions as a transcriptional regulator (Mayer et al., 1998, Cell 95:805-815, US 2004/166563 A1). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level. WUS expression can be sufficient to induce meristem cell identity and the expression of the stem cell marker CLV3 (Brand et al. (2000) Science 289:617-619; Schoof et al. (2000) Cell 100:635-644). Constitutive expression of WUS in Arabidopsis has been shown to lead to adventitious shoot proliferation from leaves (in planta) (US 2004/166563 A1).

Further WUS/WOX homeobox polypeptides and genes encoding the same are known to the skilled person and can be targeted by the synthetic transcription factors and/or using the methods as disclosed herein. A WUS homeobox polypeptide may be selected from WUS 1, WUS2, WUS 3, WOX2A, WOX4, WOX5, or WOX9 polypeptide (van der Graaff et al., 2009, Genome Biology 10:248), or homolouges thereof. The WUS homeobox polypeptide can be a monocot WUSAVOX homeobox polypeptide. In various aspects, WUS homeobox polypeptide can be a barley, maize, millet, oats, rice, rye, Setaria sp., sorghum, sugarcane, switchgrass, triticale, turfgrass, or wheat WUSAVOX homeobox polypeptide. Alternatively, the WUS homeobox polypeptide can be a dicot WUS homeobox polypeptide (see WO 2017/074547 A1). In addition, the AP2/ERF family of proteins is a plant-specific class of putative transcription factors that have been shown to regulate a wide-variety of developmental processes and are characterized by the presence of a AP2/ERF DNA binding domain. The AP2/ERF proteins have been subdivided into two distinct subfamilies based on whether they contain one (ERF subfamily) or two (AP2 subfamily) DNA binding domains. One member of the AP2 family that has been implicated in a variety of critical plant cellular functions is the Baby Boom (BBM) protein. The BBM protein from Arabidopsis is preferentially expressed in seed and has been shown to play a central role in regulating embryo-specific pathways. Overexpression of BBM has been shown to induce spontaneous formation of somatic embryos and cotyledon-like structures on seedlings. See, Boutiler et al. (2002) The Plant Cell 14:1737-1749. Thus, members of the AP2 (APETALA2) protein family promote cell proliferation and morphogenesis during embryogenesis. Such activity finds potential use in promoting apomixis in plants.

Another morphogenic target according to the present invention is Ovule Development Protein 2 (ODP2). It is also a member of the AP2 family of proteins. ODP2 polypeptides of the invention contain two predicted APETALA2 (AP2) domains and are members of the AP2 protein family (PFAM Accession PF00847). The AP2 domains of the maize ODP2 polypeptide are located from about amino acids S273 to N343 and from about S375 to R437 of SEQ ID NO:2). The AP2 family of putative transcription factors have been shown to regulate a wide range of developmental processes, and the family members are characterized by the presence of an AP2 DNA binding domain. This conserved core is predicted to form an amphipathic alpha helix that binds DNA. The AP2 domain was first identified in APETALA2, an Arabidopsis protein that regulates meristem identity, floral organ specification, seed coat development, and floral homeotic gene expression. The AP2 domain has now been found in a variety of proteins.

Therefore, morphogenic effectors of the AP2 family play critical roles in a variety of important biological events including development, plant regeneration, cell division, etc, these morphogenic effectors are valuable for the field of agronomic development to identify and characterize novel AP2 family members and develop novel methods to modulate embryogenesis, transformation efficiencies, and yield related traits, including oil content, starch content and the like in a plant, and are relevant targets of the synthetic transcription factors and the associated methods of the present invention.

Many attempts have been made to utilize the modulation of WUS, BBM and other morphogenic genes to improve transformation efficiency, to stimulate plant cell growth, including stem cells, to stimulate organogenesis, to stimulate somatic embryogenesis, to induce apomixis, and to provide a positive selection for cells and the like. The ability to stimulate organogenesis and/or somatic embryogenesis may be used to generate an apomictic plant. Apomixis has economic potential because it can cause any genotype, regardless of how heterozygous, to breed true. It is a reproductive process that bypasses female meiosis and syngamy to produce embryos genetically identical to the maternal parent. With apomictic reproduction, progeny of adaptive or hybrid genotypes would maintain their genetic fidelity throughout repeated life cycles. In addition to fixing hybrid vigor, apomixis can make possible commercial hybrid production in crops where efficient male sterility or fertility restoration systems for producing hybrids are not available. Apomixis can make hybrid development more efficient. It also simplifies hybrid production and increases genetic diversity in plant species with good male sterility.

Still, all current approaches of modulating the endogenous morphogenic gene pool of plant cells presently rely on the provision of genes encoding the morphogenic gene of interest to overexpress the respective morphogenic gene. Therefore, current methods rely on the stable or transient introduction and/or overexpression of a morphogenic gene of interest. In contrast, the present invention identified a solution to specifically design a synthetic transcription factor to modulate the transcription level of a morphogenic gene of interest, preferably in a transient and/or regulatable way, without the need to introduce an exogenous transgenic sequence of a morphogenic gene product, or the sequence encoding the same. This paves the way to provide methods for increasing the transformation efficiency in plants, e.g., for complex genome editing methods, even in transformation recalcitrant plants, and to provide methods for providing haploid or double haploid organisms or cellular systems.

A variety of different molecules can be used as the at least one recognition domain according to the present invention. According to the various aspects and embodiments disclosed herein, a recognition domain represents a protein domain, optionally as a fusion molecule, which possesses site-specific DNA recognition and thus binding and/or interaction activity. A recognition domain can be a domain from a naturally occurring protein, or the recognition domain may be a fragment of such a protein. Preferably, the at least one recognition domain has been specifically engineered to optimize the target specificity thereof for binding to a region of a morphogenic gene of interest, or to a region surrounding a morphogenic gene of interest.

More than one recognition domains may be used according to the present invention to increase the target specificity and/or binding characteristics to optimize modulation of the at least one morphogenic gene of interest.

In one embodiment, the synthetic transcription factor may comprise at least one recognition domain, or a fragment, of a molecule selected from the group consisting of at least one TAL effector, at least one disarmed CRISPR/nuclease system, at least one Zinc-finger domain, and at least one disarmed homing endonuclease, or any combination thereof.

In a further embodiment, the synthetic transcription factor may comprise at least one disarmed CRISPR/nuclease system selected from a CRISPR/dCas9 system, a CRISPR/dCpf1 system, a CRISPR/dCasX system or a CRISPR/dCasY system, or any combination thereof, wherein the at least one disarmed CRISPR/nuclease system, if present, comprises at least one guide RNA.

Naturally occurring DNA-binding transcription factors generally contain a minimum of two domains: a DNA-binding domain (DBD) and a transcriptional activation domain (TAD) (Latchman, 2008; Ptashne and Gann, 2002).

TAL effectors of plant pathogenic bacteria in the genus Xanthomonas play important roles in disease, or trigger defense, by binding host DNA and activating effector-specific host genes (see, e.g., Gu et al. (2005) Nature 435:1122; Römer et al. (2007) Science 318:645). Specificity depends on an effector-variable number of imperfect, typically 34 amino acid repeats (Schornack et al. (2006) J. Plant Physiol. 163:256). Polymorphisms are primarily at repeat positions 12 and 13, which are referred to herein as the repeat variable-diresidue (RVD). RVDs of TAL effectors correspond to the nucleotides in their target sites in a direct, linear fashion, one RVD to one nucleotide, with some degeneracy and no apparent context dependence. This finding represents a valuable mechanism for protein-DNA recognition that enables target site prediction for new target specific TAL effector. Therefore, TAL effectors are not only useful in research and biotechnology as targeted chimeric nucleases that can facilitate homologous recombination for GE approaches. TAL effectors per se do not comprise a nuclease domain. The so-called transcription activator-like effector endonucleases (TALENs) represent artificial or synthetic molecules combining the TAL effector function with a nuclease function for allowing the insertion of a site-specific DNA cleavage. For example, the TAL effector may enter the host cell nucleus via a C-terminal nuclear localization domain and may specifically activate the corresponding host gene through binding to an effector binding element in the promoter region of the host gene. The central domain of highly conserved, 33-35-amino acid repeats, each containing hypervariable dinucleotides or RVDs at positions 12 and 13, are responsible for the recognition of specific host gene promoter sequences. Each TAL effector wraps around the DNA in a right-handed superhelix positioning the second residue of each RVD into the major groove, where it contacts an individual nucleotide in the forward strand. These interactions define the specificity of each TAL effector. A C-terminal acidic activation domain then activates or enhances the expression of the corresponding endogenous gene, presumably by directly engaging the host RNA polymerase complex.

The modular mechanism by which TAL effectors recognize specific DNA sequences allows for the identification and design of artificial repeat arrays in the recognition domain of a TAL effector thereby designing TAL effectors which are capable to specifically induce expression of an endogenous gene of interest.

Computational analysis of genomic target sites of natural TALEs showed a preferential occurrence in apparent core promoter regions of −300 to +200 bp around the transcriptional start site (TSS) (Grau et al., PLoS Comput Biol. 2013; 9). Previous studies based on the TALEs AvrBs3, AvrXa7, and AvrXa27 showed that they shift the natural TSS of target genes around 40-60 bp downstream of the position at which the TALE is binding the DNA. Moving the AvrBs3-box in the Bs3 promoter to a position further upstream resulted in a concomitant upstream shift of the TSS. These observations led to the impression that TALEs control the onset and the place of transcription functionally analogous to the TATA-binding protein (Kay et al., Science. 2007; 318: 648-651).

Therefore, TAL effector binding domains represent suitable recognition domains according to the various aspects and embodiments of the present invention, as the binding and recognition specificities can be fine-tuned for a target site of interest. Therefore, expression, preferably transcription, of a morphogenic gene of interest can be modulated in a highly targeted manner, as at least one custom TAL effector can be designed as the at least one recognition domain of a synthetic transcription factor.

Functioning as heterologous transcription factors in their natural environment, TAL effectors (Yang et al., 2006) are delivered via the bacterial type Ill secretion system into host cells (Szurek et al., 2002), where C-terminal nuclear localization signals direct them to the nucleus (Gurlebeck et al., 2005; Szurek et al., 2001, 2002; Van den Ackerveken et al., 1996; Yang and Gabriel, 1995). The central domain of highly conserved, 33-35-amino-acid repeats, each containing hypervariable residues at positions 12 and 13 (the RVD), directs the recognition of specific host gene promoter sequences called effector binding elements (EBEs) (Boch et al., 2009; Moscou and Bogdanove, 2009). Each TAL effector wraps the DNA in a right-handed superhelix, positioning the second residue of each RVD into the major groove, where it contacts an individual nucleotide in the forward strand (Deng et al., 2012; Mak et al., 2012). Collectively, these interactions define, in a predictable way, the number and identity of adjacent nucleotides that constitute the EBE. A C-terminal acidic activation domain (AD) then activates or enhances transcription, presumably by directly engaging the host RNA polymerase complex (cf. Hummel et al., Molecular Plant Pathology, 2017, 18(1), 55-66).

In contrast to the teaching of the prior art, the present invention is partly based on the finding that synthetic TAL effector-based transcription factors, disarmed ZFP-based transcription factors, or disarmed CRISPR-based transcription factors specific for endogenous nucleotide sequences located at a specific upstream or downstream position relative to the start codon of a gene of interest, preferably a morphogenic gene, for example, BBM and WUS, can induce transcription and expression of said genes in a plant cell thereby boosting the regeneration frequency of such plant. Notably, this efficiency can be enhanced in case non-classical regulation regions outside of a TATA-box or the promoter region are targeted, whereas naturally occurring transcription factors as well as commercially available transcription factors usually exert their function by binding to a region within the promoter region of a gene of interest. There is evidence that the transcriptional activation is higher in proximity to the TATA box compared to directly targeting the TATA region. The transcription factors of the present invention based on the various different TAL effector, CRISPR, zinc-finger or homing endonuclease based recognition domain thus comprise a different architecture allowing a better and more precise modulation and regulation of a morphogenic gene of interest.

Therefore, it can be an advantage of the synthetic transcription factors and the methods of the present invention that the synthetic transcription factors can also act on TATA-less genes, or outside a TATA region, if correctly designed to comprise optimum recognition and activation regions. In certain embodiments, at least one recognition domain may also target a TATA region of a gene of interest.

For example, a TAL effector DNA binding domain can be specific for a target DNA, wherein the DNA binding domain comprises a plurality of DNA binding repeats, each repeat comprising a RVD that determines recognition of a base pair in the target DNA, wherein each DNA binding repeat is responsible for recognizing one base pair in the target DNA, and wherein the TALEN comprises one or more of the following RVDs: HD for recognizing C; NG for recognizing T; NI for recognizing A; NN for recognizing G or A; NS for recognizing A or C or G or T; N* for recognizing C or T; HG for recognizing T; H* for recognizing T; IG for recognizing T; NK for recognizing G; HA for recognizing C; ND for recognizing C; HI for recognizing C; HN for recognizing G; NA for recognizing G; SN for recognizing G or A; and YG for recognizing T. The TALEN can comprise one or more of the following RVDs: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T; HG for recognizing T; H* for recognizing T; and IG for recognizing T.

Zinc finger proteins (ZFPs) are proteins that can bind to DNA in a sequence specific manner. Zinc fingers were first identified in the transcription factor TFIIIA from the oocytes of the African clawed toad, Xenopus laevis. An exemplary motif characterizing one class of these proteins (Cys2His2 class) is Xaa-Cys-Xaa-Cys-Xaa-His-Xaa-His (SEQ ID NO: 313), where Xaa is any amino acid. Individual fingers from these proteins have a simple ββα structure that folds around a central zinc ion, and tandem sets of fingers can contact neighboring subsites of 3-4 base pairs along the major groove of the DNA (Pabo et al. (2001) “Design and selection of novel Cys2His2 zinc finger proteins”. Ann. Rev. Biochem. 70: 313-40). A single zinc finger domain is about 30 amino acids in length, and several structural studies have demonstrated that it contains a beta turn (containing the two invariant cysteine residues) and an alpha helix (containing the two invariant histidine residues), which are held in a particular conformation through coordination of a zinc atom by the two cystines and the two histidines. Several other class of zinc finger proteins are known, e.g., the treble-clef class comprising a motif consisting of a β-hairpin at the N-terminus and an α-helix at the C-terminus that each contribute two ligands for zinc binding, although a loop and a second β-hairpin of varying length and conformation can be present between the N-terminal β-hairpin and the C-terminal α-helix, or zinc ribbon like ZFPs having a fold being characterized by two beta-hairpins forming two structurally similar zinc-binding sub-sites.

For genome editing (GE) purposes techniques of molecular biology can be used to alter the DNA-binding specificity of zinc fingers and tandem repeats of such engineered zinc fingers can be used to target desired genomic DNA sequences (Jamieson et al., “Drug discovery with engineered zinc-finger proteins”. Nature Reviews. Drug Discovery. 2 (5): 361-8.). Fusing a second protein domain such as a transcriptional activator or repressor to an array of engineered zinc fingers that bind near the promoter of a given gene can be used to alter the transcription of that gene. Fusions between engineered zinc finger arrays and protein domains that cleave or otherwise modify DNA can also be used to target those activities to desired genomic loci. The most common applications for engineered zinc finger arrays include zinc finger transcription factors and zinc finger nucleases. Typical engineered zinc finger arrays have between 3 and 6 individual zinc finger motifs and bind target sites ranging from 9 basepairs (bp) to 18 bp in length.

Meganucleases are endodeoxyribonucleases characterized by a large recognition site (double-stranded DNA sequences of 12 to 40 base pairs). As a result, this site generally occurs only once in any given genome. Meganucleases can be used to achieve very high levels of gene targeting efficiencies in mammalian cells and plants (Rouet et al., Mol. Cell. Biol., 1994, 14, 8096-106; Choulika et al., Mol. Cell. Biol., 1995, 15, 1968-73). Among meganucleases, the LAGLIDADG family of homing endonucleases has become a valuable tool for the study of genomes and genome engineering over the past years.

Disarmed, i.e., nuclease-deficient, homing endonucleases (HEs) represent a suitable class of recognition domains according to the present invention. HEs are a widespread family of natural meganucleases including hundreds of proteins (Chevalier and Stoddard, Nucleic Acids Res., 2001, 29, 3757-74). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus (Kostriken et al., Cell; 1983, 35, 167-74; Jacquier and Dujon, Cell, 1985, 41, 383-94). Given their natural function and their exceptional cleavage properties in terms of efficacy and specificity, HEs provide ideal scaffolds to derive novel endonucleases for genome engineering. One family of HEs is called the LAGLIDADG family. LAGLIDADG (SEQ ID NO: 314) refers to the only sequence actually conserved throughout the family and is found in one or (more often) two copies in the protein. Proteins with a single motif, such as I-CreI, form homodimers and cleave palindromic or pseudo-palindromic DNA sequences, whereas the larger, double motif proteins, such as I-SceI are monomers and cleave non-palindromic targets. Seven different LAGLIDADG proteins have been crystallized, and they exhibit a very striking conservation of the core structure, that contrasts with the lack of similarity at the primary sequence level (Jurica et al., Mol. Cell., 1998, 2, 469-76; Chevalier et al., Nat. Struct. Biol., 2001, 8, 312-6; Chevalier et al. J. Mol. Biol., 2003, 329, 253-69). Analysis of I-Cre structure bound to its natural target shows that in each monomer, eight residues (Y33, Q38, N30, K28, Q26, Q44, R68 and R70) establish direct interactions with seven bases at positions ±3, 4, 5, 6, 7, 9 and 10 (Jurica et al., 1998). In addition, some residues establish water-mediated contact with several bases; for example, S40 and N30 with the base pair at position 8 and −8 (Chevalier et al., 2003). The catalytic core is central, with a contribution of both symmetric monomers/domains. HEs having a modified cleavage site are known to the skilled person and can be used to define a disarmed HE as the at least one recognition domain according to the present invention.

According to the various aspects and embodiments according to the present invention, zinc finger proteins and domains derived therefrom can be used as the at least one recognition domain, which at least one recognition domain can be designed to fulfill the recognition properties of a synthetic transcription factor according to the present invention.

Besides TAL effectors, disarmed ZFPs and meganucleases, non-functional CRISPR/nuclease systems can be used to specifically target morphogenic genes and to boost regeneration of plant cells. In these systems, a CRISPR nuclease such as Cas9, Cfp1, CasX and/or CasY is used in which the nuclease activity has been turned off to avoid cleavage of the target genomic sequences. The target specificity of the non-functional CRISPR/nuclease system is determined by crRNAs and/or sgRNAs specific for the upstream nucleotide promoter region of an endogenous morphogenic gene of interest. An activation domain which is fused to the CRISPR/nuclease system then recruits the transcription machinery to the gene locus thereby inducing the expression of the endogenous morphogenic gene of interest. Notably, the use of at least one guide RNA can dramatically increase the target specificity, as this CRISPR nucleic acid sequence additionally contributes in the recognition of genomic target DNA of interest. Moreover, the dual recognition properties of a disarmed CRISPR nuclease and the guide RNA allows a higher degree of flexibility in designing synthetic transcription factor recognition domains according to the present invention which in turn provides a better recognition and thus modulation activity of a morphogenic gene of interest.

In a preferred embodiment of the various aspects of the present invention, the at least one recognition domain is, or is a fragment of at least one disarmed CRISPR/nuclease system.

A CRISPR system in its natural environment describes a molecular complex comprising at least one small and individual non-coding RNA in combination with a Cas nuclease or another CRISPR nuclease like a Cpf1 nuclease (Zetsche et al., 2015, supra) which can produce a specific DNA double-stranded break. Presently, CRISPR systems are categorized into 2 classes comprising five types of CRISPR systems, the type II system, for instance, using Cas9 as effector and the type V system using Cpf1 as effector molecule (Makarova et al., Nature Rev. Microbiol., 2015). In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or gRNA combining the function of a crRNA and/or a tracrRNA (Makarova et al., 2015, supra). The immune response mediated by CRISPR/Cas in natural systems requires CRISPR-RNA (crRNA), wherein the maturation of this guiding RNA, which controls the specific activation of the CRISPR nuclease, varies significantly between the various CRISPR systems which have been characterized so far. Firstly, the invading DNA, also known as a spacer, is integrated between two adjacent repeat regions at the proximal end of the CRISPR locus. Type II CRISPR systems, for example, can code for a Cas9 nuclease as key enzyme for the interference step, which system contains both a crRNA and also a trans-activating RNA (tracrRNA) as the guide motif. These hybridize and form double-stranded (ds) RNA regions which are recognized by RNAsellI and can be cleaved in order to form mature crRNAs. These then in turn associate with the Cas molecule in order to direct the nuclease specifically to the target nucleic acid region. Recombinant gRNA molecules can comprise both the variable DNA recognition region and also the Cas interaction region and thus can be specifically designed, independently of the specific target nucleic acid and the desired Cas nuclease. As a further safety mechanism, PAMs (protospacer adjacent motifs) must be present in the target nucleic acid region; these are DNA sequences which follow on directly from the Cas9/RNA complex-recognized DNA. The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337: 816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973 A1). For Cpf1 nucleases it has been described that the Cpf1-crRNA complex, without a tracrRNA, efficiently recognize and cleave target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

Presently, for example, Type II systems relying on Cas9, or a variant or any chimeric form thereof, as endonuclease have been modified for genome engineering. Synthetic CRISPR systems consisting of two components, a “guide RNA” (gRNA) also called “single guide RNA” (sgRNA) or “CRISPR nucleic acid sequence” herein and a non-specific CRISPR-associated endonuclease can be used to generate knock-out cells or animals by co-expressing a gRNA specific to the gene to be targeted and capable of association with the endonuclease Cas9. Notably, the gRNA is an artificial molecule comprising one domain interacting with the Cas or any other CRISPR effector protein or a variant or catalytically active fragment thereof and another domain interacting with the target nucleic acid of interest and thus representing a synthetic fusion of crRNA and tracrRNA (as “single guide RNA” (sgRNA) or simply “gRNA”). The genomic target can be any ˜20 nucleotide DNA sequence, provided that the target is present immediately upstream of a PAM sequence. The PAM sequence is of outstanding importance for target binding and the exact sequence is dependent upon the species of Cas9 and, for example, reads 5′ NGG 3′ or 5′ NAG 3′ (Standard IUPAC nucleotide code) (Jinek et al., Science 2012, supra) fora Streptococcus pyogenes derived Cas9. The PAM sequence for Cas9 from Staphylococcus aureus is NNGRRT or NNGRR(N). Many further variant CRISPR/Cas9 systems are known, including inter alia, Neisseria meningitidis Cas9 cleaving the PAM sequence NNNNGATT. A Streptococcus thermophilus Cas9 cleaving the PAM sequence NNAGAAW. Using modified Cas nucleases, targeted single-strand breaks can be introduced into a target sequence of interest. The combined use of such a Cas nickase with different recombinant gRNAs highly site-specific DNA double-strand breaks can be introduced using a double nicking system. Using one or more gRNAs can further increase the overall specificity and reduce off-target effects.

A third variant of a Cas or Cpf1 nuclease of particular interest for the purpose of the present invention is a nuclease-deficient Cas9 (dCas9) or dCpf1 (Qui et al, 2013, Cell, 154, 442-451). Mutations H840A in the HNH domain and D10A in the RuvC domain of Cas9 inactivate cleavage activity, but do not prevent DNA binding (Gasiunas et al., 2012, Proc. Natl. Acad. Sci. U.S.A., 111, E2579-2586). Therefore, these variants, if properly configured can be repurposed to sequence-specifically target a region of the genome without cleavage.

Cpf1 may be derived e.g. from Acidaminococcus sp. BV3L6 (AsCpf1) or from Lachnospiracea bacterium ND2006 (LbCpf1) as described in Tang et al. (Tang et al. (2017), A CRISPR/Cpf1 system for efficient genome editing and transcriptional repression in plants. Nature Plants, 3:17018). Preferred dLbCpf1 variants are represented by SEQ ID NOs: 282-284 and 288-290.

A CRISPR/Cpf1 system allows to target AT-rich promoter regions and can be used in a wide variety of crop plants. Because of the RNAse activity of Cpf1 being able to process multiple crRNAs from a single transcript, a Cpf1-based transcription regulation system has the advantage over commonly known Cas9-based systems that it can be easily applied for multiplexed gene regulation.

In a preferred embodiment of the various aspects of the present invention the at least one disarmed CRISPR/nuclease system is therefore a CRISPR/dCpf1 system, wherein the at least one disarmed CRISPR/nuclease system comprises at least one guide RNA.

The Cpf1-based transcription regulation system is highly specific and flexible and allows the simultaneous activation/suppression of multiple genes by the use of a guide RNA array targeting multiple genomic regions. Furthermore, the Cpf1-based system achieves elevated gene expression without the need of introducing exogenous polynucleotide or polypeptide sequences of the gene of interest. It is therefore possible to transiently induce gene expression of endogenous genes in transgene-free environment. Furthermore, the Cpf1-based system provides means to target AT-rich sequences which was not possible with the so far known Cas9-based transcription regulation systems which show a strong preference towards GC-rich regions. The system thus provides a powerful tool for transcriptional activation and/or suppression of endogenous target genes of interest in a plant cell. It is easy to use and suitable for simultaneously targeting multiple genes. Importantly, it is for the first time shown that Cpf1-based transcriptional activation works in plant cells. Although the prior art describes Cpf1-based gene suppression in A. thaliana, Cpf1-based transcriptional activation has not been shown in plants so far, suggesting that replacement of a transcription suppression domain by a transcription activation domain is not straightforward and requires elaborate configuration and testing of the right linker and activation domain sequences.

In one embodiment according to the various aspects of the present invention, the recognition domain may comprise at least one gRNA of a CRISPR complex. In certain embodiments, more than one gRNA may be present, e.g. an array of gRNAs may be used. The expression of multiple guide RNAs in a single cell or cellular system, e.g., the expression of two, three, four, five, or more gRNAs, may enable a synergistic modulation of endogenous gene targets, thereby enabling combinatorial control of endogenous gene expression over a wide dynamic range due to the fact that the at least one gRNA as recognition moiety if a STF according to the present invention can provide additional target specificity to the STF and reduce off-target effects, particularly when the STFs are designed to target a gene in a huge eukaryotic genome. Each gRNA may target an independent regulation/recognition region.

In one embodiment according to the various aspects of the present invention, the synthetic transcription factor may be configured to modulate expression, preferably transcription, of the morphogenic gene by binding to a regulation region located at a certain distance in relation to the start codon.

The “regulation region” as used herein refers to the binding site of at least one recognition domain to a target sequence in the genome at or near a morphogenic gene of interest. There may be two discrete regulation regions, or there may be overlapping regulation regions, depending on the nature of the at least one activation domain and the at least one recognition domain as further disclosed herein, which different domains of the synthetic transcription factor of the present invention can be assembled in a modular manner.

In certain embodiments, the at least one recognition domain may target at least one sequence (recognition site) relative to the start codon of a gene of interest, which sequence may be at least 1.000 bp upstream (−) or downstream (+), −700 bp to +700 bp, −550 bp to +500 bp, or −550 bp to +425 bp relative to of the start codon of a gene of interest. Promoter-near recognizing recognition domains might be preferable in certain embodiments, whereas it represents an advantage of the specific STFs of the present invention that the targeting range of the STFs is highly expanded over conventional or naturally occurring TFs. As the recognition and/or the activation domains can be specifically designed and constructed to specifically identify and target hot-spots of modulation.

In certain embodiments, the at least one recognition site may be −169 bp to −4 bp, −101 bp to −48 bp, −104 to −42 bp, or −175 to +450 bp (upstream (−) or downstream (+), respectively) relative to the start codon of a gene of interest to provide an optimum sterical binding environment allowing the best modulation, preferably transcriptional activation, activity. In particular for CRISPR-based synthetic transcription factors according to the present invention acting together with a guide RNA as recognition moiety, the binding site can also reside in within the coding region of a gene of interest (downstream of the start codon of a gene of interest).

In further embodiments of the synthetic transcription factors of the present invention, the recognition domain can bind to the 5′ and/or 3′ untranslated region (UTR) of a gene of interest. In embodiments, where different recognition domains are employed, the at least two recognition domains can bind to different target regions of a morphogenic gene of interest, including 5′ and/or 3′UTRs, but they can also bind outside the gene region, but still in a certain distance of at most 1 to 1.500 bps thereto. One preferred region, where a recognition domain can bind, resides about −4 bp to about −300, preferably about −40 bp to about −170 bp upstream of the start codon of a morphogenic gene of interest. Notably, there is more recognition site flexibility for certain STFs disclosed herein, in particular for CRISPR-based STFs due to the additional functions of at least one gRNA in said STFs.

According to the various aspects and embodiments presented herein, the length of a recognition domain and thus the corresponding recognition site in a genome of interest may thus vary depending on the STF and the nature of the recognition domain applied. Based on the molecular characteristics of the at least one recognition domain, this will also determine the length of the corresponding at least one recognition site. For example, where individual zinc finger may be from about 8 bp to about 20 bp, wherein arrays of between three to six zinc finger motifs may be preferred, individual TALE recognition sites may be from about 11 to about 30 bp, or more. Recognition sites of gRNAs of a CRISPR-based STF comprise the targeting or “spacer” sequence of a gRNA hybridizing to a genomic region of interest, whereas the gRNA comprises further domains, including a domain interacting with a disarmed CRISPR effector according to the present disclosure. The recognition site of a STF based on a disarmed CRISPR effector will comprise a PAM motif, as the PAM sequence is necessary for target binding of any CRISPR effector and the exact sequence is dependent upon the species of the CRISPR effector, i.e., a disarmed CRISPR effector as disclosed herein.

In one embodiment of the various aspects of the present invention, the synthetic transcription factor may comprise at least one activation domain, wherein the at least one activation domain may be selected from the group consisting of an acidic transcriptional activation domain, preferably, wherein the at least one activation domain may be from an avirulence gene of Xanthomonas oryzae, VP16 or tetrameric VP64 from Herpes simplex, VPR, SAM, Scaffold, Suntag, P300, VP160, or any combination thereof. To enhance modulation of at least one morphogenic gene of interest, two, three, four, five, or more than five activation domains may be present. In a preferred embodiment of the present invention, the activation domain is VPR (SEQ ID NO: 276).

VP16 is a transcription factor originally found in herpes simplex virus (HSV) type 1 that is involved in the activation of the viral immediate-early genes (Flint and Shenk, 1997; Wysocka and Herr, 2003). The VP16 wild-type sequence has 490 amino acids with a core domain in its central region required for indirect DNA binding and a carboxy-terminal TAD located within its last 81 amino acids (Greaves and O'Hare, 1989; Triezenberg et al., 1988). VP16 is originally contained within the virion (virus particle) of the HSV and released into animal cells upon infection. VP16 first binds to the host nuclear protein HCF through its core domain and subsequently binds to another host nuclear protein Oct-1 to form a three-component protein complex. This complex then binds to its target DNA sequence TAATGARAT (R is a purine) in the promoters of immediate-early genes. This is achieved through interactions between Oct-1 and the target DNA sequence or a consensus octamer motif that overlaps the 5′ portion of this sequence. HCF then stabilizes the interaction between VP16 and Oct1. Once recruited to immediate-early genes, VP16 activates genes through interactions between the TAD and other transcription factors (Hirai et al., Int. J. Dev. Biol., 2010, 54(11-12):1589-1596). Meanwhile, the original VP16 domain has been extensively exploited for a variety of studies using artificial or synthetic transcription factors. Usually, a core domain comprising the minimal activation domain of VP16 in single form, or as, for example, triple (VP48) or as 10× tandem copies of VP16 (VP160) is used for these purposes.

The natural activation domain of the TAL effector genes of Xanthomonas oryzae is the most obvious activation domain for use with TAL transcription factors, and also represents one activation domain, which can be used, alone or in combination, according to the various aspects of the present invention, but have been used in other settings as well. They belong to a family of acidic (transcriptional) activation domains.

The SAM (synergistic activation mediator) activation domain usually consists of three components: a nucleolytically inactive/inactivated CRISPR nuclease, usually in combination with a VP64 fusion, a guide RNA incorporating two MS2 RNA aptamers at the tetraloop and stem-loop, and the MS2-P65-HSF1 activation helper protein (Konermann et al., 2015, “Genome-scale transcriptional activation by an engineered CRISPR-Cas9 complex”. Nature 517:583-588). Therefore, the guide RNA may contain two copies of an RNA hairpin from the MS2 bacteriophage, which interacts with the RNA-binding protein (RBP) MCP (MS2 coat protein).

The SAM system employs multiple transcriptional activators to create a synergistic effect, which makes the SAM system a highly versatile activation domain used alone, or in combination with further activation domains for the synthetic transcription factors according to the present invention. In a preferred embodiment, wherein the synthetic transcription factor uses a CRISPR-based recognition domain, the guide RNA can be further engineered to optimize the interplay between the activation and the recognition domain.

A further activation domain to be used alone or in combination according to the present invention is the tripartite effector VPR (VP64, p65, and Rta) fused to a recognition domain of interest linked in tandem (Russa and Qi, Mol. Cell. Biol. 2015 November; 35(22): 3800-3809). Use of a VPR activation domain was shown to result in over 20-fold of transcriptional activation of GFP expression in mammalian cells (Liu et al. (2017), Engineering cell signaling using tunable CRISPR/Cpf1 based transcription factors. Nature Communications, 8(1):2095).

Yet a further activation domain to be used alone or in combination according to the present invention is “scaffold” recruiting multiple copies of, e.g., VP64, to a special guide RNA, optionally together with further activators (Chavez et al., Nat. Methods, 2016, 13(7), 563-567).

Another activation domain to be used alone or in combination according to the present invention is “Suntag” comprising a repeating peptide array, which can recruit multiple copies of an antibody-fusion protein to create a potent synthetic transcription factor by recruiting multiple copies of a transcriptional activation domain to a nuclease-deficient recognition domain of a synthetic transcription factor of the present invention (Tanenbaum et al., Cell, 2014, 159(3):635-46).

In another embodiment, the SAM activation domain system may be employed to, in particular a SAM-modified guide RNA, together with a suntag activation domain to simultaneously recruit both a single-chain variable fragment (scFv) with a desired specificity, coupled to, for example VP64, to one end of a recognition domain, and p65-hsfI to the guide RNA for CRISPR-based synthetic transcription factors. The scFvs, not representing activators per se, with their extremely high specificity and versatility of target recognition, which can be engineered, are thus highly suitable to recruit multiple copies of an activator of interest to a position of interest, i.e., the scFv can be used as amplifier according to the various aspects and embodiments of the present invention together with an activation domain as disclose herein.

Yet another activation domain to be used alone or in combination according to the present invention is p300 or EP300 or E1A (used interchangeably herein), or CBP (also known as CREB-binding protein or CREBBP). Both p300 and CBP interact with numerous transcription factors and act to increase the expression of their target genes (Kasper et al., 2006, Mol. Cell. Biol., 26(3), 789-809). P300 and CBP have similar structures. Both contain five protein interaction domains: the nuclear receptor interaction domain (RID), the KIX domain (CREB and MYB interaction domain), the cysteine/histidine regions (TAZ1/CH1 and TAZ2/CH3) and the interferon response binding domain (IBiD). The last four domains, KIX, TAZ1, TAZ2 and IBiD of p300, each bind tightly to a sequence spanning both transactivation domains 9aaTADs of transcription factor p53. In addition, p300 and CBP each contain a protein or histone acetyltransferase (PAT/HAT) domain and a bromodomain that binds acetylated lysines and a PHD finger motif with unknown function. The conserved domains are connected by long stretches of unstructured linkers. P300 and CBP may increase gene expression in three ways: by relaxing the chromatin structure at the gene promoter through their intrinsic histone acetyltransferase (HAT) activity; by recruiting the basal transcriptional machinery including RNA polymerase II to the promoter; and/or by acting as adaptor molecules.

According to the various embodiments of the present invention, the at least one recognition domain and the at least one activation domain of the synthetic transcription factor of the present invention may be individually optimized to allow a perfect binding and modulation activity. Therefore, a specific number of activation domains may be suitable for a given recognition domain, properly positioned in the synthetic transcription factor construct, to allow optimum modulation activity, preferably transcriptional activation. Therefore, the at least one activation domain according to the various aspects of the present invention may comprise certain modifications to optimize the at least one activation domain to interact with the at least one recognition domain in an optimum way so that both domains have access to a target site of interest to be modulated.

In one embodiment, the at least one activation domain may be located N-terminal and/or C-terminal relative to the at least one recognition domain within a synthetic transcription factor of the present invention. This configuration can be the best configuration for fusion molecules between at least one recognition domain and at least one activation domain. According to various embodiments, the at least one recognition domain and the at least one activation domain may be separated by a suitable linker sequence to allow optimum flexibility and to avoid sterical hindrance of the domains to fulfill their functions.

In one embodiment, the synthetic transcription factor may comprise at least one further element, including at least one nuclear localization signal (NLS), an organelle localization signal, including, for example, a mitochondrion localization signal or a chloroplast localization signal to target the STF to a compartment within a cell or cellular system, where the STF can exert its function. Furthermore, the synthetic transcription factor may comprise at least one tag, e.g. to visualize the synthetic transcription factor, to track the subcellular localization of the transcription factor and/or to provide a active moiety within the synthetic transcription factor, e.g. a scFv binding site, to attach further molecules to the synthetic transcription factor, a translocation domain, e.g. a translocation domain as present in TALE molecules, and the like as further disclosed herein, and as known to the skilled person. The at least one further domain may be positioned N-terminal and/or C-terminal relative to the at least one recognition domain, including a positioning between the at least one recognition and the at least one activation domain, e.g. at least one NLS may be positioned between one recognition domain and another recognition domain and/or an activation domain. If provided as a transcribable/translatable vector, the STF may comprise at least one promoter for optimum transcription within a target cell or cellular system of interest. The skilled person is able to define suitable promoters, preferably strong promoters, either with inducible or constitutive expression, depending on a cellular system of interest. An example for a very strong constitutive promoter in the plant system, e.g., Zea mays, is BdUbi10. A weaker promoter would be the BdEF1 for example. Inducible plant promoters are the tetracycline-, the dexamethasone-, and salicylic acid inducible promoters. Other promoters suitable according to the present invention are a CaMV (Cauliflower mosaic virus) 35S or a double 35S promoter. Other constitutive eukaryotic promoters are CMV (Cytomegalovirus), EF1a, TEF1, SV40, PGK1 (human or mouse), Ubc (ubiquitin 1), human beta-actin, GDS, GAL1 or 2 (for a yeast system), CAG (comprising a CMV enhancer, chicken beta actin promoter, and rabbit beta-globin splice acceptor), H1, or U6. A variety of inducible promoters is known to the skilled person.

Therefore, a variety of different architectures can be present in the STFs according to the present invention. As the STFs of the present application have a modular character, several STFs with a different domain architecture can be designed for a given target and can be evaluated in a comparative way in vitro to deduce the architecture providing the best modulation effect.

In one embodiment of the present invention, the STF comprises a N-terminal TAL recognition domain and a C-terminal VP64 activation domain, wherein the STF further comprises a SV40 nuclear localization signal (NLS) between the N-terminal recognition domain and the C-terminal activation domain.

In yet another embodiment of the present invention, the STF comprises a N-terminal CRISPR/dCas9 or CRISPR/dCpf1 recognition domain and a C-terminal VP64 activation domain associated with a SV40 nuclear localization signal (NLS) at its C-terminus, wherein the STF further comprises two SV40 NLSs between the N-terminal recognition domain and the C-terminal activation domain.

In a preferred embodiment of the various aspects of the present invention, the recognition domain of the STF is or is a fragment of at least one disarmed CRISPR/Cpf1 system and the activation domain is a VPR domain (SEQ ID NO: 276), optionally with a linker inbetween the recognition domain and the activation domain, preferably a 5×GS linker (SEQ ID NO: 277). In a further preferred embodiment of the various aspects of the present invention, the recognition domain of the STF comprises a disarmed LbCpf1 domain (SEQ ID NO: 282) a disarmed LbCpf1_RR domain (SEQ ID NO: 283) and/or a disarmed LbCpf1_RVR domain (SEQ ID NO: 284). To increase the efficiency of transcriptional regulation, preferably activation, gRNAs of the CRISPR/Cpf1 system are preferred which target a region up to 250 bp upstream of the transcription start site. In one embodiment of the herein described aspects of the invention, preferred gRNAs target a region within a range of 1-250, 1-200, 1-150, 1-100, 1-50, 50-250, 100-250, 150-250 or 200-250 bp upstream of the transcription start site, or any range in between the herein disclosed ranges.

In certain embodiments, the STFs, or the sequences encoding the same, according to the present invention can be provided as multiplex systems to target more than one gene of interest. For example, TALE and disarmed CRISPR-based STFs can be designed enabling the targeting of 2 to 7, or more, genetic loci of interests, or enabling the targeting of one gene of interest using two or more different STFs specifically designed to modulate said one gene of interest, by providing multiplex vectors, or by providing in vitro assembled multiplex STFs to be transformed or transfected in a cell or cellular system of interest.

In one embodiment, the synthetic transcription factor of the present invention, or the sequence encoding the same, may comprise at least one non-naturally occurring nucleotide, amino acid or synthetic sequence, or a combination thereof, covalently or non-covalently attached to at least one amino acid sequence of the synthetic transcription factor. This embodiment is particularly suitable in case that the synthetic transcription factor is delivered as pre-assembled complex into a cellular system of interest, and in particular for disarmed CRISPR-based synthetic transcription factors, wherein the recognition domain additionally comprises a gRNA component. As the ribonucleic acid is rather unstable, the gRNA recognition portion may be stabilized by a non-naturally occurring moiety, for example, a phosphorothioate backbone, or any other stabilizing nucleotide. Furthermore, the synthetic transcription factor, preferably in embodiments, wherein a pre-assembled protein complex is delivered into a cell or cellular system of interest, may comprise chemical modifications to stabilize, derivatize or functionalize the complex and/or to add at least one DNA repair template to the complex for embodiments aiming at a method for modifying the genetic material of a cellular system in a targeted way.

A challenge for any CRISPR-based approach is the fact that the RNA portion (gRNA) and the respective CRISPR polypeptide have to be transported to the nucleus or any other compartment comprising genomic DNA, i.e. the DNA target sequence, in a functional (not degraded) way. As RNA is less stable than a polypeptide or double-stranded DNA and has a higher turnover, especially as it can be easily degraded by nucleases, in some embodiments, a CRISPR RNA sequence and/or the DNA repair template nucleic acid sequence, if present in certain embodiments of the present invention, comprises at least one non-naturally occurring nucleotide. Preferred backbone modifications according to the present invention increasing the stability of the CRISPR RNA and/or increasing the stability of a DNA repair template nucleic acid sequence, if present, are selected from the group consisting of a phosphorothioate modification, a methyl phosphonate modification, a locked nucleic acid modification, an O-(2-methoxyethyl) modification, a di-phosphorothioate modification, and a peptide nucleic acid modification. Notably, all said backbone modifications still allow the formation of complementary base pairing between two nucleic acid strands, yet are more resistant to cleavage by endogenous nucleases. Depending on the disarmed CRISPR effector utilized in combination with a RNA/DNA nucleic acid sequence according to the present invention, it might be necessary not to modify those nucleotide positions of a CRISPR nucleic acid sequence, which are involved in sequence-independent interaction with the CRISPR polypeptide. Said information can be derived from the available structural information as available for CRISPR nuclease/CRISPR nucleic acid sequence complexes and for disarmed CRISPR effectors, e.g. dCas9.

In certain embodiments of the present invention, it is envisaged that at least one CRISPR nucleic acid sequence (gRNA) and/or at least one optionally present DNA repair template nucleic acid sequence may comprise a nucleotide and/or base modification, preferably at selected, not all, nucleotide sequence positions. These modifications are selected from the group consisting of addition of acridine, amine, biotin, cascade blue, cholesterol, Cy3, Cy5, Cy5.5, Daboyl, digoxigenin, dinitrophenyl, Edans, 6-FAM, fluorescein, 3′-glyceryl, HEX, IRD-700, IRD-800, JOE, phosphate psoralen, rhodamine, ROX, thiol (SH), spacers, TAMRA, TET, AMCA-S″, SE, BODIPY®, Marina Blue®, Pacific Blue®, Oregon Green®, Rhodamine Green®, Rhodamine Red®, Rhodol Green® and Texas Red®. Preferably, said additions are incorporated at the 3′ or the 5′ end of the CRISPR nucleic acid sequence and/or the DNA repair template nucleic acid sequence. This modification has the advantageous effects, that the cellular localization of the CRISPR nucleic acid sequence and/or the optionally present DNA repair template nucleic acid sequence within a cell can be visualized to study the distribution, concentration and/or availability of the respective sequence. Furthermore, the interaction of the synthetic transcription factor of interest and the binding behavior can be studied. Methods of studying such interactions or for visualization of a nucleotide sequence modified or tagged as detailed above are available to the skilled person in the respective field.

In one embodiment, any nucleotide of the at least one CRISPR nucleic acid sequence or any other component of the sequence encoding at least one synthetic transcription factor of the present invention can comprise one of the above modifications as a label or linker. As used herein, “nucleotide” can thus generally refer to a base-sugar-phosphate combination. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dTTP, dUTP, dGTP, dTTP, or derivatives thereof. Such derivatives can include, for example and not limitation, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited to fluorescein, 5-carboxyfluorescein (FAM), 2′7′-5 dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS).

Labels or linkers can also comprise moieties suitable for click chemistry to link the at least one CRISPR guide nucleic acid sequence or a portion thereof and/or a DNA repair template nucleic acid sequence and/or at least one recognition domain of a synthetic transcription factor and/or at least one activation domain of a synthetic transcription factor to each other.

Of the reactions comprising the click chemistry field suitable to modify any nucleic acid or amino acid according to the present invention to build a molecular complex, in vitro or in vivo, one example is the Huisgen 1,3-dipolar cycloaddition of alkynes to azides to form 1,4-disubstituted-1,2,3-triazoles. The copper (I)-catalyzed reaction is mild and very efficient, requiring no protecting groups, and requiring no purification in many cases. The azide and alkyne functional groups are generally inert to biological molecules and aqueous environments. The triazole has similarities to the ubiquitous amide moiety found in nature, but unlike amides, is not susceptible to cleavage. Additionally, they are nearly impossible to oxidize or reduce.

As it is known to the skilled person, certain click chemistry reactions suitable for in vivo reactions rely on reactive groups, such as azides, terminal alkynes or strained alkynes (e.g., dibenzocyclooctyl (DBCO)), which reactive groups can be introduced into any form of RNA or DNA via accordingly modified nucleotides that are incorporated instead of their natural counterparts. Labels can be introduced enzymatically or chemically. The resulting CLICK-functionalized DNA can subsequently be processed via Cu(I)-catalyzed alkyne-azide (CuAAC) or Cu(I)-free strained alkyne-azide (SPAAC) click chemistry reactions, wherein copper-free reactions are preferable for applications within a cell or living system. These reactions can be used according to the present invention to introduce a biotin group for subsequent purification tasks (via azides, alkynes of biotin or DBCO-containing biotinylation reagents), to introduce a fluorescent group for subsequent microscopic imaging (via fluorescent azides, fluorescent alkynes or DBCO-containing fluorescent dyes), or to crosslink to biomolecules, e.g., the at least one domain of, or the at least one synthetic transcription factor of the present invention, and optionally a DNA repair template, if present, to covalently link and/or provide functionalized biomolecules.

In one embodiment, an optionally purified and functionally associated 5′ or 3′ end click-chemistry-labeled CRISPR nucleic acid sequence according to the present invention may be delivered by any transformation or transfection method to a cell or cell system stably or transiently expressing a corresponding disarmed CRISPR polypeptide. Thereby, as the CRISPR nucleic acid sequence interacts with and thereby directs the CRISPR polypeptide to act as a recognition domain according to the present invention. This allows the activation domain to precisely modulate the expression of at least one morphogenic gene of interest.

A variety of further chemical reactions and the corresponding modifications are available to the skilled person to link to nucleic acids according to the present disclosure to each other, or to any amino acid recognition and/or activation domain in a covalent way. These modifications include a variety of crosslinkers, such as thiol modifications, like a thioctic acid N-hydroxysuccinimide (NHS) ester, chemical groups that react with primary amines (—NH2). These primary amines are positively charged at physiologic pH; therefore, they occur predominantly on the outside surfaces of native protein tertiary structures where they are readily accessible to conjugation reagents introduced into the aqueous medium. Furthermore, among the available functional groups in typical biological or protein samples, primary amines are especially nucleophilic; this makes them easy to target for conjugation with several reactive groups. There are numerous synthetic chemical groups that will form chemical bonds with primary amines. These include isothiocyanates, isocyanates, acyl azides, NHS esters, sulfo-NHS esters containing a sulfonate (—SO3) group, for example, bis(sulfosuccinimidyl)suberate (BS3), sulfonyl chlorides, aldehydes, glyoxals, epoxides, oxiranes, carbonates, aryl halides, imidoesters, carbodiimides, such as, for example 1-Ethyl-3-(3-dimethylaminopropyl)carbodiimide (EDC) or dicyclohexylcarbodiimide (DCC), anhydrides, and fluorophenyl esters.

In certain embodiments, any nucleic acid sequences according to the various aspects of the present invention can be codon optimized to adapt the sequence for optimum performance in a target organism or cell of interest. For example, a sequence may be codon optimized to allow a high transcription rate in a plant cell of interest of a plant genus of interest, or the sequences may be codon optimized for use in a mammalian, e.g., a murine or human cell.

According to the various embodiments of the present invention, the synthetic transcription factor and/or the at least one recognition domain may comprise a sequence set forth in any one of SEQ ID NOs: 1 to 94, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 1 to 94, or wherein the synthetic transcription factor and/or at least one recognition domain, binds to a regulation region set forth in SEQ ID NOs: 95 to 190, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 95 to 190.

In one embodiment of the various aspects of the present invention, the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

Synthetic transcription activators according to the present invention, preferably specific for WUS and/or BBM, can be easily co-delivered with gene editing machineries and/or T-DNAs to improve transformation efficiencies in a plant cell and to induce regeneration of the transgenic plant. The present invention therefore further relates to methods for inducing regeneration of transformed plant cells by promoting the expression of growth-stimulating genes (morphogenic genes) such as, for example, BBM and WUS.

According to the various embodiments and aspects disclosed herein, the cellular system may be selected from the group consisting of at least one eukaryotic cell or eukaryotic organism, preferably wherein the at least one eukaryotic cell may be at least one plant cell, and/or wherein the at least one eukaryotic organism may be a plant or a part of a plant.

In certain embodiments disclosed herein, the cellular system to be modulated, transformed and/or transfected may be selected from the group consisting of at least one eukaryotic cell or eukaryotic organism, preferably wherein the at least one eukaryotic cell may be at least one plant cell, and/or wherein the at least one eukaryotic organism is a plant or a part of a plant.

In certain embodiments according to the various embodiments and aspects disclosed herein, the at least one part of the plant may be selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In embodiments, wherein the cellular system is, or originates from, a plant cell, the at least one plant or the at least one part of a plant may originate from a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In a further aspect of the present invention provides a method for increasing the transformation efficiency in a cellular system, wherein the method may comprise the steps of: (a) providing a cellular system; (b) introducing into the cellular system at least one synthetic transcription factor, or a nucleotide sequence encoding the same; and (c) introducing into the cellular system at least one nucleotide sequence of interest; (d) optionally: culturing the cellular system under conditions to obtain a transformed progeny of the cellular system; wherein the at least one synthetic transcription factor, or the nucleotide sequence encoding the same, comprises at least one recognition domain and at least one activation domain, wherein the synthetic transcription factor is configured to modulate the expression, preferably the transcription, of at least one morphogenic gene in the cellular system; and wherein the at least one synthetic transcription factor, or the nucleotide sequence encoding the same, is introduced in parallel to, or sequentially with the introduction of the at least one nucleotide sequence of interest.

The present invention therefore discloses methods of improving the efficiency of plant transformation or transfection and/or regeneration of plants by using synthetic transcription factors specific for endogenous morphogenic genes which can reprogram the cell and induce cell division in a large variety of plant species to provide reliable methods of transforming cellular systems, including those cellular systems known to be hard to modify and/or transform by currently available methods. In particular, certain elite lines comprising a highly valuable elite event (i.e., events very rarely achieved and, if at all, derived from an extraordinary and thus surprising event) and germplasm of said elite lines may be highly recalcitrant to in vitro culture and transformation attempts. Such genotypes usually do not produce an appropriate embryogenic or organogenic culture response on culture media developed to elicit such responses from typically suitable explants such as immature embryos. Furthermore, when exogenous DNA or other biomolecules are introduced into these immature embryos, no successful modification event may be recovered after cumbersome rounds of selection, or only so few events may be recovered as to make transformation of such a genotype impractical.

In one embodiment, the method may comprise that (a) the at least one synthetic transcription factor, or the sequence encoding the same, or at least one component of the at least one synthetic transcription factor, or the sequence encoding the same; and (b) the at least one nucleotide sequence of interest is/are introduced into the cellular system by means independently selected from biological and/or physical means, including transfection, transformation, including transformation by Agrobacterium spp., preferably, Agrobacterium tumefaciens, a viral vector, biolistic bombardment, transfection using chemical agents, including polyethylene glycol transfection, electro-poration, cell fusion or any combination thereof.

Therefore, an “introduction” or the process of “introducing” can comprise any biological, chemical and/or physical means of introducing or delivering a biomolecule into a cellular system of interest. Notably, any combination of introduction or delivery techniques may be applied. Furthermore, different components to be introduced into a cellular system of interest may be introduced by the same technique, simultaneously or subsequently, for example, by co-bombardment, or they may be introduced simultaneously or subsequently by different introduction techniques.

It has been demonstrated for the first time in the context of the present invention, that a Cpf1-based transcription regulation system is a powerful tool for transcriptional activation or suppression of endogenous target genes in plants and—as mentioned above—has several advantages over other systems. It can therefore be used for improving the efficiency of plant transformation or transfection and/or regeneration of plants by using synthetic transcription factors specific for endogenous morphogenic genes providing methods of transforming cellular systems, including those cellular systems known to be hard to modify and/or transform by currently available methods.

In a preferred embodiment of the method for increasing the transformation efficiency in a cellular system of the present invention, the at least one recognition domain is or is a fragment of at least one disarmed non-functional CRISPR/nuclease system.

In a further preferred embodiment of the method of the present invention, the at least one disarmed CRISPR/nuclease system is a CRISPR/dCpf1 system, wherein the at least one disarmed CRISPR/nuclease system comprises at least one guide RNA.

In one embodiment, the at least one activation domain of the at least one synthetic transcription factor is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof. Preferably, the activation domain is a VPR domain (SEQ ID NO: 276).

In another embodiment, the at least one activation domain of the at least one synthetic transcription factor is located N-terminal and/or C-terminal relative to the at least one recognition domain of the at least one synthetic transcription factor.

In a preferred embodiment of the method of the present invention, the recognition domain of the STF is or is a fragment of at least one disarmed CRISPR/Cpf1 system and the activation domain is a VPR domain, optionally with a linker inbetween the recognition domain and the activation domain, preferably a 5×GS linker.

The increase in transformation efficiency according to the various aspects and embodiments of the present invention can comprise any statistically significant increase when compared to a control plant or cellular system. For example, an increase in transformation efficiency can comprises about 0.2%, 0.5%, 5%, 10%, 15%, 20%, 25%, 30%, 35%, 40%, 45%, 50%, 55%, 60%, 65%, 70%, 75%, 80%, 85%, 90%, 95%, 120%, 125% or greater increase when compared to a control plant or a control plant part, or a control cellular system. Alternatively, the increase in transformation efficiency can include about a 0.2 fold, 0.5 fold, 1 fold, 2 fold, 4 fold, 8 fold, 16 fold, or 32 fold or greater increase in transformation efficiency in the plant, plant part or cellular system when compared to a control plant or plant part or cellular system.

In one embodiment, the methods of the present invention may comprise that the at least one nucleotide sequence of interest is provided as part of at least one vector, or as at least one linear molecule.

In one embodiment of the methods disclosed herein, the at least one nucleotide sequence of interest may be selected from the group consisting of a transgene, a modified endogenous gene, a synthetic sequence, an intronic sequence, a coding sequence or a regulatory sequence.

In one embodiment of the methods disclosed herein, the at least one nucleotide sequence of interest may be a transgene, wherein the transgene may comprise a nucleotide sequence encoding a gene of a genome of an organism of interest, or at least a part of said gene.

In one embodiment, a regulatory sequence according to the present invention may be a promoter sequence, wherein the editing or mutation or modulation of the promoter comprises replacing the promoter, or promoter fragment with a different promoter (also referred to as replacement promoter) or promoter fragment (also referred to as replacement promoter fragment), wherein the promoter replacement results in any one of the following or any one combination of the following: an increased promoter activity, an increased promoter tissue specificity, a decreased promoter activity, a decreased promoter tissue specificity, a new promoter activity, an inducible promoter activity, an extended window of gene expression, a modification of the timing or developmental progress of gene expression in the same cell layer or other cell layer, for example, extending the timing of gene expression in the tapetum of anthers, a mutation of DNA binding elements and/or a deletion or addition of DNA binding elements. The promoter (or promoter fragment) to be modified can be a promoter (or promoter fragment) that is endogenous, artificial, pre-existing, or transgenic to the cell that is being edited. The replacement promoter or fragment thereof can be a promoter or fragment thereof that is endogenous, artificial, pre-existing, or transgenic to the cell that is being edited. Any other regulatory sequence according to the present disclosure may be modified as detailed for a promoter or promoter fragment above.

Particularly in case of plant genomes to be modified, it may be desirable that the modification as mediated by the methods of the present invention does not result in a genetically modified organism by integrating foreign DNA into the parent genome in an imprecise way, as environmental, regulatory and political issues have to be concerned. Therefore, the embodiments according to the present invention providing methods for introducing a genetic material of interest in a cellular system in a transient way are particularly suitable for providing a cellular system comprising a modification at a predetermined location without inserting foreign DNA and thus without providing a cell or organism regarded as genetically modified organism, as all tools necessary to perform the methods of the present invention can be provided to the cellular system in a transient way in active form.

In one embodiment of the methods described herein, transcriptional activation is combined with modification of a plant genome in a fully transiently manner, thereby obtaining a plant organism comprising a modification at a predetermined genetic location without inserting foreign DNA into the plant genome and thus providing a plant organism which is not regarded as a genetically modified organism. The methods described herein therefore provide means to modify a plant genome which do not require labor-intensive deregulation procedures. In yet another embodiment of the methods described herein, the STFs and/or the site-specific nuclease are provided DNA-free, e.g. as protein or RNP, thereby providing a regulatory benefit. In one embodiment of the various methods disclosed herein, the methods may be performed in a fully transient way. In other embodiments, the methods may be performed by a combination of stable and transient approaches. In yet a further embodiment, the methods may also be performed by stably introducing suitable delivery tools to a cell or cellular system of interest.

In another embodiment of the various aspects of the present invention, the at least one nucleotide sequence of interest to be introduced into a cellular system may be a transgene of an organism of interest, wherein the transgene or part of the transgene may be selected from the group consisting of a gene encoding resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, resistance or tolerance to 2,4-D, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content.

In another embodiment of the various aspects of the present invention, the at least one nucleotide sequence of interest may be at least part of a modified endogenous gene of an organism of interest, wherein the modified endogenous gene may comprise at least one deletion, insertion and/or substitution of at least one nucleotide in comparison to the nucleotide sequence of the unmodified endogenous gene.

In yet a further embodiment of the various aspects of the present invention, the at least one nucleotide sequence of interest may be at least part of a modified endogenous gene of an organism of interest, wherein the modified endogenous gene may comprise at least one of a truncation, duplication, substitution and/or deletion of at least one nucleotide position encoding a domain of the modified endogenous gene.

In one embodiment, the at least one nucleotide sequence of interest may be at least part of a regulatory sequence, wherein the regulatory sequence may comprise at least one of a core promoter sequence, a proximal promoter sequence, a cis regulatory sequence, a trans regulatory sequence, a locus control sequence, an insulator sequence, a silencer sequence, an enhancer sequence, a terminator sequence, and/or any combination thereof.

Any synthetic transcription factor as disclosed herein below can be used for the different methods according to the present invention as mediator to specifically modulate the transcription of a morphogenic gene of interest. This modulation, preferably a transcriptional upregulation, allows a better transformation efficiency of a cellular system, preferably a plant or plant part of interest.

According to the various embodiments of the methods disclosed herein, the preferred morphogenic gene to be modulated may be selected from the group consisting of BBM, WUS, including WUS2, a WOX gene, a WUS or BBM homologue, Lec1, Lec2, WIND1, ESR1, PLT3, PLT5, PLT7, IPT, IPT2, Knotted1, and RKD4.

Preferably, the morphogenic gene comprises a nucleotide sequence selected from the group consisting of (i) a nucleotide sequence set forth in any one of SEQ ID NOs: 199 to 237, (ii) a nucleotide sequence having the coding sequences of the nucleotide sequence set forth in any one of SEQ ID NOs: 199 to 237, (iii) a nucleotide sequence complementary to the nucleotide sequence of (i) or (ii), (iv) a nucleotide sequence having at least 50%, 60%, 70%, 75%, 80%, 85%, 90%, 91%, 92%, 93%, 94%, 95%, 96%, 97%, 98%, or 99% identity, preferably over the whole length, to the the nucleotide sequence of (i), (ii) or (iii), (v) a nucleotide sequence hybridzing the nucleotide sequence of (iii) under stringent conditions, (vi) a nucleotide sequence encoding a protein comprising the amino acid sequence set forth in any one of SEQ ID NOs: 238 to 258, (vii) a nucleotide sequence encoding a protein comprising the amino acid sequence at least 50%, 60%, 70%, 75%, 80%, 85%, 90%, 91%, 92%, 93%, 94%, 95%, 96%, 97%, 98%, or 99% identical to a sequence set forth in any one of SEQ ID NOs: 238 to 258, or (viii) a nucleotide sequence encoding a homologue, analogue or orthologue of protein comprising the amino acid sequence set forth in any one of SEQ ID NOs: 238 to 258.

In certain embodiments, the synthetic transcription factor used in the methods of the present invention may be configured to modulate expression, preferably transcription, of the morphogenic gene by binding to a regulation region located at a certain distance in relation to the start codon.

In certain embodiments, the synthetic transcription factor and/or the at least one recognition domain used in the methods of the present invention may comprise a sequence set forth in any one of SEQ ID Nos: 1 to 94, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 1 to 94, or wherein the synthetic transcription factor and/or at least one recognition domain, binds to a regulation region set forth in SEQ ID NOs: 95 to 190 or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 95 to 190.

In one embodiment of the methods of the present invention, the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

In certain embodiments of the methods of the present invention, the cellular system may be selected from the group consisting of at least one eukaryotic cell or eukaryotic organism, preferably wherein the at least one eukaryotic cell may be at least one plant cell, and/or wherein the at least one eukaryotic organism is a plant or a part of a plant.

In other embodiments of the methods of the present invention, the at least one part of the plant may be selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In further embodiments of the methods of the present invention, the at least one plant cell, the at least one plant or the at least one part of a plant may originate from a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In a further aspect of the present invention, independently or together with the further aspects and embodiments disclosed herein, provides a method of modifying the genetic material of a cellular system at a predetermined location, wherein the method may comprise the following steps: (a) providing a cellular system; (b) introducing at least one synthetic transcription factor, or a sequence encoding the same, into the cellular system, (c) further introducing into the cellular system (i) at least one site-specific nuclease, or a sequence encoding the same, wherein the site-specific nuclease induces a double-strand break at the predetermined location; (ii) optionally: at least one nucleotide sequence of interest, preferably flanked by one or more homology sequence(s) complementary to one or more nucleotide sequence(s) adjacent to the predetermined location in the genetic material of the cellular system; and; (e) optionally: determining the presence of the modification at the predetermined location in the genetic material of the cellular system; and (f) obtaining a cellular system comprising a modification at the predetermined location of the genetic material of the cellular system; wherein the at least one synthetic transcription factor, or the nucleotide sequence encoding the same, comprises at least one recognition domain and at least one activation domain, wherein the at least one synthetic transcription factor is configured to modulate the expression, preferably the transcription, of at least one morphogenic gene in the cellular system; and wherein the at least one synthetic transcription factor, or the nucleotide sequence encoding the same, may be introduced in parallel to, or sequentially with the introduction of the at least one site-specific nuclease, or the sequence encoding the same and the optional at least one nucleotide sequence of interest.

This aspect and the associated embodiments thus synergistically combine the advantages of the targeted modulation of the transcription rate of at least one morphogenic gene of interest in a cellular system with a highly site-directed genome editing (GE) method of introducing certain effectors into the cell. By providing an environment within a cellular system comprising at least one synthetic transcription factor according to the present invention, it is thus possible to specifically modulate the transcription of at least one morphogenic gene in the cellular system before or simultaneously with the introduction of at least one site-specific nuclease (SSN), i.e., an enzyme comprising DNA double-strand, or DNA single-strand cleavage capability, or a sequence encoding the same, and optionally further tools like repair templates (RTs) to provide an environment, wherein the cellular system is highly transformation competent and further possesses a high regeneration capability. These factors guarantee a successful editing and regeneration of the such edited genetic material within a cellular system of interest and further allows regenerating a plant or plant material from the modified cellular system, as the cellular system is much more tolerant and viable during the GE event based on the co- or pre-treatment with at least one synthetic transcription factor, or a sequence encoding the same.

In one embodiment, the method further comprises the step of culturing the cellular system under conditions to obtain a genetically modified progeny of the modified cellular system.

The term “adjacent” or “adjacent to” as used herein in the context of the predetermined location and the one or more homology region(s) may comprise an upstream and a downstream adjacent region, or both. Therefore, the adjacent region is determined based on the genetic material of a cellular system to be modified, said material comprising the predetermined location.

There may be an upstream and/or downstream adjacent region near the predetermined location. For site-specific nucleases (SSNs) inducing blunt double-strand breaks (DSBs), the “predetermined location” will represent the site the DSB is induced within the genetic material in a cellular system of interest. For SSNs leaving overhangs after DSB induction, the predetermined location means the region between the cut in the 5′ end on one strand and the 3′ end on the other strand. The adjacent regions in the case of sticky end SSNs thus may be calculated using the two different DNA strands as reference. The term “adjacent to a predetermined location” thus may imply the upstream and/or downstream nucleotide positions in a genetic material to be modified, wherein the adjacent region is defined based on the genetic material of a cellular system before inducing a DSB or modification. Based on the different mechanisms of SSNs inducing DSBs, the “predetermined location” meaning the location a modification is made in a genetic material of interest may thus imply one specific position on the same strand for blunt DSBs, or the region on different strands between two cut sites for sticky cutting DSBs, or for nickases used as SSNs between the cut at the 5′ position in one strand and at the 3′ position in the other strand.

If present, the upstream adjacent region defines the region directly upstream of the 5′ end of the cutting site of a site-specific nuclease of interest with reference to a predetermined location before initiating a double-strand break, e.g., during targeted genome engineering. Correspondingly, a downstream adjacent region defines the region directly downstream of the 3′ end of the cutting site of a SSN of interest with reference to a predetermined location before initiating a double-strand break, e.g., during targeted genome engineering. The 5′ end and the 3′ end can be the same, depending on the site-specific nuclease of interest.

In certain embodiments, it may also be favorable to design at least one homology region in a distance away from the DSB to be induced, i.e., not directly flanking the predetermined location/the DSB site. In this scenario, the genomic sequence between the predetermined location and the homology sequence (the homology arm) would be “deleted” after homologous recombination had occurred, which may be preferred for certain strategies as this allows the targeted deletion of sequences near the DSB. Different kinds of RT configuration and design are thus contemplated according to the present invention for those embodiments relying on a RT. RTs may be used to introduce site-specific mutations, or RTs may be used for the site-specific integration of nucleic acid sequences of interest, or RTs may be used to assist a targeted deletion.

A “homology sequence(s)” introduced and the corresponding “adjacent region(s)” can each have varying and different length from about 15 bp to about 15.000 bp, i.e., an upstream homology region can have a different length in comparison to a downstream homology region. Only one homology region may be present. There is no real upper limit for the length of the homology region(s), which length is rather dictated by practical and technical issues. According to certain embodiments, depending on the nature of the RT and the targeted modification to be introduced, asymmetric homology regions may be preferred, i.e., homology regions, wherein the upstream and downstream flanking regions have varying length. In certain embodiments, only one upstream and downstream flanking region may be present.

In one embodiment according to the methods of the present invention, the at least one site-specific nuclease may comprise a zinc-finger nuclease, a transcription activator-like effector nuclease, a CRISPR/Cas system, including a CRISPR/Cas9 system, a CRISPR/Cfp1 system, a CRISPR/CasX system, a CRISPR/CasY system, an engineered homing endonuclease, and a meganuclease, and/or any combination, variant, or catalytically active fragment thereof.

Once expressed, the Cas9 protein and the gRNA form a ribonucleoprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex will bind any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA will continue to anneal to the target DNA in a 3′ to 5′ direction (relative to the polarity of the gRNA).

CRISPR/Cas, e.g. CRISPR/Cas9, and likewise CRISPR/Cpf1 or CRISPR/CasX or CRISPR/CasY and other CRISPR systems are highly specific when gRNAs are designed correctly, but especially specificity is still a major concern, particularly for clinical uses or targeted plant GE based on the CRISPR technology. The specificity of the CRISPR system is determined in large part by how specific the gRNA targeting sequence is for the genomic target compared to the rest of the genome. Therefore, the methods according to the present invention when combined with the use of at least one CRISPR nuclease as site-specific nuclease and further combined with the use of a suitable CRISPR nucleic acid can provide a significantly more predictable outcome of GE. Whereas the CRISPR complex can mediate a highly precise cut of a genome or genetic material of a cell or cellular system at a specific site, the methods presented herein provide an additional control mechanism guaranteeing a programmable and predictable repair mechanism.

According to the various embodiments of the present invention, the above disclosure with respect to covalent and non-covalent association or attachment also applies for CRISPR nucleic acid sequences, which may comprise more than one portion, for example, a crRNA and a tracrRNA portion, which may be associated with each other as detailed above. In one embodiment, a RT nucleic acid sequence of the present invention may be placed within a CRISPR nucleic acid sequence of interest to form a hybrid nucleic acid sequence according to the present invention, which hybrid may be formed by covalent and non-covalent association.

In yet a further embodiment according to the various aspects of the present invention, the one or more nucleic acid sequence(s) flanking the at least one nucleic acid sequence of interest at the predetermined location may have at least 85%-100% complementarity to the one or more nucleic acid sequence(s) adjacent to the predetermined location, upstream and/or downstream from the predetermined location, over the entire length of the respective adjacent region(s).

Notably, a lower degree of homology or complementarity of the at least one flanking region may be used, e.g. at least 70%, at least 75%, at least 80%, at least 81%, at least 82%, at least 83%, or at least 84% homology/complementarity to at least one adjacent region in the genetic material of interest. For high precision GE relying on HDR template, i.e., a RT as disclosed herein, more than 95% homology/complementarity are favorable to achieve a highly targeted repair event. As shown in Rubnitz et al., Mol. Cell Biol., 1984, 4(11), 2253-2258, also very low sequence homology might suffice to obtain a homologous recombination. As it is known to the skilled person, the degree of complementarity will depend on the genetic material to be modified, the nature of the planned edit, the complexity and size of a genome, the number of potential off-target sites, the genetic background and the environment within a cell or cellular system to be modified.

In one embodiment, the method further comprises the step of culturing the cellular system under conditions to obtain a genetically modified progeny of the modified cellular system.

In yet a further embodiment according to the various aspects of the present invention, the genetic material of the cellular system may be selected from the group consisting of a protoplast, a viral genome transferred in a recombinant host cell, a eukaryotic cell, tissue, or organ, preferably a plant cell, plant tissue or plant organ, and a eukaryotic organism, preferably a plant organism.

In one embodiment of the methods of the present invention, (i) the at least one synthetic transcription factor, or the sequence encoding the same, or at least one component of the at least one synthetic transcription factor, or the sequence encoding the same; and (ii) the at least one site-specific nuclease, or the sequence including the same; and optionally (iii) the at least one nucleotide sequence of interest may be introduced into the cellular system by means independently selected from biological and/or physical means, including transfection, transformation, including transformation by Agrobacterium spp. transformation, preferably by Agrobacterium tumefaciens, a viral vector, biolistic bombardment, transfection using chemical agents, including polyethylene glycol transfection, or any combination thereof.

In one embodiment of the methods for modifying the genetic material of a cellular system at a predetermined location of the present invention, the at least one recognition domain may be or may be a fragment of a molecule selected from the group consisting of at least one TAL effector, at least one disarmed CRISPR/nuclease system, at least one Zinc-finger domain, and at least one disarmed homing endonuclease, or any combination thereof.

In one embodiment of the methods for modifying the genetic material of a cellular system at a predetermined location of the present invention, the at least one disarmed CRISPR/nuclease system may be selected from a CRISPR/dCas9 system, a CRISPR/dCpf1 system, a CRISPR/dCasX system or a CRISPR/dCasY system, or any combination thereof, wherein the at least one disarmed CRISPR/nuclease system may comprise at least one guide RNA, preferably a guide RNA optimized for the specific disarmed CRISPR/nuclease system and the specific target site within or near a morphogenic system to increase the recognition and/or binding properties of the synthetic transcription factor of the present invention.

In a preferred embodiment of the methods for modifying the genetic material of a cellular system at a predetermined location of the present invention, the at least one recognition domain is or is a fragment of least one disarmed CRISPR/nuclease system.

Due to the advantages described above, it is particularly preferred, that in the methods for modifying the genetic material of a cellular system at a predetermined location of the present invention, the at least one disarmed CRISPR/nuclease system is a CRISPR/dCpf1 system, wherein the at least one disarmed CRISPR/nuclease system comprises at least one guide RNA.

In a further embodiment of the methods for modifying the genetic material of a cellular system at a predetermined location of the present invention, the at least one activation domain of the at least one synthetic transcription factor may be selected from the group consisting of an acidic transcriptional activation domain, preferably, wherein the at least one activation domain may be from an avirulence gene of Xanthomonas oryzae, VP16 or tetrameric VP64 from Herpes simplex, VPR, SAM, Scaffold, Suntag, P300, VP160, or any combination thereof. In a preferred embodiment of the methods for modifying the genetic material of a cellular system at a predetermined location of the present invention, the at least one activation domain is VPR (SEQ ID NO: 276). In a further preferred embodiment of the present invention, a combination of different activation domains can be used, e.g. VP64-p65-Rita or any combination of activation domains commonly known in the art.

Suitable linkers for the herein described CRISPR/Cpf1 systems comprise flexible linkers, such as 5GS or XTEN, while in vivo cleavable linkers are not suitable for the herein described aspects of the invention.

To increase the efficiency of transcriptional regulation, preferably activation, gRNAs of the CRISPR/Cpf1 system are preferred which target a region up to 250 bp upstream of the transcription start site. In one embodiment of the herein described aspects of the invention, preferred gRNAs target a region within a range of 1-250, 1-200, 1-150, 1-100, 1-50, 50-250, 100-250, 150-250 or 200-250 bp upstream of the transcription start site, or any range in between the herein disclosed ranges.

In another embodiment of the methods for modifying the genetic material of a cellular system at a predetermined location of the present invention, the at least one activation domain of the at least one synthetic transcription factor may be located N-terminal and/or C-terminal relative to the at least one recognition domain of the at least one synthetic transcription factor.

In a preferred embodiment of the method for modifying the genetic material of a cellular system of the present invention, the recognition domain of the STF is or is a fragment of at least one disarmed CRISPR/Cpf1 system and the activation domain is a VPR domain, optionally with a linker inbetween the recognition domain and the activation domain, preferably a 5×GS linker.

In yet a further embodiment of the methods for modifying the genetic material of a cellular system at a predetermined location of the present invention, the at least one morphogenic gene may be selected from the group consisting of BBM, WUS, including WUS2, a WOX gene, a WUS or BBM homologue, Lec1, Lec2, WIND1, ESR1, PLT3, PLT5, PLT7, IPT, IPT2, Knotted1, and RKD4.

In a further embodiment, there is provided the methods for modifying the genetic material of a cellular system at a predetermined location of the present invention, wherein the at least one morphogenic gene comprises a nucleotide sequence selected from the group consisting of (i) a nucleotide sequence set forth in any one of SEQ ID NOs: 199 to 237, (ii) a nucleotide sequence having the coding sequences of the nucleotide sequence set forth in any one of SEQ ID NOs: 199 to 237, (iii) a nucleotide sequence complementary to the nucleotide sequence of (i) or (ii), (iv) a nucleotide sequence having at least 50%, 60%, 70%, 75%, 80%, 85%, 90%, 91%, 92%, 93%, 94%, 95%, 96%, 97%, 98%, or 99% identity, preferably over the whole length, to the the nucleotide sequence of (i), (ii) or (iii), (v) a nucleotide sequence hybridzing the nucleotide sequence of (iii) under stringent conditions, (vi) a nucleotide sequence encoding a protein comprising the amino acid sequence set forth in any one of SEQ ID NOs: 238 to 258, (vii) a nucleotide sequence encoding a protein comprising the amino acid sequence at least 50%, 60%, 70%, 75%, 80%, 85%, 90%, 91%, 92%, 93%, 94%, 95%, 96%, 97%, 98%, or 99% identical to a sequence set forth in any one of SEQ ID NOs: 238 to 258, or (viii) a nucleotide sequence encoding a homologue, analogue or orthologue of protein comprising the amino acid sequence set forth in any one of SEQ ID NOs: 238 to 258.

In still another embodiment of the methods for modifying the genetic material of a cellular system at a predetermined location of the present invention, the synthetic transcription factor may be configured to modulate expression, preferably transcription, of the morphogenic gene by binding to a regulation region located at a certain distance in relation to the start codon.

In one embodiment of the methods for modifying the genetic material of a cellular system at a predetermined location of the present invention, the synthetic transcription factor and/or the at least one recognition domain comprises a sequence set forth in any one of SEQ ID NOs: 1 to 94, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 1 to 94, or wherein the synthetic transcription factor and/or at least one recognition domain, binds to a regulation region set forth in SEQ ID NOs: 95 to 190, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 95 to 190.

In one embodiment of the methods for modifying the genetic material of a cellular system at a predetermined location of the present invention, the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

In another embodiment of the methods for modifying the genetic material of a cellular system at a predetermined location of the present invention, the cellular system may be selected from the group consisting of at least one eukaryotic cell or eukaryotic organism, preferably wherein the at least one eukaryotic cell may be at least one plant cell, and/or wherein the at least one eukaryotic organism is a plant or a part of a plant.

In one embodiment, the at least one part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In another embodiment, the at least one plant cell, the at least one plant or the at least one part of a plant originates from a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicerjudaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In yet another embodiment of the methods for modifying the genetic material of a cellular system at a predetermined location of the present invention, the one or more nucleotide sequence(s) flanking the at least one nucleotide sequence of interest at the predetermined location may be at least 85%-100% complementary to the one or more nucleotide sequence(s) adjacent to the predetermined location, upstream and/or downstream from the predetermined location, over the entire length of the respective adjacent region(s).

In one embodiment of the methods for modifying the genetic material of a cellular system at a predetermined location of the present invention, the at least one nucleotide sequence of interest may be selected from the group consisting of: a transgene, a modified endogenous gene, a synthetic sequence, an intronic sequence, a coding sequence or a regulatory sequence. If the at least one nucleotide sequence of interest is a transgene, the transgene may comprise a nucleotide sequence encoding a gene of a genome of an organism of interest, or at least a part of said gene.

In another embodiment of the methods for modifying the genetic material of a cellular system at a predetermined location of the present invention, the at least one nucleotide sequence of interest may be a transgene of an organism of interest, wherein the transgene or part of the transgene may selected from the group consisting of a gene encoding resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, resistance or tolerance to 2,4-D, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content.

In yet another embodiment of the methods for modifying the genetic material of a cellular system at a predetermined location of the present invention, the at least one nucleotide sequence of interest may be at least part of a modified endogenous gene of an organism of interest, wherein the modified endogenous gene may comprise at least one deletion, insertion and/or substitution of at least one nucleotide in comparison to the nucleotide sequence of the unmodified endogenous gene, and/or the at least one nucleotide sequence of interest may be at least part of a modified endogenous gene of an organism of interest, wherein the modified endogenous gene may comprise at least one of a truncation, duplication, substitution and/or deletion of at least one nucleotide position encoding a domain of the modified endogenous gene.

In still another embodiment of the methods for modifying the genetic material of a cellular system at a predetermined location of the present invention, the at least one nucleotide sequence of interest may be at least part of a regulatory sequence, wherein the regulatory sequence may comprise at least one of a core promoter sequence, a proximal promoter sequence, a cis regulatory sequence, a trans regulatory sequence, a locus control sequence, an insulator sequence, a silencer sequence, an enhancer sequence, a terminator sequence, and/or any combination thereof.

Further provided is an embodiment of the methods according to the various aspects disclosed herein, wherein the at least one site-specific nuclease or a catalytically active fragment thereof, may be introduced into the cellular system as a nucleic acid sequence encoding the site-specific nuclease or the catalytically active fragment thereof, wherein the nucleic acid sequence is part of at least one vector, or wherein the at least one site-specific nuclease or the catalytically active fragment thereof, is introduced into the cellular system as at least one amino acid sequence. In one embodiment, the at least one site-specific nuclease may be introduced as translatable RNA. In yet a further embodiment, the at least one site-specific nuclease may be introduced as part of a complex together with at least one further biomolecule, for example, a gRNA, the gRNA optionally being associated with a RT comprising or being associated with the at least one nucleic acid sequence of interest to be introduced into the cellular system.

In another aspect of the present invention, there is provided a method of selecting an optimum synthetic transcription factor (STF) for modulating, preferably activating, the expression of at least one gene of interest, preferably a morphogenic gene, wherein the method comprises (i) defining a gene of interest; (ii) defining and providing at least one recognition domain, wherein the recognition domain is designed to recognize a recognition site at or near the gene of interest; (iii) defining and providing at least one activation domain; (iv) optionally: providing at least one further element, the element being selected from at least one promoter, at least one NLS, at least one transactivation domain, and/or at least one tag; (iv) providing at least two STFs targeting the same gene of interest; (v) measuring the modulation rate of each individual STF tested; (vi) selecting the STF with the best modulation rate for a given gene of interest. Furthermore, the method described herein, may also be used to select at least two optimum STFs for modulating to finetune transcription of at least two morphogenic gene of interest and to increase transformation and regeneration.

According to the various embodiments provided herein and due to the modular nature of the STFs, more than one STF can be designed for modulating a given gene of interest. Due to sterical issues and potential off-target effects in complex eukaryotic genomes it might thus be favorable to provide different STFs comprising a different number of domains and a different domain architecture, e.g., by domain shuffling, or by testing a TALE-based versus a CRISPR-based STF, to ultimately select the best STF for a target gene of choice.

In another aspect of the present invention, there is provided a method of producing a haploid or double haploid organism or cellular system, wherein the method may comprise the following steps: (a) providing a haploid cellular system; (b) introducing into the haploid cellular system at least one synthetic transcription factor, or a nucleotide sequence encoding the same; (c) culturing the haploid cellular system under conditions to obtain at least one haploid or double haploid organism; and (d) optionally: selecting the at least one haploid or double haploid organism obtained in step (c), wherein the at least one synthetic transcription factor, or the nucleotide sequence encoding the same, may comprise at least one recognition domain and at least one activation domain, wherein the at least one synthetic transcription factor may be configured to modulate the expression, preferably the transcription, of at least one morphogenic gene in the haploid cellular system.

As haploids are homozygous at all loci and can represent a new variety (self-pollinated crops) or parental inbred line for the production of hybrid varieties (cross-pollinated crops) which makes them attractive cell types in plant breeding programs. Still, haploids are usually smaller and exhibit lower plant vigor compared to wild-type donor plants and are sterile due to the inability of their chromosomes to pair during meiosis. Therefore, the synthetic transcription factors and methods provided herein can be used in the development of haploid cells, cellular systems and plants, as the introduction of at least one synthetic transcription factor, or a nucleotide sequence encoding the same of the present invention into a haploid cellular system can dramatically increase the reproductive capabilities of the haploid cellular system to develop into a haploid embryo, which in turn can be used as basis for haploid and double haploid plants.

A “double haploid” cell, cellular system or organism is obtained through spontaneous chromosome doubling during the step of culturing a haploid cell or cellular system, or through induced chromosome doubling after selecting the obtained haploid organism. The terms “double haploid” and “doubled haploid” are used interchangeably herein.

In one embodiment, in the method of producing a haploid or double haploid organism, the haploid cellular system of step (a) is a haploid embryo, or wherein the at least one haploid or double haploid organism defined in step (c) is obtained through an intermediate step of generating at least one haploid embryo from the haploid cellular system of (b).

Many plant cells have the ability to regenerate a complete organism from only single cells or tissues. This process is usually referred to as totipotency. A wide variety of cells have the potential to develop into embryos, including haploid gametophytic cells, such as the cells of pollen and embryo sacs (see Forster, B. P., et al. (2007) Trends Plant Sci. 12: 368-375 and Segui-Simarro, J. M. (2010) Bot. Rev. 76: 377-404), as well as somatic cells derived from all three tissue layers of the plant (Gaj, M. D. (2004) Plant Growth Regul. 43: 27-47 or Rose, R., et al. (2010) “Developmental biology of somatic embryogenesis” in: Plant Developmental Biology-Biotechnological Perspectives, Pua E-C and Davey M R, Eds. (Berlin Heidelberg: Springer), pp. 3-26). Embryo development also occurs in the absence of egg cell fertilisation during apomixis, a type of asexual seed development. Totipotency in apomictic plants is restricted to the gametophytic and sporophytic cells that normally contribute to the development of the seed and its precursors, including the unfertilised egg cell and surrounding sporophytic tissues (see Bicknell, R. A., and Koltunow, A. M. (2004) Plant Cell 16: S228-S245).

Notably, the phenomenon of totipotency of plant cells reaches its highest expression in tissue culture, i.e., in vitro. Therefore, relevant steps for haploid generation start from immature cell cultures in vitro which have to be treated under suitable conditions to induce embryogenesis. These steps usually are time-consuming and often rather inefficient, as only a small minority of cultured haploid cellular systems will mature to a morphological and cellular state, optionally comprising any further GE event, in a desired way. Assisted by the synthetic transcription factors and the methods disclosed herein, the generation of haploid and/or doubled haploid systems can thus be significantly enhanced, as the methods provide a cellular system having a much higher regenerative capability guaranteeing a higher frequency of positive events.

In one embodiment of the methods of producing a haploid or double haploid cellular system or organism, the methods may comprise an additional step of inducing microspore-derived embryogenesis. Microspore-derived embryogenesis is a unique process in which haploid, immature pollen (microspores) are induced by one or more stress treatments to form embryos in culture. These microspore-derived embryos can then be germinated and converted to homozygous doubled haploid plants by chromosome doubling agents and/or through spontaneous doubling. Double haploid production, as detailed above, is a major tool in plant breeding and trait discovery programs as it allows homozygous lines to be produced in a single generation. This quick route to homozygosity not only drastically reduces the breeding period, but also unmasks traits controlled by recessive alleles. Doubled haploids are widely used in crop improvement as parents for F1 hybrid seed production, to facilitate backcross conversion, for mutation breeding, and to generate immortal populations for molecular mapping studies.

The term “immature” as used herein in the context of a cellular system is intended to mean any immature cell or genetic material obtainable from a plant. “Immature” cells or cellular systems may include male or female immature cells, or immature vegetative cells. Immature female or male cells or cellular systems may be selected from immature embryos or immature callus tissue, male gametophyte, e.g., microspore, or vegetative, generative or sperm cells of the pollen grain, or female gametophytes, including a megaspore and its derivatives, including the egg cell, the polar nuclei, the central cell, the synergids, the antipodals. The female gametophyte material may be comprised in an ovule and the ovule may represent a cellular system according to the present invention. Where a microspsore is used as haploid cellular system of the present invention, a callus may be formed which may then undergo organogenesis to form an embryo.

Methods for obtaining haploid and double haploid cellular systems and organisms using chemical approaches are known to the skilled person (see, for example, WO 2015/044199 A1). According to certain embodiments of the methods for producing a haploid cellular system, the methods may thus comprise an additional step of treating or culturing a haploid cellular system prior to introducing into the haploid cellular system at least one synthetic transcription factor, or a nucleotide sequence encoding the same of the present invention, wherein the additional step of treating or culturing may comprise adding a histone deacetylase inhibitor or at least one chemical to the developing cellular system. A histone deacetylase inhibitor (HDACi) is preferably a compound which is capable of interacting with a histone deacetylase and inhibiting its enzymatic activity, thereby reducing the ability of a histone deacetylase to remove an acetyl group from a histone and may include, for example, hydroxamic acids (other than salicyl hydroxamic acid), cyclic tetrapeptides, aliphatic acids, benzamides, polyphenols or electrophilic ketones, trichostatin A (TSA), butyric acid, a butyrate salt, potassium butyrate, sodium butyrate, ammonium butyrate, lithium butyrate, phenylbutyrate, sodium phenylbutyrate or sodium n-butyrate, wherein the term butyric acid in the context of this specification does not include isobutyric acid or α,β-dichlorobutyric acid, or suberoylanilide hydroxamic acid all compounds being commercially available.

In another embodiment, physical stress may be applied to the haploid cellular system or organism. The physical stress may be any of temperature, darkness, light or ionizing radiation, for example. The light may be full spectrum sunlight, or one or more frequencies selected from the visible, infrared or UV spectrum. One or more physical stresses or combinations of stress may be used. The stresses may be continuous or interrupted (periodic); regular or random over time. When stresses are combined over time they may be simultaneous (coterminous or partly overlapping) or separate.

In a further embodiment, an additional step of adding chemical stress may be applied in the methods of the present invention. Haploid embryo development or microspore embryogenesis, pollen embryogenesis or androgenesis, can thus be additionally induced by exposing anthers or isolated gametophytes to abiotic or chemical stress during in vitro culture (Touraev, A., et al (1997) Trends Plant Sci. 2: 297-302).

In a further embodiment the method of producing a haploid cellular system or organism may comprise an additional step of generating at least one doubled haploid cellular system or organism from the haploid cellular system.

In yet a further embodiment the method of producing a haploid or double haploid cellular system or organism may comprises an additional step of generating seedling from the at least one haploid cellular system or organism, or from the at least one doubled haploid cellular system or organism. The ability of haploid embryos to convert spontaneously or after treatment with chromosome doubling agents to double-haploid plants is widely exploited and known to the skilled person (Touraev, A., et al. (1997) Trends Plant Sci. 2: 297-302; Forster et al. (2007) supra). In certain embodiments, haploid embryogenesis and chromosome doubling may take place substantially simultaneously. In other embodiments, there may be a time delay between haploid embryogenesis and chromosome doubling. The time delay may relate to the developmental stage reached by the growing haploid embryo, seedling or plantlet. Should growth of haploid seedlings, plants or plantlets not involve a spontaneous chromosome doubling event, then a chemical chromosome doubling agent may be used in accordance with procedures which the average skilled person will be familiar with. Chromosome doubling and chromosome doubling agents suitable according to the various aspects and embodiments of the present invention are provided in Segui-Simarro J. M., & Nuez F. (2008) Cytogenet. Genome Res. 120: 358-369). Suitable chromosome doubling agents include, for example, colchicine, anti-microtubule agents or anti-microtubule herbicides such as pronamide, nitrous oxide, or any mitotic inhibitor. Where colchicine is used, the concentration in the medium may be generally 0.01%-0.2% or approximately 0.05% or APM (5-225 μM). The range of colchicine concentration may be from about 400-600 mg/L or about 500 mg/L. Where pronamide is used the medium concentration may be about 0.5-20 μM. Other agents such as DMSO, adjuvants or surfactants may be used with the mitotic inhibitors to improve doubling efficiency. Common or trade names of suitable chromosome doubling agents include: colchicine, acetyltrimethylcolchicinic acid derivatives, carbetamide, chloropropham, propham, pronamide/propyzamide tebutam, chlorthal dimethyl (DCPA), Dicamba/dianat/disugran (dicamba-methyl) (BANVEL, CLARITY), benfluralin/benefin/(BALAN), butralin, chloralin, dinitramine, ethalfluralin (Sonalan), fluchloralin, isopropalin, methalpropalin, nitralin, oryzalin (SURFLAN), pendimethalin, (PROWL), prodiamine, profluralin, trifluralin (TREFLAN, TRIFIC, TRILLIN), AMP (Amiprofos methyl); amiprophos-methyl Butamifos, Dithiopyr and Thiazopyr. The result of applying said agents is a homozygous double haploid cell or cellular system, organism.

In one embodiment of the above methods, the at least one synthetic transcription factor, or a sequence encoding the same, or at least one component of the at least one synthetic transcription factor, or the sequence encoding the same, may be introduced into the haploid cellular system by means independently selected from biological and/or physical means, including transfection, transformation, including transformation by Agrobacterium spp. transformation, preferably by Agrobacterium tumefaciens, a viral vector, biolistic bombardment, transfection using chemical agents, including polyethylene glycol transfection, or any combination thereof.

In another embodiment of the above methods, the at least one recognition domain is or is a fragment of at least one disarmed CRISPR/nuclease system.

In one embodiment of the above methods, the at least one disarmed CRISPR/nuclease system is a CRISPR/dCpf1 system, wherein the at least one disarmed CRISPR/nuclease system comprises at least one guide RNA.

In a further preferred embodiment of the various aspects of the present invention, the recognition domain of the STF comprises a disarmed LbCpf1 domain (SEQ ID NO: 282) a disarmed LbCpf1_RR domain (SEQ ID NO: 283) and/or a disarmed LbCpf1_RVR domain (SEQ ID NO: 284). To increase the efficiency of transcriptional regulation, preferably activation, gRNAs of the CRISPR/Cpf1 system are preferred which target a region up to 250 bp upstream of the transcription start site. In one embodiment of the herein described aspects of the invention, preferred gRNAs target a region within a range of 1-250, 1-200, 1-150, 1-100, 1-50, 50-250, 100-250, 150-250 or 200-250 bp upstream of the transcription start site, or any range in between the herein disclosed ranges.

In preferred embodiment, the method of providing a haploid or double haploid cellular system or organism may utilize at least one synthetic transcription factor comprising at least one recognition and at least one activation domain as further disclosed herein above, wherein said embodiments and aspects relating to a synthetic transcription factor of the present invention may be employed to provide optimized methods for obtaining a haploid or a doubled haploid cellular system or organism.

In a further embodiment of the method of providing a haploid or double haploid cellular system or organism, the at least one activation domain of the at least one synthetic transcription factor is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof. In a preferred embodiment of the invention the at least one activation domain is VPR (SEQ ID NO: 276). In a further preferred embodiment of the present invention, a combination of different activation domains can be used, e.g. VP64-p65-Rita or any combination of activation domains commonly known in the art.

Suitable linkers for the herein described CRISPR/Cpf1 systems comprise flexible linkers, such as 5GS or XTEN, while in vivo cleavable linkers are not suitable for the herein described aspects of the invention.

In another embodiment of the method of providing a haploid or double haploid cellular system or organism, the at least one activation domain of the at least one synthetic transcription factor is located N-terminal and/or C-terminal relative to the at least one recognition domain of the at least one synthetic transcription factor.

In a preferred embodiment of the method of providing a haploid or double haploid cellular system or organism of the present invention, the recognition domain of the STF is or is a fragment of at least one disarmed CRISPR/Cpf1 system and the activation domain is a VPR domain, optionally with a linker inbetween the recognition domain and the activation domain, preferably a 5×GS linker.

Preferred morphogenic genes to be modified according to the methods disclosed herein may be selected from the group consisting of BBM, WUS, a WOX gene, a WUS or BBM homologue, Lec1, Lec2, WIND1, ESR1, PLT3, PLT5, PLT7, IPT, IPT2, Knotted1, and RKD4. More preferred morphogenic genes to be modified according to the methods disclosed herein may be agene comprising a nucleotide sequence selected from the group consisting of (i) a nucleotide sequence set forth in any one of SEQ ID NOs: 199 to 237, (ii) a nucleotide sequence having the coding sequences of the nucleotide sequence set forth in any one of SEQ ID NOs: 199 to 237, (iii) a nucleotide sequence complementary to the nucleotide sequence of (i) or (ii), (iv) a nucleotide sequence having at least 50%, 60%, 70%, 75%, 80%, 85%, 90%, 91%, 92%, 93%, 94%, 95%, 96%, 97%, 98%, or 99% identity, preferably over the whole length, to the the nucleotide sequence of (i), (ii) or (iii), (v) a nucleotide sequence hybridzing the nucleotide sequence of (iii) under stringent conditions, (vi) a nucleotide sequence encoding a protein comprising the amino acid sequence set forth in any one of SEQ ID NOs: 238 to 258, (vii) a nucleotide sequence encoding a protein comprising the amino acid sequence at least 50%, 60%, 70%, 75%, 80%, 85%, 90%, 91%, 92%, 93%, 94%, 95%, 96%, 97%, 98%, or 99% identical to a sequence set forth in any one of SEQ ID NOs: 238 to 258, or (viii) a nucleotide sequence encoding a homologue, analogue or orthologue of protein comprising the amino acid sequence set forth in any one of SEQ ID NOs: 238 to 258.

In one embodiment of the method of providing a haploid or double haploid cellular system or organism, the synthetic transcription factor is configured to modulate expression, preferably transcription, of the morphogenic gene by binding to a regulation region located at a certain distance in relation to the start codon.

In another embodiment of the method of providing a haploid or double haploid cellular system or organism, the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

In one embodiment, the at least one haploid cellular system may be selected from the group consisting of at least one eukaryotic cell or eukaryotic organism, preferably wherein the at least one eukaryotic cell may be at least one plant cell, and/or wherein the at least one eukaryotic organism may be a plant or a part of a plant.

In a further embodiment, the at least one part of the plant may be selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, pericycles, and seeds.

In a further embodiment, the plant cell, the at least one plant or part of a plant originates from a plant species which may be selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In one aspect, the present invention relates to a cellular system or a progeny thereof, which is obtained by a method for increasing the transformation efficiency in a cellular system according to any of the embodiments described above.

In another aspect, the present invention relates to a cellular system or a progeny thereof, which is obtained by a method of modifying the genetic material of a cellular system at a predetermined location according to any of the embodiments described above.

In a further aspect, the present invention relates to a haploid or double haploid organism, which is obtained by a method of producing a haploid or double haploid organism according to any of the embodiments above.

In one aspect of the present invention, at least one cellular system, at least one haploid cellular system and/or at least one haploid or double(d) haploid cellular system or organism may be provided obtainable by the methods disclosed herein using at least one synthetic transcription factor specifically modulating the transcription of at least one morphogenic gene of interest. The cellular system such obtained may then be used for further genome editing methods as used herein, or for regenerating a plant from the modified cellular system.

In one aspect of the present invention, there is provided a method or use based on a synthetic transcription factor, or a sequence encoding the same, according to the various methods as disclosed herein.

In one aspect, the invention also provides a use of a synthetic transcription factor according to any of the embodiments described above, or a sequence encoding the same, in a method for increasing the transformation efficiency in a cellular system according to any of the embodiments described above.

In another aspect, the invention also provides a use of a synthetic transcription factor according to any of the embodiments described above, or a sequence encoding the same, in a method of modifying the genetic material of a cellular system at a predetermined location according to any of the embodiments described above.

In a further aspect, the invention also provides a use of a synthetic transcription factor according to any of the embodiments described above, or a sequence encoding the same, in a method of producing a haploid or double haploid organism according to any of the embodiments described above.

By using the synthetic transcription factor of the present invention, it is possible to activate the expression of endogenous genes in a cellular system. Multiple endogenous genes can specifically be targeted for enhanced expression in a transient manner and in a transgene-free environment. The means and methods described herein, therefore have a wide range of possible applications.

In one aspect, there is provided a synthetic transcription factor, or a nucleotide sequence encoding the same, comprising at least one recognition domain and at least one activation domain, wherein the synthetic transcription factor is configured to activate the expression of an endogenous gene in a cellular system.

In a preferred embodiment, the at least one recognition domain is, or is a fragment of at least one disarmed CRISPR/nuclease system.

In a further preferred embodiment, the at least one disarmed CRISPR/nuclease system is a CRISPR/dCpf1 system, wherein the at least one disarmed CRISPR/nuclease system comprises at least one guide RNA.

In a further preferred embodiment of the various aspects of the present invention, the recognition domain of the STF comprises a disarmed LbCpf1 domain (SEQ ID NO: 282) a disarmed LbCpf1_RR domain (SEQ ID NO: 283) and/or a disarmed LbCpf1_RVR domain (SEQ ID NO: 284). To increase the efficiency of transcriptional regulation, preferably activation, gRNAs of the CRISPR/Cpf1 system are preferred which target a region up to 250 bp upstream of the transcription start site. In one embodiment of the herein described aspects of the invention, preferred gRNAs target a region within a range of 1-250, 1-200, 1-150, 1-100, 1-50, 50-250, 100-250, 150-250 or 200-250 bp upstream of the transcription start site, or any range in between the herein disclosed ranges.

In one embodiment, the at least one activation domain is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof. In a preferred embodiment, the at least one activation domain is VPR (SEQ ID NO: 276). In a further preferred embodiment of the present invention, a combination of different activation domains can be used, e.g. VP64-p65-Rita or any combination of activation domains commonly known in the art.

Suitable linkers for the herein described CRISPR/Cpf1 systems comprise flexible linkers, such as 5GS or XTEN, while in vivo cleavable linkers are not suitable for the herein described aspects of the invention. In another embodiment, the at least one activation domain is located N-terminal and/or C-terminal relative to the at least one recognition domain.

In a preferred embodiment of the synthetic transcription factor of the present invention, the recognition domain of the STF is or is a fragment of at least one disarmed CRISPR/Cpf1 system and the activation domain is a VPR domain, optionally with a linker inbetween the recognition domain and the activation domain, preferably a 5×GS linker.

In a further embodiment, the endogenous gene is selected from the group consisting of a gene encoding a monogenic or polygenic crop trait, preferably a gene encoding resistance or tolerance to abiotic stress, including drought stress, osmotic stress, heat stress, cold stress, oxidative stress, heavy metal stress, nitrogen deficiency, phosphate deficiency, salt stress or waterlog-ging, herbicide resistance, including resistance to glyphosate, glufosinate/phosphinotricin, hygromycin, resistance or tolerance to 2,4-D, proto-porphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content. Specific preferred examples are ZmZEP1 (SEQ ID NO 309), ZmRCA-beta (SEQ ID NO 310), BvEPSPS (SEQ ID NO 311), and BvFT2 (SEQ ID NO 312).

Further preferred embodiments of the present invention include increased expression of the Na+/H+ antiporter to induce salt tolerance in tomato plants (Zhang H X and Blumwald E (2001), Transgenic salt-tolerant tomato plants accumulate salt in foliage but not in fruit, Nature Biotechnpology 19, 765-768), BvTST2.1 overexpression to increase sucrose yield in taproots (Jung et al. (2015), Identification of the transporter responsible for sucrose accumulation in sugar beet taproots, Nature Plants 1, 14001), overexpression of small and large subunits from Rubisco with the Rubisco assembly chaperone RUBISCO ASSEMBLY FACTOR 1 (RAF1) for improving corn productivity (Salesse-Smith C E et al. (2018), Overexpression of Rubisco subunits with RAF1 increases Rubisco content in maize, Nature Plants 2, 802-810), overexpression of ZmArgos to increase drought tolerance (Shi J et al. (2015), Overexpression of ARGOS genes modifies plant sensitivity to ethylene, leading to improved drought tolerance in both Arabidopsis and maize, Plant Physiology 169(1), 266-282), and activation of HPPD gene expression to induce herbicide resistance (Nakka S et al. (2017), Physiological and molecular characterization of hydroxyphenylpyruvate diogygenase (HPPD)-inhibitor resistance in Palmer Amaranth (Amaranthus palmeri S.Wats), Frontiers in Plant Science 8, 555).

In one embodiment, the synthetic transcription factor is configured to activate expression, preferably transcription, of the endogenous gene by binding to a regulation region located at a certain distance in relation to the start codon.

In another embodiment, the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

In one embodiment, the cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

In another embodiment, the at least one part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In a further embodiment, the at least one plant cell, the at least one plant or the at least one part of a plant originates from a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulaturn, Cicerjudaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In another aspect, there is provided a method for increasing the expression of at least one endogenous gene in a cellular system, wherein the method comprises the steps of:
(a) providing a cellular system;
(b) introducing into the cellular system at least one synthetic transcription factor, or a nucleotide sequence encoding the same;

wherein the at least one synthetic transcription factor, or the nucleotide sequence encoding the same, comprises at least one recognition domain and at least one activation domain,

wherein the synthetic transcription factor is configured to increase the expression, preferably the transcription, of at least one endogenous gene in the cellular system.

In a preferred embodiment, the at least one recognition domain is, or is a fragment of at least one disarmed CRISPR/nuclease system.

In a further preferred embodiment, the at least one disarmed CRISPR/nuclease system is a CRISPR/dCpf1 system, wherein the at least one disarmed CRISPR/nuclease system comprises at least one guide RNA.

In a further preferred embodiment of the various aspects of the present invention, the recognition domain of the STF comprises a disarmed LbCpf1 domain (SEQ ID NO: 282) a disarmed LbCpf1_RR domain (SEQ ID NO: 283) and/or a disarmed LbCpf1_RVR domain (SEQ ID NO: 284). To increase the efficiency of transcriptional regulation, preferably activation, gRNAs of the CRISPR/Cpf1 system are preferred which target a region up to 250 bp upstream of the transcription start site. In one embodiment of the herein described aspects of the invention, preferred gRNAs target a region within a range of 1-250, 1-200, 1-150, 1-100, 1-50, 50-250, 100-250, 150-250 or 200-250 bp upstream of the transcription start site, or any range in between the herein disclosed ranges.

In one embodiment, the at least one activation domain is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof. In a preferred embodiment, the at least one activation domain is VPR (SEQ ID NO: 276). In a preferred embodiment, the at least one activation domain is VPR (SEQ ID NO: 276). In a further preferred embodiment of the present invention, a combination of different activation domains can be used, e.g. VP64-p65-Rita or any combination of activation domains commonly known in the art.

Suitable linkers for the herein described CRISPR/Cpf1 systems comprise flexible linkers, such as 5GS or XTEN, while in vivo cleavable linkers are not suitable for the herein described aspects of the invention. In another embodiment, the at least one activation domain is located N-terminal and/or C-terminal relative to the at least one recognition domain.

In a preferred embodiment of the method for increasing the expression of at least one endogenous gene in a cellular system of the present invention, the recognition domain of the STF is or is a fragment of at least one disarmed CRISPR/Cpf1 system and the activation domain is a VPR domain, optionally with a linker inbetween the recognition domain and the activation domain, preferably a 5×GS linker.

In a further embodiment, the endogenous gene is selected from the group consisting of a gene encoding resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, resistance or tolerance to 2,4-D, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content.

In one embodiment, the synthetic transcription factor is configured to activate expression, preferably transcription, of the endogenous gene by binding to a regulation region located at a certain distance in relation to the start codon.

In another embodiment, the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

In one embodiment, the cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

In another embodiment, the at least one part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In a further embodiment, the at least one plant cell, the at least one plant or the at least one part of a plant originates from a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicerjudaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

Due to the modular character of the synthetic transcription factors disclosed herein, there may also be provided at least one synthetic transcription factor comprising at least one recognition domain as disclosed herein and further comprising a silencing domain. The silencing domain thus substitutes the activation domain to provide a highly specific synthetic transcription factor for modulating, in this setting decreasing, the transcription of a gene of interest.

Transcriptional repression in eukaryotes is achieved through “silencers”, of which there are different types, namely “silencer elements” and “negative regulatory elements” (NREs). Silencer elements are classical, position-independent elements that direct an active repression mechanism, and NREs are position-dependent elements that direct a passive repression mechanism. In addition, “repressors” are DNA-binding transcription factors that interact directly with silencers. The silencer itself and its context within a given promoter, rather than the interacting repressor, usually determines the mechanism of repression. Silencers form an intrinsic part of many eukaryotic promoters and are thus highly important for gene regulation in eukaryotes, including plant and animal cells. Silencer elements can be located in the 5′ or 3′ direction relative to a transcription initiation site.

Therefore, the synthetic transcription factors of the present invention, or a nucleotide sequence encoding the same, can also comprise at least one recognition domain and at least one silencing domain, wherein the synthetic transcription factor is configured to modulate the expression of a morphogenic gene in a cell or cellular system of interest, preferably in a plant cell.

In one aspect there is provided a method for producing a transgenic cellular system or organism comprising performing any of the method as detailed herein, wherein the method further comprises the regeneration of a cellular system or organism comprising at least one nucleotide sequence of interest as a transgene. A “transgene” in this context refers to any nucleic acid sequence artificially introduced into a cell, cellular system or organism.

According to certain embodiments, the method for producing a transgenic cellular system or organism may preferably use the synthetic transcription factors as disclosed herein to obtain a higher transformation frequency and/or regeneration rate of the such transformed material.

In yet another aspect there is provided a method for producing a genetically modified cellular system or organism, wherein the method may comprise performing a method of modifying the genetic material of a cellular system at a predetermined location detailed herein above, wherein the method further comprises the regeneration of a cellular system or organism comprising a modification at a predetermined location in the genetic material of the cellular system or organism. Again, said methods rely on the use of a synthetic transcription factor according to the various aspects and embodiments of the present invention. This aspect can be advantageously used for the transient introduction of at least one construct or genetic material into a cell or cellular system of interest to modify the transcription of a gene of interest, preferably a morphogenic gene, in a targeted way to boost the regenerability of the targeted cell or cellular system potentially harboring the insertion and/or deletion and/or edit. This, in turn, dramatically decreases the number of cells to be screened for a positive genetic modification or edit.

In one embodiment according to the various aspects of the present invention, the at least one nucleic acid sequence of interest may be provided as part of at least one vector, or as at least one linear molecule. In another aspect, the at least one nucleic acid sequence of interest may be provided as a complex, preferably a complex physically associating the at least one nucleic acid sequence and another RT, and/or with a gRNA, and/or with a site-specific nuclease. The at least one nucleic acid sequence of interest may further comprise a sequence allowing the rapid traceability, including the visual traceability, of the sequence of interest, e.g., a tag, including a fluorescent tag. The at least one nucleic acid sequence of interest may be double-stranded, single-stranded, or a mixture thereof. Furthermore, the at least one nucleic acid sequence of interest may comprise a mixture of DNA and RNA nucleotide, including also synthetic, i.e., non-naturally occurring nucleotides.

Delivery and analytical methods:

Any suitable delivery method to introduce at least one biomolecule into a cell or cellular system can be applied, depending on the cell or cellular system of interest. The term “introduction” as used herein thus implies a functional transport of a biomolecule or genetic construct (DNA, RNA, single- or double-stranded, protein, comprising natural and/or synthetic components, or a mixture thereof) into at least one cell or cellular system, which allows the transcription and/or translation and/or the catalytic activity and/or binding activity, including the binding of a nucleic acid molecule to another nucleic acid molecule, including DNA or RNA, or the binding of a protein to a target structure within the at least one cell or cellular system, and/or the catalytic activity of an enzyme such introduced, optionally after transcription and/or translation. Where pertinent, a functional integration of a genetic construct may take place in a certain cellular compartment of the at least one cell, including the nucleus, the cytosol, the mitochondrium, the chloroplast, the vacuole, the membrane, the cell wall and the like. Consequently, the term “functional integration” implies that a molecular complex of interest is introduced into the at least one cell or cellular system by any means of transformation, transfection or transduction by biological means, including Agrobacterium transformation, or physical means, including particle bombardment, as well as the subsequent step, wherein the molecular complex can exert its effect within or onto the at least one cell or cellular in which it was introduced regardless of whether the construct or complex is introduced in a stable or in a transient way.

According to the various embodiments, at least one STF according to the present invention may thus be provided in the form of at least one vector, e.g., a plasmid vector, as at least one linear molecule, or as at least one complex pre-assembled ex vivo.

Depending on the nature of the genetic construct or biomolecule to be introduced, said effect naturally can vary and including, alone or in combination, inter alia, the transcription of a DNA encoded by the genetic construct to a ribonucleic acid, the translation of an RNA to an amino acid sequence, the activity of an RNA molecule within a cell, comprising the activity of a guide RNA, a crRNA, a tracrRNA, or an miRNA or an siRNA for use in RNA interference, and/or a binding activity, including the binding of a nucleic acid molecule to another nucleic acid molecule, including DNA or RNA, or the binding of a protein to a target structure within the at least one cell, or including the integration of a sequence delivered via a vector or a genetic construct, either transiently or in a stable way. Said effect can also comprise the catalytic activity of an amino acid sequence representing an enzyme or a catalytically active portion thereof within the at least one cell and the like. Said effect achieved after functional integration of the molecular complex according to the present disclosure can depend on the presence of regulatory sequences or localization sequences which are comprised by the genetic construct of interest as it is known to the person skilled in the art.

A variety of suitable transient and stable delivery techniques suitable according to the methods of the present invention for introducing genetic material, biomolecules, including any kind of single-stranded and double-stranded DNA and/or RNA, or amino acids, synthetic or chemical substances, into a eukaryotic cell, preferably a plant cell, or into a cellular system comprising genetic material of interest, are known to the skilled person, and comprise inter alia choosing direct delivery techniques ranging from polyethylene glycol (PEG) treatment of protoplasts (Potrykus et al. 1985), procedures like electroporation (D'Halluin et al., 1992), microinjection (Neuhaus et al., 1987), silicon carbide fiber whisker technology (Kaeppler et al., 1992), viral vector mediated approaches (Gelvin, Nature Biotechnology 23, “Viral-mediated plant transformation gets a boost”, 684-685 (2005)) and particle bombardment (see e.g. Sood et al., 2011, Biologic Plantarum, 55, 1-15). Transient transfection of mammalian cells with PEI is disclosed in Longo et al., Methods Enzymol., 2013, 529:227-240. Protocols for transformation of mammalian cells are disclosed in Methods in Molecular Biology, Nucleic Acids or Proteins, ed. John M. Walker, Springer Protocols.

For plant cells to be modified, despite transformation methods based on biological approaches, like Agrobacterium transformation or viral vector mediated plant transformation, and methods based on physical delivery methods, like particle bombardment or microinjection, have evolved as prominent techniques for introducing genetic material into a plant cell or tissue of interest. Helenius et al. (“Gene delivery into intact plants using the Helios™ Gene Gun”, Plant Molecular Biology Reporter, 2000, 18 (3):287-288) discloses a particle bombardment as physical method for introducing material into a plant cell.

Currently, there thus exists a variety of plant transformation methods to introduce genetic material in the form of a genetic construct into a plant cell or cellular system of interest, comprising biological and physical means known to the skilled person on the field of plant biotechnology which are applicable to the various introduction techniques of biomolecules or complexes thereof according to the present invention. Notably, said delivery methods for transformation and transfection can be applied to introduce the tools of the present invention simultaneously. A common biological means is transformation with Agrobacterium spp. which has been used for decades for a variety of different plant materials. Viral vector mediated plant transformation represents a further strategy for introducing genetic material into a cell of interest. Physical means finding application in plant biology are particle bombardment, also named biolistic transfection or microparticle-mediated gene transfer, which refers to a physical delivery method for transferring a coated microparticle or nanoparticle comprising a nucleic acid or a genetic construct of interest into a target cell or tissue. Physical introduction means are suitable to introduce nucleic acids, i.e., RNA and/or DNA, and proteins. Likewise, specific transformation or transfection methods exist for specifically introducing a nucleic acid or an amino acid construct of interest into a plant cell, including electroporation, microinjection, nanoparticles, and cell-penetrating peptides (CPPs). Furthermore, chemical-based transfection methods exist to introduce genetic constructs and/or nucleic acids and/or proteins, comprising inter alia transfection with calcium phosphate, transfection using liposomes, e.g., cationic liposomes, or transfection with cationic polymers, including DEAD-dextran or polyethylenimine, or combinations thereof. Said delivery methods and delivery vehicles or cargos thus inherently differ from delivery tools as used for other eukaryotic cells, including animal and mammalian cells and every delivery method may have to be specifically fine-tuned and optimized for a construct of interest for introducing and/or modifying the genetic material of at least one cellular system, plant cell, tissue, organ, or whole plant; and/or can be introduced into a specific compartment of a target cell of interest in a fully functional and active way.

The above delivery techniques, alone or in combination, can be used for in vivo (in planta) or in vitro approaches. According to the various embodiments of the present invention, different delivery techniques may be combined with each other, simultaneously or subsequently, for example, using a chemical transfection for the at least synthetic transcription factor, or the sequence encoding the same, one site-specific nuclease, or a mRNA or DNA encoding the same, and optionally further molecules, for example, a gRNA, whereas this is combined with the transient provision of the (partial) inactivation(s) using an Agrobacterium based technique.

A synthetic transcription factor of the present invention may thus be introduced together with, before, or subsequently to the transformation and/or transfection of relevant tools for inducing a targeted genomic edit and/or further chemicals to induce haploid or doubled haploid development.

Likewise, methods for analyzing a successful transformation or transfection event according to the present invention are known to the person skilled in the art and comprise, but are not limited to polymerase chain reaction (PCR), including inter alia real time quantitative PCR, multiplex PCR, RT-PCR, nested PCR, analytical PCR and the like, microscopy, including bright and dark field microscopy, dispersion staining, phase contrast, fluorescence, confocal, differential interference contrast, deconvolution, electron microscopy, UV microscopy, IR microscopy, scanning probe microscopy, the analysis of plant or plant cell metabolites, RNA analysis, proteome analysis, functional assays for determining a functional integration, e.g. of a marker gene or a transgene of interest, or of a knock-out, Southern-Blot analysis, sequencing, including next generation sequencing, including deep sequencing or multiplex sequencing and the like, and combinations thereof.

In yet another embodiment of the above aspect according to the present invention, the introduction of a construct of interest is conducted using physical and/or biological means selected from the group consisting of a device suitable for particle bombardment, including a gene gun, including a hand-held gene gun (e.g. Helios® Gene Gun System, BIO-RAD) or a stationary gene gun, transformation, including transformation using Agrobacterium spp. or using a viral vector, microinjection, electroporation, whisker technology, including silicon carbide whisker technology, and transfection, or a combination thereof.

The practice of the disclosed methods employs, unless otherwise indicated, conventional techniques in molecular biology, biochemistry, genetics, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989; Ausubel et al., CURRENT PROTOCOLS IN MOLECULAR BIOLOGY, John Wiley & Sons, New York, 1987 and periodic updates; and the series METHODS IN ENZYMOLOGY, Academic Press, San Diego.

The present invention is further described with reference to the following non-limiting examples.

### EXAMPLES

### Example 1: TAL Transcription Factors for Transient Expression of Endogenous Morphogenic Genes in Zea mays (Zm)

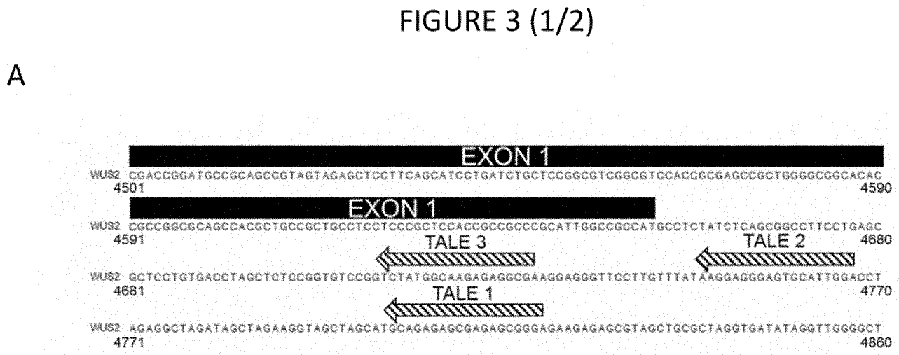

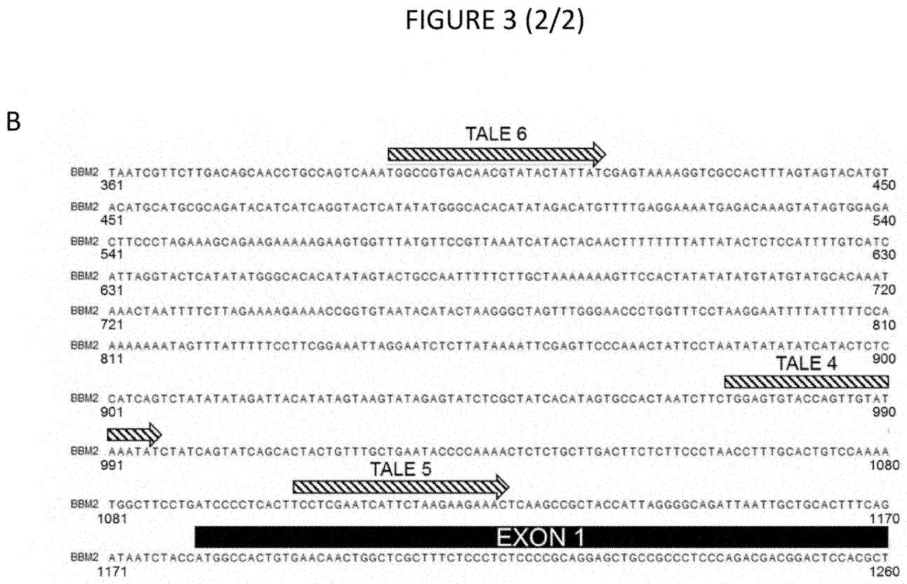

In one example, commercially designed and constructed TAL transcription factors are used to transiently increase the expression of BBM and WUS. The TAL transcription factors are designed to bind to about 24 bp of the regulation region of BBM set forth in SEQ ID NO: 95, 109 to 147 and 270 to 272 and/or about 18 bp of the regulation region of WUS set forth in SEQ ID NO: 96, 148 to 190 (see FIGS. 3 A and B). The TAL transcription factor recognition domains for BBM comprise a sequence set forth in SEQ ID NOs: 13 to 51 and/or the TAL transcription factor recognition domain for WUS comprise a sequence set forth in SEQ ID NO: 52 to 94.

The TAL Effector sequences can be designed and cloned, and an activation domain of Herpes simplex (VP16 or tetrameric VP64) can be added to the constructs in a fusion protein-like manner.

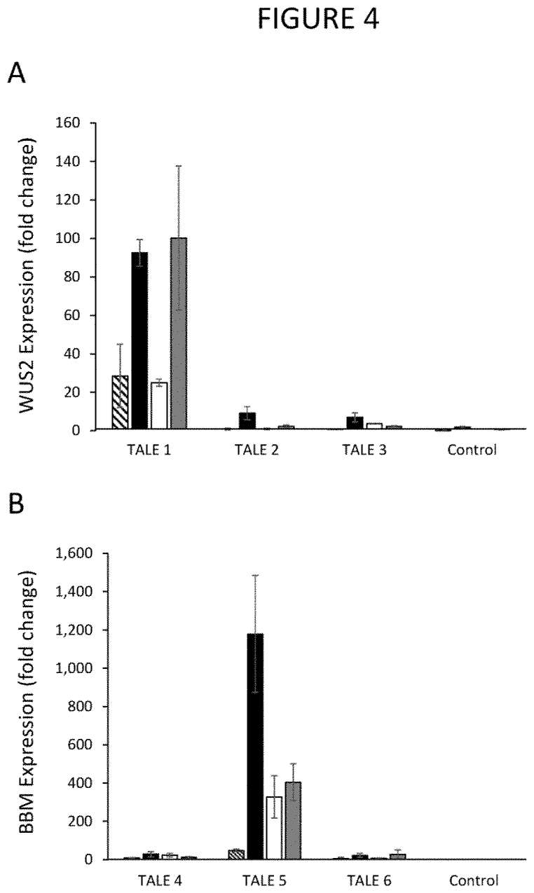

Transient induction of expression is first tested in maize protoplasts by PEG-mediated transformation and quantitative reverse transcriptase PCR or western blot against the ZmBBM and ZmWUS mRNA or protein respectively. To do this, 20 μg plasmid DNA encoding TALE transcription factors were delivered to approximately 600,000 protoplasts via a PEG-based transformation system commonly known in the art (see FIG. 4). The experiments were performed in triplicates and repeated four times (biological replicates). 24 hours after transformation, RNA was extracted and converted into cDNA using a commercially available kit. Expression of endogenous ZmWUS and ZmBBM was then determined using a SYBR Green qRT-PCR approach. The results clearly indicate that the synthetic transcription factors TALE1 (SEQ ID NO: 151) and TALE5 (SEQ ID NO: 271) are able to induce endogenous gene expression of WUS (60-fold induction) and BBM (490-fold induction), respectively (see FIGS. 4A and 4B).

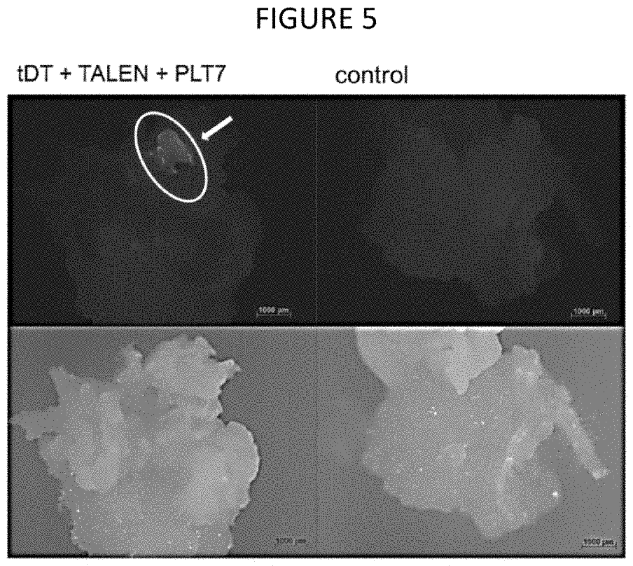

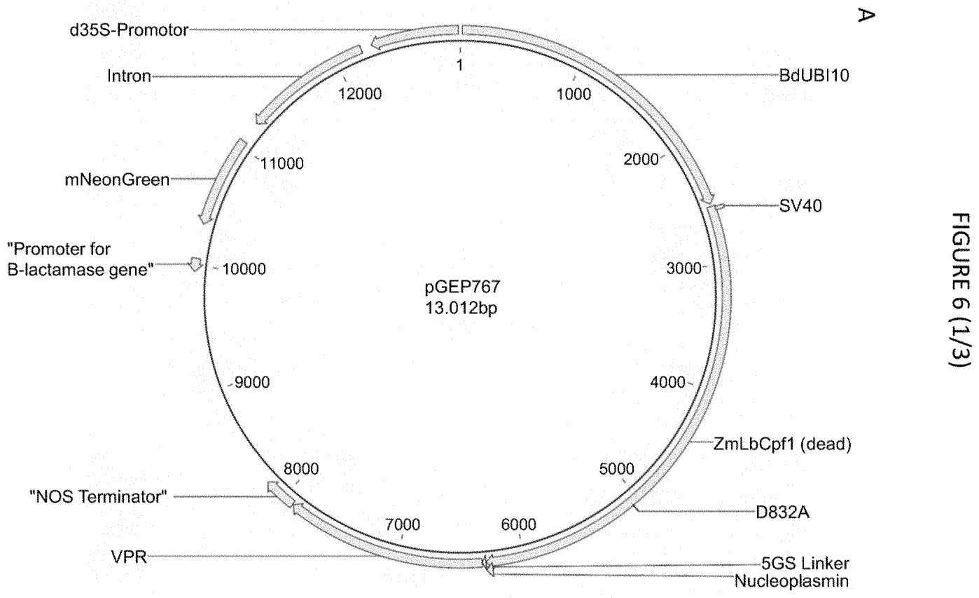

Next, the phenotypic function of transient ZmWUS expression induced by TALE transcription factors was tested in regenerable tissue (see FIG. 5). Therefore, single cells of callus tissue from corn A188 were transformed by particle bombardment with the fluorescent marker tdT, TALE1 and PLT7. Induction of cell proliferation was confirmed by fluorescent microscopy upon detection of the red fluorescent signal of tdTomato (see FIG. 5, white cirle and arrow). The results clearly indicate that TALE transcription factors are able to induce regeneration and embryogenesis via transient expression of WUS and/or BBM.

Furthermore, quantitative reverse transcriptase PCR, or a western blot using a specific antibody against the ZmBBM and ZmWUS mRNA or protein, respectively, indicate the link between expression and embryogenic phenotype. The transient behavior of the expression can be detected by reverse transcriptase PCR or western blot against the ZmBBM and ZmWUS mRNA or protein respectively over time.

### Example 2: Fusion Protein Between a Non-Functional CRISPR-Nuclease and an Activation Domain for Transient Expression of Endogenous Morphogenic Genes in Zea mays

Similar to Example 1, a construct for transient delivery is designed, in this case expressing a dCas9 (PAM variants available) or dCpf1 (PAM variants available) as a fusion protein with an activation domain such as VP16 or VP64. Potential target sites/regulation regions include: Cas9 target sequences for ZmBBM set forth in SEQ ID Nos: 97 to 99; Cpf1 target sequences for ZmBBM set forth in SEQ ID Nos: 100 to 102; Cas9 target sequences for ZmWUS2 set forth in SEQ ID NOs: 103 to 105; Cpf1 target sequences for ZmWUS2 set forth in SEQ ID Nos: 106 to 108.

Based on the above described regulation regions for CRISPR/dCas9 and CRISPR/dCpf1, CRISPR based transcription factor systems can be designed and commercially obtained having a recognition domain comprising a sequence set forth in SEQ ID NOs: 1 to 12.

Transient induction of expression is first tested in maize protoplasts by PEG-mediated transformation and quantitative reverse transcriptase PCR, or western blot against the ZmBBM and ZmWUS mRNA or protein, respectively. The phenotypic function of transient ZmBBM and ZmWUS expression is then tested in regenerable tissue such as callus or immature embryos by either particle delivery or Agrobacterium mediated transformation. The successful induction of embryogenesis is recognizable by a skilled person. Furthermore, quantitative reverse transcriptase PCR, or western blot against the ZmBBM and ZmWUS mRNA or protein, respectively, indicate the link between expression and embryogenic phenotype.

The transient behavior of the expression can be detected by reverse transcriptase PCR or western blot against the ZmBBM and ZmWUS mRNA or protein respectively over time.

### Example 3: Replacement of the Activating Domain for Optimized Expression of Morphogenic Genes

This example is designed to test the behavior of different, previously described, activation domains in a systematic manner. This will allow assessing their effect on the level of expression of ZmWUS and ZmBBM. As detailed above, different STFs for a specific target gene of interest may comprise different activation and recognition domains and further elements. Therefore, it can be very suitable to design different STFs for one and the same target to ultimately define the best STF for modulating a gene of interest.

The natural activation domain of the TAL effector genes of Xanthomonas oryzae is the most obvious activation domain for use with in TAL transcription factors, and also represents one activation domain, which can be used, alone or in combination, according to the various aspects of the present invention, but have been used in other settings as well. They belong to a family of acidic (transcriptional) activation domains.

Other available activation domains have been previously tested in mammalian and insect cell systems (Chavez, Alejandro et al. “Comparative Analysis of Cas9 Activators Across Multiple Species” Nature methods 13.7 (2016): 563-567. PMC. Web. 22 Sep. 2017), but little is known about the optimum activation domains in a synthetic transcription factor to be used in a plant system, for the specific use of modulating transcription of a morphogenic gene of interest.

In this example, VP16 or VP64 in Examples 1 and 2 is replaced by either VPR, SAM, Scaffold, Suntag, P300, VP160, or a combination of at least two of these factors or VP16 and VP64 on either the N- or C-terminal or both terminal ends of the amino acid chain.

Assessment of the efficacy of activator domains in conjunction with either a TAL or dCas9 is done by quantitative reverse transcriptase PCR or western blot against the activated genes ZmBBM and ZmWUS, but it is ultimately assessed by the phenotypic response in callus or immature embryo.

### Example 4: Replacement of the Recognition Domain for Increased Targeting Variability and Flexibility

In this example, the TAL, dCas9, or dCpf1 from Examples 1, 2, and 3 are replaced with a sequence specific Zinc-Finger domain or homing endonuclease. As a fusion protein with the optimal activation domain identified in Example 3, it is possible to combine multiple transcriptional activators causing different intensities of expression for different genes. Solely relying on a dCas9 system, for example, might not allow specifically targeting of activation domains (at least for certain genes of interest) since the dCas9 or dCpf1 does not provide sufficient specificity in sgRNA binding. Specifically, dCas9 and dCpf1 systems are limited in target site specificity because they require a specific PAM motif in the regulation region of a target gene, which might not be present in at least certain genes of interest (Gao, L., et al. (2017). “Engineered Cpf1 variants with altered PAM specificities.” Nat Biotech; and Kleinstiver, B. P., et al. (2015). “Engineered CRISPR-Cas9 nucleases with altered PAM specificities.” Nature 523(7561): 481-485)). On the contrary, TAL transcription factors commonly require an initial T for target site recognition. Hence, in order to improve the binding to regulation regions of a specific target gene of interest which are difficult to access with e.g. a TAL STF, one could replace the TAL recognition domain with a dCpf1-based system in order to be able to narrow down the optimal distance to the ATG or to identify a wider target range to achieve enhanced transcriptional activation. Furthermore, the information obtained by the herein described experiments can be used to design and combine different STF systems for different endogenous regulation regions in order to improve transcriptional activation of at least one target gene of interest.

Another option to improve target site specificity and transcriptional activation is the combined use of at least two recognition domains specific for the same regulation region of the same target gene of interest (Bolukbasi, M. F., et al. (2015). “DNA-binding-domain fusions enhance the targeting range and precision of Cas9.” Nat Meth 12(12): 1150-1156).

Assessment of the additional recognition domains in conjunction with the activators from Example 3 would again be done first by quantitative reverse transcriptase PCR or western blot against the activated genes ZmBBM and ZmWUS. Ultimately, it is assessed by the phenotypic response in callus or immature embryo.

### Example 5: Morphogenic and Embryogenic Gene Targets Aside from ZmBBM and ZmWUS

Multiple genes have been described where transient overexpression in callus or immature embryos, but also leaf or other tissue, caused induction of embryogenesis. These genes or homologues thereof are individually or in a combined fashion used with the transcriptional activators in Examples 1 through 4. The list includes, but is not limited to WOX genes, other WUS and BBM homologues, Lec1 and Lec2, WIND1, ESR1, PLT3, PLT5, PLT7, IPT and IPT2, Knotted1, and RKD4. Preferably, the synthetic transcription factor designed to regulate one of the morphogenic genes disclosed herein comprises a fusion of at least two activation domains to provide for optimum recognition properties which cannot be achieved with one activation domain (e.g., dCas9 or dCpf1) alone. Furthermore, at least two activation domains properly positioned to avoid steric hindrance and to allow for a high activation rate are present.

### Example 6: Application of Transcriptional Activators for Morphogenic and Embryogenic Genes in Sugar Beet and Wheat

The processes described in Examples 1 through 5 can be transferred to all relevant crops that have a transformation protocol involving an in vitro regeneration or tissue culture step. All procedures and optimization steps as well as target genes and homologues thereof including the assessment protocols described in Examples 1 through 5 can be transferred to other crop systems. The genomic sequences of the morphogenic and embryogenic genes have to be known so that it is possible to design targets for dCas9, dCpf1 (PAM variants available for both), TAL Effectors, Zinc Fingers, and homing endonucleases can be designed and tested. Preferably, the synthetic transcription factor comprises a fusion of at least two activation domains to provide for optimum recognition properties which cannot be achieved with one activation domain (e.g., dCas9 or dCpf1) alone. Furthermore, at least two activation domains properly positioned to avoid steric hindrance and to allow for a high activation rate are present.

### Example 7: Quantitative Analysis of Increased ZmBBM and ZmWUS Transcription

The induction of BBM and WUS transcription can be measured by simple PCR system or a quantitative reverse transcriptase PCR. The advantage of the latter is the higher degree of normalization for absolute quantification of transcription. A simple PCR system would be preferably used for relative comparison of transcription against wildtype or between transformation events.

For measuring the transcriptional activation of BBM, a simple PCR assay is used. The primers are BBM-1 set forth in SEQ ID NO: 191 and BBM-2 set forth in SEQ ID NO: 192. Hot-Fire Polymerase is used in a 34 cycle PCR.

For measuring the transcriptional activation of WUS, a qRT-PCR (Taq-Man Assay) is used. The EF1 gene is used a reference. In a 40 cycle qPCR, ZmEF1 is amplified using the primers ZmEF1xxxr01 set forth in SEQ ID NO: 193 and ZmEF1xxxf01 as set forth in SEQ ID NO: 194 and detected by ZmEF1xxxMGB.1 set forth in SEQ ID NO: 195. ZmWUS is amplified using the primers WUSxxxFw1 set forth in SEQ ID NO: 196 and WUSxxxRv1 set forth in SEQ ID NO: 197 and detected by WUSxxxMGB set forth in SEQ ID NO: 198.

Statistical analysis can be performed by established and previously published methods.

### Example 8: Delivery of Synthetic Transcription Factors and Verification of Increased Morphogenesis in Corn and Sugar Beet Callus and Immature Embryos

Synthetic transcription factors as described in Examples 1 through 6 can be delivered either as DNA, RNA, or protein. Transformation of corn or sugar beet callus and immature embryos using DNA has been described and can be accomplished by either Agrobacterium tumefaciens or particle delivery. Transformation of DNA can be transient, meaning that the expression cassette is not integrated into the genome and therefore not inherited, or stable, meaning that the intention of transformation is to insert a transgene cassette. Synthetic or in vitro transcribed RNA can be delivered using bombardment. Protein delivery has been accomplished by either modified strains of Agrobacterium tumefaciens or particle delivery.

A gene or gene fragment or any other synthetic construct, e.g., including a suitable tag, transformed transiently or stably, can be introduced with or without a marker gene. Marker genes can aid in selection or screening of transformed cells or tissues. This can range from a fluorescent marker such as tdTomato to detect transformed cells to herbicide resistance genes that allow for positive selection.

A knowledgeable and skilled person can identify the effects of increased morphogenesis in corn or sugar beet tissues by eye or various forms of microscopy, i.e., by visual inspection. Typically, it is distinguishable by the increased cell division and the induction of embryogenesis in affected tissues. Embryogenesis results in the affected cells to be reprogrammed to an early embryonic developmental stage, even if they were somatic cells prior.

Depending on the effects detected, it will be potentially necessary to modify the transcription strength and expression profile to obtain the desired effect. This optimization might involve identifying the optimal transcriptional activator (Example 3), the target site (Examples 1 and 2), the promoters driving the expression, the method of delivery (Examples 8 and 10), the timing of delivery (possibility of using an inducible system), and other factors.

### Example 9: Combination of Synthetic Transcription Factors with Gene Editing for Improved Rates of Regenerated Plants Harboring Edits

The optimized transcriptional activators described in Examples 1 through 8 can be co-delivered with gene editing reagents or to T-DNA vectors. Typical transformation methods such as particle bombardment and Agrobacterium can be disadvantageous to the cells transformed or exposed. In light of the recent advances for transient activation of morphogenic genes, it is possible to co-deliver the T-DNA cassette with a plasmid containing the above described transcription factors. This gives the transformed or exposed cells an advantage instead of a disadvantage.

In this example, any plasmid encoded transient transcriptional activator from Examples 1 through 8 can be delivered by particle bombardment with an expression cassette containing a Cpf1 gene and a specifically designed crRNA (e.g. for a relevant trait gene). This cassette does not contain a resistance gene for selection. All plants regenerated from this callus are screened for the INDELs at the target site. Compared to the non-selected tissues that did not receive the transcriptional activator, we would expect the INDEL efficiency to be significantly lower.

Taking the successful edited plants to the next generation and reconfirming the modification by Cpf1 or other site-directed nucleases, we would expect to have higher counts of edited T1 plants than in the control.

### Example 10: Protein-Based Co-Delivery of Synthetic Transcriptional Activators with Site-Directed Nuclease RNPs for Improved Transient Gene Editing

In this example, the components of Example 9 are delivered into plant tissue such as callus or immature embryo as purified protein. The transcription factors described in Examples 1 through 8 are expressed in and purified from a pro- or eukaryotic cell system. Cpf1 is equally produced and incubated with synthetic or in vitro transcribed crRNA to form ribonucleoprotein (RNP). Protein delivery has been demonstrated by particle bombardment or fusion to cell penetrating peptides. It would be expected to get lower counts of edited T1 plants compared to Example 9. However, the complete absence of heritable material makes this approach highly desirable.

### Example 11: Combination of Synthetic Transcription Factors with Base Editing for Improved Rates of Regenerated Plants Harboring Edits

The optimized transcriptional activators described in Examples 1 through 8 are co-delivered with base editing reagents on co-bombarded DNA cassettes or on one or more T-DNA vectors harboring their expression cassettes. Typical transformation methods such as particle bombardment and Agrobacterium can be disadvantageous to the cells transformed or exposed. In light of the recent advances for transient activation of morphogenic genes, it is possible to co-deliver the T-DNA cassette with a plasmid containing the above described transcription factors. This gives the transformed or exposed cells an advantage instead of a disadvantage.

In this example, any plasmid-encoded transcriptional activator from Examples 1 through 8 can be delivered by particle bombardment with an expression cassette containing a base editor gene and a specifically designed guide RNA (e.g. for a relevant trait gene) to direct the base editor to the appropriate target. This cassette may or may not contain a resistance gene for selection. The base editor gene can encode a cytidine deaminase, an adenine deaminese, or another deaminase or other catalytic activity suitable for making base conversions. The base editor can further be based on any CRISPR domain suitable for delivering the base editing function to the target site. This can include, but is not limited to, Cas9, Cpf1, CasX, CasY, or other suitable domains. All plants regenerated from this callus are screened for base substitutions at the target site. Compared to cells that did not receive the transcriptional activator(s), we would expect the regeneration efficiency to be much higher.

### Example 12: Protein-Based Co-Delivery of Synthetic Transcriptional Activators with Base Editor RNPs for Improved Transient Gene Editing

In this example, the components of Example 11 are delivered into plant tissue such as callus or immature embryo as purified protein and RNA. The transcription factors described in Examples 1 through 8 are expressed in and purified from a pro- or eukaryotic cell system. The base editor is equally produced and incubated with synthetic or in vitro transcribed crRNA to form ribonucleoprotein (RNP). Protein delivery has been demonstrated by particle bombardment or fusion to cell penetrating peptides. It would be expected to get lower counts of edited T1 plants compared to Example 11. However, the complete absence of heritable material makes this approach highly desirable.

### Example 13: Generation of a Cpf1-Based Transcriptional Activator

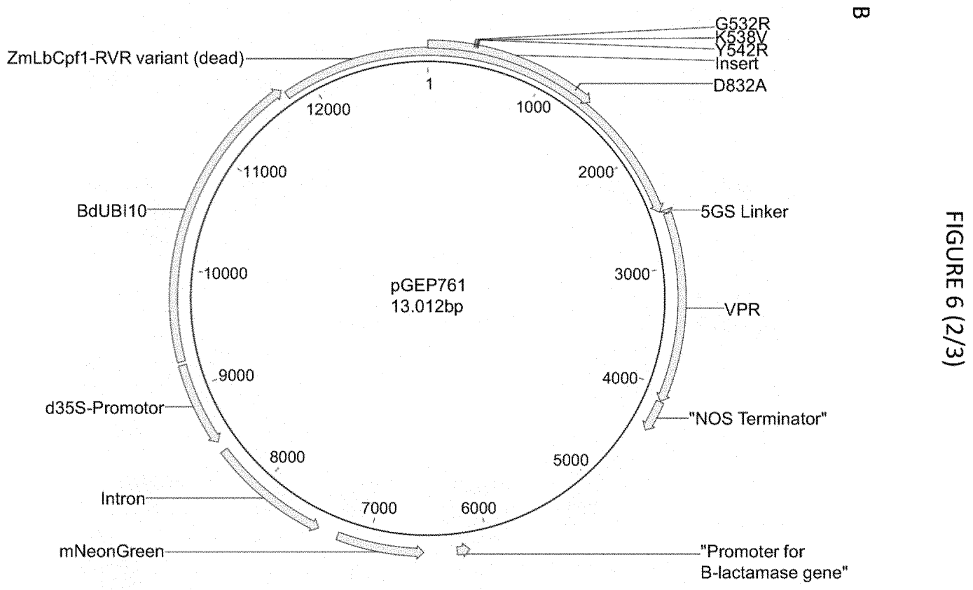

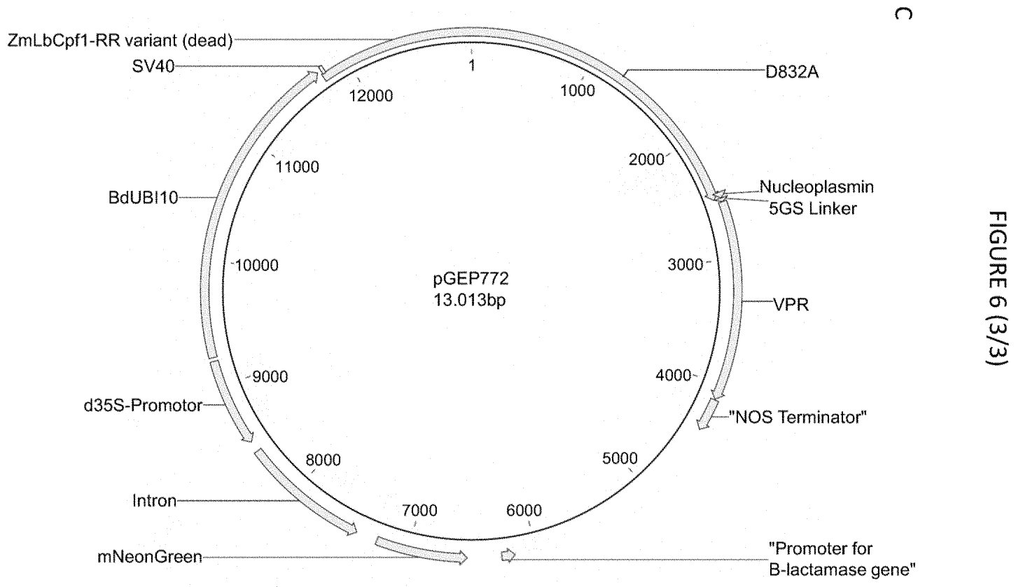

For the generation of a Cpf1-based transcriptional activator LbCpf1 expression plasmids were used including the wild type Lbcpf1 recognizing the original TTTV PAM motif (pGEP362, SEQ ID NO: 273), and two LbCpf1 variants (RR and RVR) that recognize the TYCV and TATV PAM motifs, respectively (pGEP487, SEQ ID NO: 274; and pGEP488, SEQ ID NO: 275). Besides the LbCpfs encoding polynucleotide, these constructs further contain a fluorescent marker mNeoGreen (see FIG. 6 A-C). To obtain a Cpf1-based transcriptional activator, the VPR transcriptional activation domain (SEQ ID NO: 276) was first fused to the C-terminus of LbCpf1. It was shown in mammalian cells that dAsCpf1-VP64 fusion only resulted in minimal activation when used to activate GFP expression, whereas use of the VPR activation domain resulted in over 20-fold of transcriptional activation (see Liu et al. (2017), supra). Furthermore, the dCAs9-VP64 fusion construct also only showed weak activation of target genes with a single sgRNA (in some cases even with multiple sgRNAs) in plant and animal cells. Based on these observations, the VPR activation domain was used, which was demonstrated to induce robust transcriptional activation in mammalian cells with dCpf1-VPR fusion systems (Liu et al. (2017), supra; and Tak et al. (2017), supra).

The sequence of the VPR domain (SEQ ID NO: 276) used in Tak et al. (2017) was adapted and a 5×GS linker (SEQ ID NO: 277), which was employed in Cas9-based plant transcription activation systems (Lowder et al. (2017), supra) was used between the LbCpf1 and the VPR domain. The DNA sequence encoding the 5×GS linker and the VPR domain was codon optimized for maize (service from Genscript). To facilitate the cloning process, the codon-optimized sequence was synthesized by Genscript flanked by the 3′end of the LbCpf1coding region at the 5′end and the Nos terminator at the 3′end in the pUC57 cloning vector between EcoRI and HindIII restriction sites. The resulting plasmid was named pKWS20 and is set forth in (SEQ ID NO: 278).

Next, the fragment of 5×GS linker with VPR domain followed by the Nos terminator in the pKWS20 was released by EcoRI and HindIII double digestion and cloned into the backbone of MscI and XmaI double digested pGEP362 (SEQ ID NO: 273), pGEP487 (SEQ ID NO: 274) or pGEP488 (SEQ ID NO: 275) with Gibson assembly to produce pGEP754 (SEQ ID NO: 279), pGEP755 (SEQ ID NO: 280) and pGEP756 (SEQ ID NO: 281), harboring the wild type LbCpf1 (SEQ ID NO: 282) or RR variant of LbCpf1 (LbCpf1(RR), SEQ ID NO: 283) or RVR variant of LbCpf1(LbCpf1-RVR, SEQ ID NO: 284) fused with VPR activation domain. A D832A mutation was further introduced in pGEP754, pGEP755 and pGEP756 to produce the pGEP767 (SEQ ID NO: 285), pGEP772 (SEQ ID NO: 286) and pGEP761(SEQ ID NO: 287), which contains dLbCpf1-VPR (SEQ ID NO: 288), or dLbCpf1(RR)-VPR (SEQ ID NO: 289) or dLbCpf1(RVR)-VPR (SEQ ID NO: 290) expression cassettes respectively. Plasmids pGEP767, pGEP772 and pGEP761 (FIG. 6A, B, C) were used in the following transcriptional activation experiments in combination with different guide RNA expressing plasmids.

### Example 14: Guide RNA Design for Targeting BBM and WUS

Maize Babyboom (BBM, SEQ ID NO: 307) and Wuschel 2 (WUS2, SEQ ID NO: 308) genes are morphogenic genes that have been reported to produce high transformation frequencies in numerous previously non-transformable maize inbred lines through heterologous overexpression (Lowe et al., 2016, supra). In order to test whether activation of the endogenous BBM and WUS2 gene expression would have a similar effect, guide RNAs are designed targeting BBM (SEQ ID NO: 295-298) and WUS2 (SEQ ID NO: 291-294) promoter regions to be combined with LbCpf1-VPR fusion proteins.

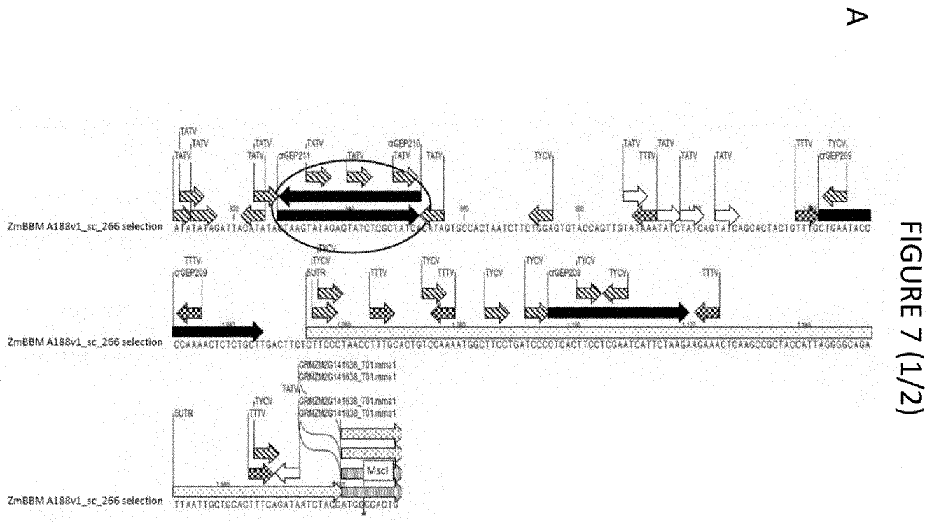

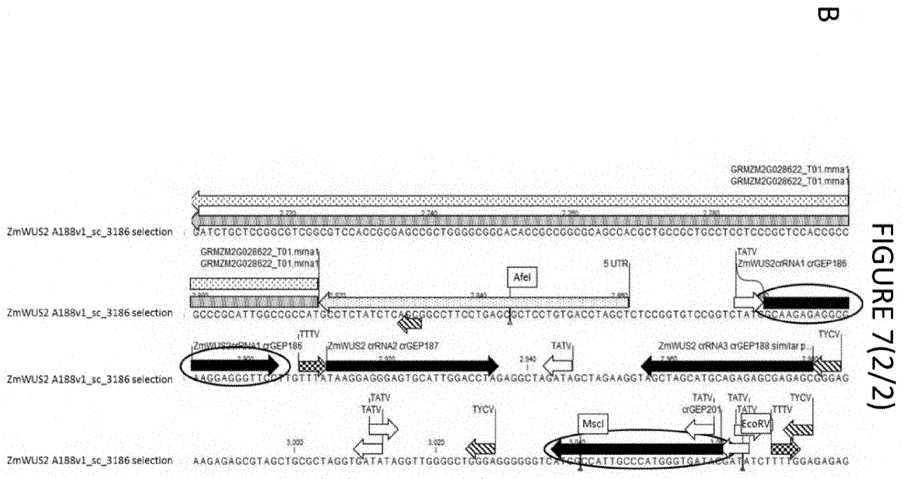

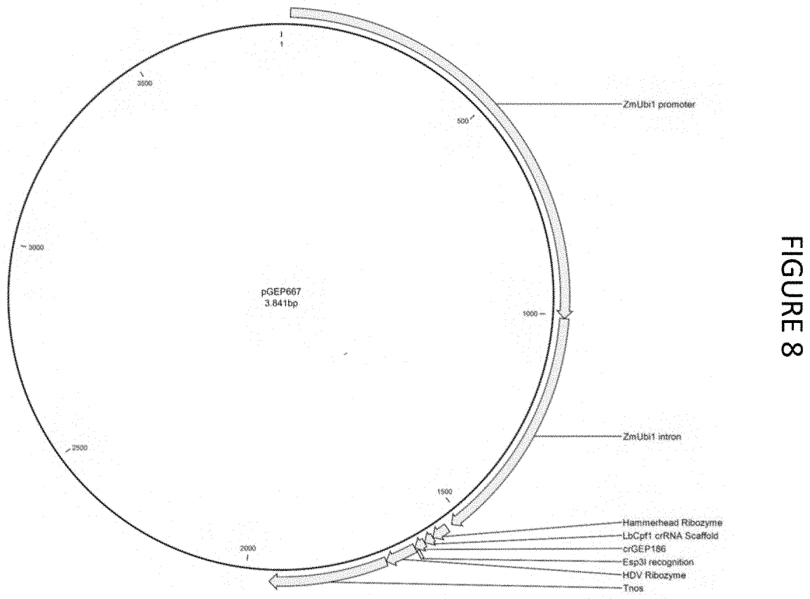

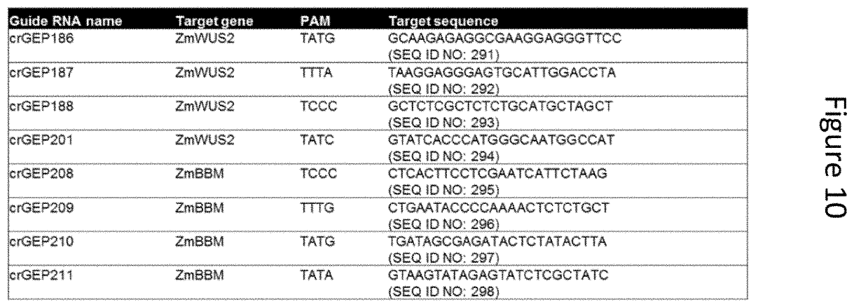

It is reported that using the dCpf1-VPR fusion system in mammalian cells, transcriptional activation was detected with targets between ˜600 bp upstream and −400 bp downstream of the transcription start sites (Tak et al. (2017), supra). Based on this, the promoter regions of ZmBBM and ZmWUS2 were scanned for all possible PAMs from ˜500 bp upstream of the transcription start sites to the translation start sites and a total of 4 guide RNAs for BBM (SEQ ID NO: 295-298) and 4 guide RNAs for WUS2 (SEQ ID NO: 291-294), using different PAMs, were designed spanning the whole area (FIG. 7 and FIG. 10). For each guide RNA sequence, complementary oligo sets were synthesized from IDT, annealed and cloned into pGEP296 (SEQ ID NO: 299-306) between the LbCpf1 crRNA scaffold and hepatitis delta virus (HDV) ribozymes through Golden Gate Assembly (see FIG. 8 for a representative plasmid map).

### Example 15: Transcriptional Regulation of ZmBBM and ZmWUS2 Using LbCpf1-VPR System

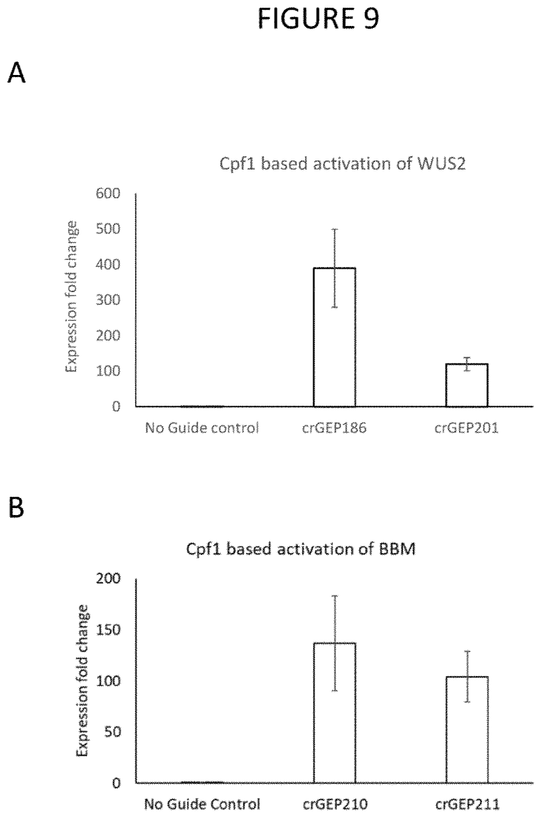

Transient activation of endogenous gene expression is first tested in maize protoplasts by PEG-mediated transformation followed by quantitative reverse transcription-PCR. To do this, 15 μg plasmid DNA encoding the LbCpf1-VPR fusion protein and 8 μg plasmid DNA expressing the guide RNA were co-delivered to approximately 600,000 maize protoplasts via a PEG-based transformation system commonly known in the art. 24 hours after transformation, protoplast samples were collected for RNA extraction and cDNA synthesis using a commercially available kit. Expression of endogenous ZmBBM and ZmWUS2 was then determined using a SYBR Green qRT-PCR approach. As shown in FIG. 9, the tested guide RNAs targeting the promoter region of WUS2crGEP186 (SEQ ID NO: 291) and crGEP201 (SEQ ID NO: 294) resulted in significant activation of WUS2 expression (FIG. 9A). Similarly, the guide RNAs targeting the BBM promoter region crGEP210 (SEQ ID NO: 297) and crGEP211 (SEQ ID NO: 298) were found to cause robust activation of endogenous BBM (FIG. 9B). Since this experiment has been done with only one biological replicate (three technical replicates), further confirmation is needed and experiments are undergoing. Nevertheless, the data presented herein for the first time clearly indicate that Cpf1-based transcriptional activation systems can be used in order to stimulate gene activation in plants.

1. A synthetic transcription factor, or a nucleotide sequence encoding the same, comprising at least one recognition domain and at least one activation domain, wherein the synthetic transcription factor is configured to modulate the expression of a morphogenic gene in a cellular system.

2. A synthetic transcription factor, or a nucleotide sequence encoding the same, comprising at least one recognition domain and at least one activation domain, wherein the synthetic transcription factor is configured to activate the expression of an endogenous gene in a cellular system.

3. The synthetic transcription factor of claim 1, wherein the at least one recognition domain is, or is a fragment of at least one disarmed CRISPR/nuclease system.

4. The synthetic transcription factor of claim 3, wherein the at least one disarmed CRISPR/nuclease system is a CRISPR/dCpf1 system, wherein the at least one disarmed CRISPR/nuclease system comprises at least one guide RNA.

5. The synthetic transcription factor of claim 1, wherein the at least one activation domain is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof.

6. The synthetic transcription factor of claim 1, wherein the at least one activation domain is located N-terminal and/or C-terminal relative to the at least one recognition domain.

7. The synthetic transcription factor of claim 1, wherein the morphogenic gene is selected from the group consisting of BBM, WUS, including WUS2, a WOX gene, a WUS or BBM homologue, Lec1, Lec2, WIND1, ESR1, PLT3, 5, or 7, IPT, IPT2, Knotted1, and RKD4.

8. The synthetic transcription factor of claim 1, wherein the synthetic transcription factor is configured to modulate expression, preferably transcription, of the morphogenic gene by binding to a regulation region located at a certain distance in relation to the start codon.

9. The synthetic transcription factor of claim 2, wherein the endogenous gene is selected from the group consisting of a gene encoding resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, resistance or tolerance to 2,4-D, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content.

10. The synthetic transcription factor of claim 1, wherein the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

11. The synthetic transcription factor of claim 1, wherein the cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

12. The synthetic transcription factor of claim 11, wherein the at least one part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

13. The synthetic transcription factor of claim 12, wherein the at least one plant cell, the at least one plant or the at least one part of a plant originates from a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

14. A method for increasing the transformation efficiency in a cellular system, wherein the method comprises the steps of:
(a) providing a cellular system;
(b) introducing into the cellular system at least one synthetic transcription factor, or a nucleotide sequence encoding the same; and
(c) introducing into the cellular system at least one nucleotide sequence of interest;
(d) optionally: culturing the cellular system under conditions to obtain a transformed progeny of the cellular system;
wherein the at least one synthetic transcription factor, or the nucleotide sequence encoding the same, comprises at least one recognition domain and at least one activation domain, wherein the synthetic transcription factor is configured to modulate the expression, preferably the transcription, of at least one morphogenic gene in the cellular system; and
wherein the at least one synthetic transcription factor, or the nucleotide sequence encoding the same, is introduced in parallel to, or sequentially with the introduction of the at least one nucleotide sequence of interest.

15. The method of claim 14, wherein
(a) the at least one synthetic transcription factor, or the sequence encoding the same, or at least one component of the at least one synthetic transcription factor, or the sequence encoding the same; and
(b) the at least one nucleotide sequence of interest
is/are introduced into the cellular system by means independently selected from biological and/or physical means, including transfection, transformation, including transformation by Agrobacterium spp., preferably, Agrobacterium tumefaciens, a viral vector, biolistic bombardment, transfection using chemical agents, including polyethylene glycol transfection, or any combination thereof.

16. A method for increasing the expression of at least one endogenous gene in a cellular system, wherein the method comprises the steps of:
(a) providing a cellular system;
(b) introducing into the cellular system at least one synthetic transcription factor, or a nucleotide sequence encoding the same;
wherein the at least one synthetic transcription factor, or the nucleotide sequence encoding the same, comprises at least one recognition domain and at least one activation domain, wherein the synthetic transcription factor is configured to increase the expression, preferably the transcription, of at least one endogenous gene in the cellular system.

17. The method of claim 16, wherein the at least one synthetic transcription factor, or the sequence encoding the same, or at least one component of the at least one synthetic transcription factor, or the sequence encoding the same is introduced into the cellular system by means independently selected from biological and/or physical means, including transfection, transformation, including transformation by Agrobacterium spp., preferably, Agrobacterium tumefaciens, a viral vector, biolistic bombardment, transfection using chemical agents, including polyethylene glycol transfection, or any combination thereof.

18. The method of claim 14, wherein the at least one recognition domain is or is a fragment of at least one disarmed non-functional CRISPR/nuclease system.

19. The method of claim 18, wherein the at least one disarmed CRISPR/nuclease system is a CRISPR/dCpf1 system, wherein the at least one disarmed CRISPR/nuclease system comprises at least one guide RNA.

20. The method of claim 14, wherein the at least one activation domain of the at least one synthetic transcription factor is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof.

21. The method of claim 14, wherein the at least one activation domain of the at least one synthetic transcription factor is located N-terminal and/or C-terminal relative to the at least one recognition domain of the at least one synthetic transcription factor.

22. The method of claim 14, wherein the at least one morphogenic gene is selected from the group consisting of BBM, WUS, including WUS2, a WOX gene, a WUS or BBM homologue, Lec1, Lec2, WIND1, ESR1, PLT3, 5, or 7, IPT, IPT2, Knotted1, and RKD4.

23. The method of claim 14, wherein the synthetic transcription factor is configured to modulate expression, preferably transcription, of the morphogenic gene by binding to a regulation region located at a certain distance in relation to the start codon.

24. The method of claim 16, wherein the endogenous gene is selected from the group consisting of a gene encoding resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, resistance or tolerance to 2,4-D, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content.

25. The method of claim 14, wherein the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

26. The method of claim 14, wherein the cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

27. The method of claim 26, wherein the at least one part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

28. The method of claim 27, wherein the at least one plant cell, the at least one plant or the at least one part of a plant originates from a plant species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

29. A method of modifying the genetic material of a cellular system at a predetermined location, wherein the method comprises the following steps:
(a) providing a cellular system;
(b) introducing at least one synthetic transcription factor, or a sequence encoding the same, into the cellular system,
(c) further introducing into the cellular system
(i) at least one site-specific nuclease, or a sequence encoding the same, wherein the site-specific nuclease induces a double-strand break at the predetermined location;
(ii) optionally: at least one nucleotide sequence of interest, preferably flanked by one or more homology sequence(s) complementary to one or more nucleotide sequence(s) adjacent to the predetermined location in the genetic material of the cellular system; and;
(e) optionally: determining the presence of the modification at the predetermined location in the genetic material of the cellular system; and
(f) obtaining a cellular system comprising a modification at the predetermined location of the genetic material of the cellular system;
wherein the at least one synthetic transcription factor, or the nucleotide sequence encoding the same, comprises at least one recognition domain and at least one activation domain, wherein the at least one synthetic transcription factor is configured to modulate the expression, preferably the transcription, of at least one morphogenic gene in the cellular system; and
wherein the at least one synthetic transcription factor, or the nucleotide sequence encoding the same, is introduced in parallel to, or sequentially with the introduction of the at least one site-specific nuclease, or the sequence encoding the same and the optional at least one nucleotide sequence of interest.

30. The method of claim 29, wherein the method further comprises the step of culturing the cellular system under conditions to obtain a genetically modified progeny of the modified cellular system.

31. The method of claim 29, wherein
(i) the at least one synthetic transcription factor, or the sequence encoding the same, or at least one component of the at least one synthetic transcription factor, or the sequence encoding the same; and
(ii) the at least one site-specific nuclease, or the sequence including the same; and optionally
(iii) the at least one nucleotide sequence of interest
is/are introduced into the cellular system by means independently selected from biological and/or physical means, including transfection, transformation, including transformation by Agrobacterium spp. transformation, preferably by Agrobacterium tumefaciens, a viral vector, biolistic bombardment, transfection using chemical agents, including polyethylene glycol transfection, or any combination thereof.

32. The method of claim 29, wherein the at least one recognition domain is or is a fragment of least one disarmed CRISPR/nuclease system.

33. The method of claim 32, wherein the at least one disarmed CRISPR/nuclease system is a CRISPR/dCpf1 system, wherein the at least one disarmed CRISPR/nuclease system comprises at least one guide RNA.

34. The method of claim 29, wherein the at least one activation domain of the at least one synthetic transcription factor is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof.

35. The method of claim 29, wherein the at least one activation domain of the at least one synthetic transcription factor is located N-terminal and/or C-terminal relative to the at least one recognition domain of the at least one synthetic transcription factor.

36. The method of claim 29, wherein the at least one morphogenic gene is selected from the group consisting of BBM, WUS, including WUS2, a WOX gene, a WUS or BBM homologue, Lec1, Lec2, WIND1, ESR1, PLT3, 5, or 7, IPT, IPT2, Knotted1, and RKD4.

37. The method of claim 29, wherein the synthetic transcription factor is configured to modulate expression, preferably transcription, of the morphogenic gene by binding to a regulation region located at a certain distance in relation to the start codon.

38. The method of claim 29, wherein the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

39. The method of claim 29, wherein the cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

40. The method of claim 29, wherein the one or more nucleotide sequence(s) flanking the at least one nucleotide sequence of interest at the predetermined location is/are at least 85%-100% complementary to the one or more nucleotide sequence(s) adjacent to the predetermined location, upstream and/or downstream from the predetermined location, over the entire length of the respective adjacent region(s).

41. A method of producing a haploid or double haploid organism, wherein the method comprises the following steps:
(a) providing a haploid cellular system;
(b) introducing into the haploid cellular system at least one synthetic transcription factor, or a nucleotide sequence encoding the same;
(c) culturing the haploid cellular system under conditions to obtain at least one haploid or double haploid organism; and
(d) optionally: selecting the at least one haploid or double haploid organism obtained in step (c),
wherein the at least one synthetic transcription factor, or the nucleotide sequence encoding the same, comprises at least one recognition domain and at least one activation domain, wherein the at least one synthetic transcription factor is configured to modulate the expression, preferably the transcription, of at least one morphogenic gene in the haploid cellular system.

42. The method of claim 41, wherein the haploid cellular system of step (a) is a haploid embryo, or wherein the at least one haploid or double haploid organism defined in step (c) is obtained through an intermediate step of generating at least one haploid embryo from the haploid cellular system of (b).

43. The method of claim 41, wherein the at least one synthetic transcription factor, or a sequence encoding the same, or at least one component of the at least one synthetic transcription factor, or the sequence encoding the same is/are introduced into the haploid cellular system by means independently selected from biological and/or physical means, including transfection, transformation, including transformation by Agrobacterium spp. transformation, preferably by Agrobacterium tumefaciens, a viral vector, biolistic bombardment, transfection using chemical agents, including polyethylene glycol transfection, or any combination thereof.

44. The method of claim 41, wherein the at least one recognition domain is or is a fragment of at least one disarmed CRISPR/nuclease system.

45. The method of claim 44, wherein the at least one disarmed CRISPR/nuclease system is a CRISPR/dCpf1 system, wherein the at least one disarmed CRISPR/nuclease system comprises at least one guide RNA.

46. The method of claim 41 wherein the at least one activation domain of the at least one synthetic transcription factor is selected from the group consisting of an acidic transcriptional activation domain, preferably, wherein the at least one activation domain is from an avirulence gene of Xanthomonas oryzae, VP16 or tetrameric VP64 from Herpes simplex, VPR, SAM, Scaffold, Suntag, P300, VP160, or any combination thereof.

47. The method of claim 41, wherein the at least one activation domain of the at least one synthetic transcription factor is located N-terminal and/or C-terminal relative to the at least one recognition domain of the at least one synthetic transcription factor.

48. The method of claim 41, wherein the at least one morphogenic gene is selected from the group consisting of BBM, WUS, including WUS2, a WOX gene, a WUS or BBM homologue, Lec1, Lec2, WIND1, ESR1, PLT3, 5, or 7, IPT, IPT2, Knotted1, and RKD4.

49. The method of claim 41, wherein the synthetic transcription factor is configured to modulate expression, preferably transcription, of the morphogenic gene by binding to a regulation region located at a certain distance in relation to the start codon.

50. The method of claim 41, wherein the synthetic transcription factor and/or the at least one recognition domain comprises a sequence set forth in any one of SEQ ID NOs: 276, 277, 282, 283, 284, 288, 289, 290, or a sequence having at least 85%, 86%, 87%, 88%, 89%, 90%, 91%, 92%, 93%, 94%, 95%, 96%, 97%, 98%, or 99% identity over the whole length of any one of SEQ ID NOs: 276, 277, 282, 283, 284, 288, 289, 290.

51. The method of claim 41, wherein the at least one haploid cellular system is selected from the group consisting of at least one eukaryotic cell or eukaryotic organism, preferably wherein the at least one eukaryotic cell is at least one plant cell, and/or wherein the at least one eukaryotic organism is a plant or a part of a plant.

52. A cellular system or a progeny thereof obtained by a method of claim 14.

53. A cellular system or a progeny thereof obtained by a method of claim 29.

54. A haploid or double haploid organism obtained by the method of claim 41.

55. A use of a synthetic transcription factor of claim 1 in a method for increasing the transformation efficiency in a cellular system, wherein the method comprises the steps of:
(a) providing a cellular system;
(b) introducing into the cellular system at least one synthetic transcription factor, or a nucleotide sequence encoding the same; and
(c) introducing into the cellular system at least one nucleotide sequence of interest;
(d) optionally: culturing the cellular system under conditions to obtain a transformed progeny of the cellular system;
wherein the at least one synthetic transcription factor, or the nucleotide sequence encoding the same, comprises at least one recognition domain and at least one activation domain, wherein the synthetic transcription factor is configured to modulate the expression, preferably the transcription, of at least one morphogenic gene in the cellular system; and
wherein the at least one synthetic transcription factor, or the nucleotide sequence encoding the same, is introduced in parallel to, or sequentially with the introduction of the at least one nucleotide sequence of interest.

56. A use of a synthetic transcription factor of claim 1 in a method of modifying the genetic material of a cellular system at a predetermined location, wherein the method comprises the following steps:
(a) providing a cellular system;
(b) introducing at least one synthetic transcription factor, or a sequence encoding the same, into the cellular system,
(c) further introducing into the cellular system
(i) at least one site-specific nuclease, or a sequence encoding the same, wherein the site-specific nuclease induces a double-strand break at the predetermined location;
(ii) optionally: at least one nucleotide sequence of interest, preferably flanked by one or more homology sequence(s) complementary to one or more nucleotide sequence(s) adjacent to the predetermined location in the genetic material of the cellular system; and;
(e) optionally: determining the presence of the modification at the predetermined location in the genetic material of the cellular system; and
(f) obtaining a cellular system comprising a modification at the predetermined location of the genetic material of the cellular system;
wherein the at least one synthetic transcription factor, or the nucleotide sequence encoding the same, comprises at least one recognition domain and at least one activation domain, wherein the at least one synthetic transcription factor is configured to modulate the expression, preferably the transcription, of at least one morphogenic gene in the cellular system; and
wherein the at least one synthetic transcription factor, or the nucleotide sequence encoding the same, is introduced in parallel to, or sequentially with the introduction of the at least one site-specific nuclease, or the sequence encoding the same and the optional at least one nucleotide sequence of interest.

57. A use of a synthetic transcription factor of claim 1 in a method of producing a haploid or double haploid organism, wherein the method comprises the following steps:
(a) providing a haploid cellular system;
(b) introducing into the haploid cellular system at least one synthetic transcription factor, or a nucleotide sequence encoding the same;
(c) culturing the haploid cellular system under conditions to obtain at least one haploid or double haploid organism; and
(d) optionally: selecting the at least one haploid or double haploid organism obtained in step (c),
wherein the at least one synthetic transcription factor, or the nucleotide sequence encoding the same, comprises at least one recognition domain and at least one activation domain, wherein the at least one synthetic transcription factor is configured to modulate the expression, preferably the transcription, of at least one morphogenic gene in the haploid cellular system.

58. A use of a synthetic transcription factor of claim 2 in a method for increasing the expression of at least one endogenous gene in a cellular system, wherein the method comprises the steps of:
(a) providing a cellular system;
(b) introducing into the cellular system at least one synthetic transcription factor, or a nucleotide sequence encoding the same;
wherein the at least one synthetic transcription factor, or the nucleotide sequence encoding the same, comprises at least one recognition domain and at least one activation domain, wherein the synthetic transcription factor is configured to increase the expression, preferably the transcription, of at least one endogenous gene in the cellular system.


SEQUENCE LISTING (318 sequences)

SEQ ID NO: 1 - RNA, 100 nt, Artificial Sequence
  synthetic construct
caccgcucugaucacaagcaguuuuagagcuagaaauagcaaguuaaaauaaggcuaguccguuaucaacuugaaaaaguggcaccgagucggugcuuuu

SEQ ID NO: 2 - RNA, 100 nt, Artificial Sequence
  synthetic construct
cccauguguuguucuaucccguuuuagagcuagaaauagcaaguuaaaauaaggcuaguccguuaucaacuugaaaaaguggcaccgagucggugcuuuu

SEQ ID NO: 3 - RNA, 100 nt, Artificial Sequence
  synthetic construct
acacaugggucagugugaagguuuuagagcuagaaauagcaaguuaaaauaaggcuaguccguuaucaacuugaaaaaguggcaccgagucggugcuuuu

SEQ ID NO: 4 - RNA, 100 nt, Artificial Sequence
  synthetic construct
gucuauggcaagagaggcgaguuuuagagcuagaaauagcaaguuaaaauaaggcuaguccguuaucaacuugaaaaaguggcaccgagucggugcuuuu

SEQ ID NO: 5 - RNA, 100 nt, Artificial Sequence
  synthetic construct
uuuauaaggagggagugcauguuuuagagcuagaaauagcaaguuaaaauaaggcuaguccguuaucaacuugaaaaaguggcaccgagucggugcuuuu

SEQ ID NO: 6 - RNA, 100 nt, Artificial Sequence
  synthetic construct
uagcaugcagagagcgagagguuuuagagcuagaaauagcaaguuaaaauaaggcuaguccguuaucaacuugaaaaaguggcaccgagucggugcuuuu

SEQ ID NO: 7 - RNA, 45 nt, Artificial Sequence
  synthetic construct
uaauuucuacuaaguguagauaccgcucugaucacaagcaaggca

SEQ ID NO: 8 - RNA, 45 nt, Artificial Sequence
  synthetic construct
uaauuucuacuaaguguagauuggaaagcuauaccuccuuacccc

SEQ ID NO: 9 - RNA, 45 nt, Artificial Sequence
  synthetic construct
uaauuucuacuaaguguagauugcccucuucacacugacccaugu

SEQ ID NO: 10 - RNA, 45 nt, Artificial Sequence
  synthetic construct
uaauuucuacuaaguguagaugcaagagaggcgaaggaggguucc

SEQ ID NO: 11 - RNA, 45 nt, Artificial Sequence
  synthetic construct
uaauuucuacuaaguguagauuaaggagggagugcauuggaccua

SEQ ID NO: 12 - RNA, 45 nt, Artificial Sequence
  synthetic construct
uaauuucuacuaaguguagaugcucucgcucucugcaugcuagcu

SEQ ID NO: 13 - protein, 36 aa, Artificial Sequence
  recognition domain
HDHDNGNHHDHDHDNGHDNGNGHDNIHDNIHDNGNH

SEQ ID NO: 14 - protein, 36 aa, Artificial Sequence
  recognition domain
HDNGNGNGNINGHDHDNGNGNINININGNININHNI

SEQ ID NO: 15 - protein, 36 aa, Artificial Sequence
  recognition domain
NHHDHDHDNGHDNGNGHDNIHDNIHDNGNHNIHDHD

SEQ ID NO: 16 - protein, 36 aa, Artificial Sequence
  recognition domain
NINGHDHDNGNGNINININGNININHNININHHDNI

SEQ ID NO: 17 - protein, 36 aa, Artificial Sequence
  recognition domain
HDHDNGNGNINININGNININHNININHHDNINGNI

SEQ ID NO: 18 - protein, 36 aa, Artificial Sequence
  recognition domain
HDNGNGHDNIHDNIHDNGNHNIHDHDHDNINGNHNG

SEQ ID NO: 19 - protein, 36 aa, Artificial Sequence
  recognition domain
NHNGNGHDNGNINGHDNINIHDNHHDHDHDHDNGHD

SEQ ID NO: 20 - protein, 36 aa, Artificial Sequence
  recognition domain
HDNGNINGHDNINIHDNHHDHDHDHDNGHDHDHDNG

SEQ ID NO: 21 - protein, 36 aa, Artificial Sequence
  recognition domain
NINGHDNINIHDNHHDHDHDHDNGHDHDHDNGNGNI

SEQ ID NO: 22 - protein, 36 aa, Artificial Sequence
  recognition domain
NHNGNGNHNGNGHDNGNINGHDHDHDNGNHNHNINI

SEQ ID NO: 23 - protein, 36 aa, Artificial Sequence
  recognition domain
NHNGNGHDNGNINGHDHDHDNGNHNHNINININHHD

SEQ ID NO: 24 - protein, 36 aa, Artificial Sequence
  recognition domain
HDNGNINGHDHDHDNGNHNHNINININHHDNGNING

SEQ ID NO: 25 - protein, 36 aa, Artificial Sequence
  recognition domain
NINGHDHDHDNGNHNHNINININHHDNGNINGNIHD

SEQ ID NO: 26 - protein, 36 aa, Artificial Sequence
  recognition domain
HDNGHDNINHHDHDNINHNGNGHDNGNGNINIHDNG

SEQ ID NO: 27 - protein, 36 aa, Artificial Sequence
  recognition domain
NHNHNINININHHDNGNINGNIHDHDNGHDHDNGNG

SEQ ID NO: 28 - protein, 36 aa, Artificial Sequence
  recognition domain
NINGNIHDHDNGHDHDNGNGNIHDHDHDHDNGNING

SEQ ID NO: 29 - protein, 36 aa, Artificial Sequence
  recognition domain
NIHDHDNGHDHDNGNGNIHDHDHDHDNGNINGHDNI

SEQ ID NO: 30 - protein, 36 aa, Artificial Sequence
  recognition domain
HDHDNGNGNIHDHDHDHDNGNINGHDNINHHDNGNG

SEQ ID NO: 31 - protein, 36 aa, Artificial Sequence
  recognition domain
HDNGHDNGNGNINGNINININGNIHDNINHNIHDHD

SEQ ID NO: 32 - protein, 36 aa, Artificial Sequence
  recognition domain
HDNGNGNINGNINININGNIHDNINHNIHDHDNGNG

SEQ ID NO: 33 - protein, 36 aa, Artificial Sequence
  recognition domain
NIHDHDHDHDNGNINGHDNINHHDNGNGHDNGHDHD

SEQ ID NO: 34 - protein, 36 aa, Artificial Sequence
  recognition domain
NINGNINININGNIHDNINHNIHDHDNGNGNHNGNI

SEQ ID NO: 35 - protein, 36 aa, Artificial Sequence
  recognition domain
NINGHDNINHHDNGNGHDNGHDHDNGHDNIHDNING

SEQ ID NO: 36 - protein, 36 aa, Artificial Sequence
  recognition domain
NIHDNINHNIHDHDNGNGNHNGNIHDNINIHDNIHD

SEQ ID NO: 37 - protein, 36 aa, Artificial Sequence
  recognition domain
HDNGHDHDNGHDNIHDNINGHDNGHDHDNGHDNGHD

SEQ ID NO: 38 - protein, 36 aa, Artificial Sequence
  recognition domain
HDHDNGHDNIHDNINGHDNGHDHDNGHDNGHDNHNG

SEQ ID NO: 39 - protein, 36 aa, Artificial Sequence
  recognition domain
NHNGNIHDNINIHDNIHDNGNGNGHDNIHDHDNGHD

SEQ ID NO: 40 - protein, 36 aa, Artificial Sequence
  recognition domain
NIHDNINIHDNIHDNGNGNGHDNIHDHDNGHDHDNG

SEQ ID NO: 41 - protein, 36 aa, Artificial Sequence
  recognition domain
HDNGHDHDNGHDNGHDNHNGHDNHHDHDNIHDHDHD

SEQ ID NO: 42 - protein, 36 aa, Artificial Sequence
  recognition domain
HDHDNGHDNGHDNHNGHDNHHDHDNIHDHDHDNING

SEQ ID NO: 43 - protein, 36 aa, Artificial Sequence
  recognition domain
HDNGHDNHNGHDNHHDHDNIHDHDHDNINGNHHDNG

SEQ ID NO: 44 - protein, 36 aa, Artificial Sequence
  recognition domain
HDNHNGHDNHHDHDNIHDHDHDNINGNHHDNGNING

SEQ ID NO: 45 - protein, 36 aa, Artificial Sequence
  recognition domain
HDNHHDHDNIHDHDHDNINGNHHDNGNINGHDNIHD

SEQ ID NO: 46 - protein, 36 aa, Artificial Sequence
  recognition domain
HDHDHDNGNHNHNINININHHDNGNINGNIHDHDNG

SEQ ID NO: 47 - protein, 36 aa, Artificial Sequence
  recognition domain
NHHDNGNINGHDNIHDHDNHHDNGHDNGNHNINGHD

SEQ ID NO: 48 - protein, 36 aa, Artificial Sequence
  recognition domain
NINGHDNIHDHDNHHDNGHDNGNHNINGHDNIHDNI

SEQ ID NO: 49 - protein, 36 aa, Artificial Sequence
  recognition domain
HDNGNHNINGHDNIHDNININHHDNININHNHHDNI

SEQ ID NO: 50 - protein, 36 aa, Artificial Sequence
  recognition domain
HDNGNHNHHDHDHDHDNGNGHDHDNGNHHDHDHDNG

SEQ ID NO: 51 - protein, 36 aa, Artificial Sequence
  recognition domain
NHNHHDHDHDHDNGNGHDHDNGNHHDHDHDNGHDNG

SEQ ID NO: 52 - protein, 36 aa, Artificial Sequence
  recognition domain
HDNGNGHDNGHDHDHDNHHDNGHDNGHDNHHDNGHD

SEQ ID NO: 53 - protein, 36 aa, Artificial Sequence
  recognition domain
HDNGHDHDHDNHHDNGHDNGHDNHHDNGHDNGHDNG

SEQ ID NO: 54 - protein, 36 aa, Artificial Sequence
  recognition domain
NHHDNINGNHHDNGNINHHDNGNIHDHDNGNGHDNG

SEQ ID NO: 55 - protein, 36 aa, Artificial Sequence
  recognition domain
HDHDHDNHHDNGHDNGHDNHHDNGHDNGHDNGNHHD

SEQ ID NO: 56 - protein, 36 aa, Artificial Sequence
  recognition domain
HDNGHDNHHDNGHDNGHDNGNHHDNINGNHHDNGNI

SEQ ID NO: 57 - protein, 36 aa, Artificial Sequence
  recognition domain
HDNHHDNGHDNGHDNGNHHDNINGNHHDNGNINHHD

SEQ ID NO: 58 - protein, 36 aa, Artificial Sequence
  recognition domain
HDNGHDNGNHHDNINGNHHDNGNINHHDNGNIHDHD

SEQ ID NO: 59 - protein, 36 aa, Artificial Sequence
  recognition domain
HDNGNHHDNINGNHHDNGNINHHDNGNIHDHDNGNG

SEQ ID NO: 60 - protein, 36 aa, Artificial Sequence
  recognition domain
NINGNHNGHDNINIHDNGNGHDNIHDNGNGNHNGHD

SEQ ID NO: 61 - protein, 36 aa, Artificial Sequence
  recognition domain
NHNGHDNINIHDNGNGHDNIHDNGNGNHNGHDNGHD

SEQ ID NO: 62 - protein, 36 aa, Artificial Sequence
  recognition domain
NHHDNGNINHHDNGNIHDHDNGNGHDNGNINHHDNG

SEQ ID NO: 63 - protein, 36 aa, Artificial Sequence
  recognition domain
NINHHDNGNIHDHDNGNGHDNGNINHHDNGNINGHD

SEQ ID NO: 64 - protein, 36 aa, Artificial Sequence
  recognition domain
NIHDHDNGNGHDNGNINHHDNGNINGHDNGNINHHD

SEQ ID NO: 65 - protein, 36 aa, Artificial Sequence
  recognition domain
NHNGHDNGHDNGHDNGHDHDNININININHNINGNI

SEQ ID NO: 66 - protein, 36 aa, Artificial Sequence
  recognition domain
HDNGNINHHDNGNINGHDNGNINHHDHDNGHDNGNI

SEQ ID NO: 67 - protein, 36 aa, Artificial Sequence
  recognition domain
HDNGHDNGHDNGHDHDNININININHNINGNINGHD

SEQ ID NO: 68 - protein, 36 aa, Artificial Sequence
  recognition domain
NINHHDNGNINGHDNGNINHHDHDNGHDNGNINHNH

SEQ ID NO: 69 - protein, 36 aa, Artificial Sequence
  recognition domain
HDNGHDNGHDHDNININININHNINGNINGHDNHNG

SEQ ID NO: 70 - protein, 36 aa, Artificial Sequence
  recognition domain
HDNGHDHDNININININHNINGNINGHDNHNGNING

SEQ ID NO: 71 - protein, 36 aa, Artificial Sequence
  recognition domain
NINGHDNGNINHHDHDNGHDNGNINHNHNGHDHDNI

SEQ ID NO: 72 - protein, 36 aa, Artificial Sequence
  recognition domain
HDHDNININININHNINGNINGHDNHNGNINGHDNI

SEQ ID NO: 73 - protein, 36 aa, Artificial Sequence
  recognition domain
HDNGNINHHDHDNGHDNGNINHNHNGHDHDNINING

SEQ ID NO: 74 - protein, 36 aa, Artificial Sequence
  recognition domain
NINHHDHDNGHDNGNINHNHNGHDHDNININGNHHD

SEQ ID NO: 75 - protein, 36 aa, Artificial Sequence
  recognition domain
HDNGNINHNHNGHDHDNININGNHHDNIHDNGHDHD

SEQ ID NO: 76 - protein, 36 aa, Artificial Sequence
  recognition domain
NINGHDNHNGNINGHDNIHDHDHDNINGNHNHNHHD

SEQ ID NO: 77 - protein, 36 aa, Artificial Sequence
  recognition domain
NINHNHNGHDHDNININGNHHDNIHDNGHDHDHDNG

SEQ ID NO: 78 - protein, 36 aa, Artificial Sequence
  recognition domain
HDNHNGNINGHDNIHDHDHDNINGNHNHNHHDNINI

SEQ ID NO: 79 - protein, 36 aa, Artificial Sequence
  recognition domain
HDHDNININGNHHDNIHDNGHDHDHDNGHDHDNGNG

SEQ ID NO: 80 - protein, 36 aa, Artificial Sequence
  recognition domain
HDNGNGNHHDHDNINGNINHNIHDHDNHNHNIHDNI

SEQ ID NO: 81 - protein, 36 aa, Artificial Sequence
  recognition domain
NHHDNIHDNGHDHDHDNGHDHDNGNGNINGNININI

SEQ ID NO: 82 - protein, 36 aa, Artificial Sequence
  recognition domain
HDHDHDNGHDHDNGNGNINGNININIHDNININHNH

SEQ ID NO: 83 - protein, 36 aa, Artificial Sequence
  recognition domain
HDHDNGNGNINGNININIHDNININHNHNINIHDHD

SEQ ID NO: 84 - protein, 36 aa, Artificial Sequence
  recognition domain
NHNHHDHDNINGNHNIHDHDHDHDHDHDNGHDHDHD

SEQ ID NO: 85 - protein, 36 aa, Artificial Sequence
  recognition domain
NINGNININIHDNININHNHNINIHDHDHDNGHDHD

SEQ ID NO: 86 - protein, 36 aa, Artificial Sequence
  recognition domain
HDHDHDNINHHDHDHDHDNINIHDHDNGNINGNING

SEQ ID NO: 87 - protein, 36 aa, Artificial Sequence
  recognition domain
HDHDNGNGHDNHHDHDNGHDNGHDNGNGNHHDHDNI

SEQ ID NO: 88 - protein, 36 aa, Artificial Sequence
  recognition domain
HDNHHDHDNGHDNGHDNGNGNHHDHDNINGNINHNI

SEQ ID NO: 89 - protein, 36 aa, Artificial Sequence
  recognition domain
HDNGHDNGNGNHHDHDNINGNINHNIHDHDNHNHNI

SEQ ID NO: 90 - protein, 36 aa, Artificial Sequence
  recognition domain
NINGNINGHDNIHDHDNGNINHHDNHHDNINHHDNG

SEQ ID NO: 91 - protein, 36 aa, Artificial Sequence
  recognition domain
NINGHDNIHDHDNGNINHHDNHHDNINHHDNGNIHD

SEQ ID NO: 92 - protein, 36 aa, Artificial Sequence
  recognition domain
NINHHDNHHDNINHHDNGNIHDNHHDNGHDNGHDNG

SEQ ID NO: 93 - protein, 36 aa, Artificial Sequence
  recognition domain
NIHDNHHDNGHDNGHDNGNGHDNGHDHDHDNHHDNG

SEQ ID NO: 94 - protein, 36 aa, Artificial Sequence
  recognition domain
HDNGHDNGNGHDNGHDHDHDNHHDNGHDNGHDNHHD

SEQ ID NO: 95 - DNA, 303 bp, Zea mays
cctctttatccttaaataagaagcataaaacgggatttctcagccagttcttaacttctcttataaatacagaccttgtacaacactttcacctcctctcaggtggccaggatattttttctggccccttcctgccctcttcacactgacccatgtgttgttctatccctggaaagctatacctccttacccctatcagcttctcctcacatctcctctcgtcgccacccatgctatcaccgctctgatcacaagcaaggcaaaccctcactgttctatcaacgcccctcccttagctagatg

SEQ ID NO: 96 - DNA, 303 bp, Zea mays
gcaagagccagcccccggccgtatgtcaacttcacttgtctctctccaaaagatatcgtatcacccatgggcaatggccatgacccccctcccagccccaacctatatcacctagcgcagctacgctctcttctcccgctctcgctctctgcatgctagctaccttctagctatctagcctctaggtccaatgcactccctccttataaacaaggaaccctccttcgcctctcttgccatagaccggacaccggagagctaggtcacaggagcgctcaggaaggccgctgagatagaggcatg

SEQ ID NO: 97 - DNA, 23 bp, Zea mays
caccgctctgatcacaagcaagg

SEQ ID NO: 98 - DNA, 23 bp, Zea mays
cccatgtgttgttctatccctgg

SEQ ID NO: 99 - DNA, 23 bp, Zea mays
acacatgggtcagtgtgaagagg

SEQ ID NO: 100 - DNA, 27 bp, Zea mays
atcaccgctctgatcacaagcaaggca

SEQ ID NO: 101 - DNA, 28 bp, Zea mays
tccctggaaagctatacctccttacccc

SEQ ID NO: 102 - DNA, 28 bp, Zea mays
ttcctgccctcttcacactgacccatgt

SEQ ID NO: 103 - DNA, 23 bp, Zea mays
gtctatggcaagagaggcgaagg

SEQ ID NO: 104 - DNA, 23 bp, Zea mays
tttataaggagggagtgcattgg

SEQ ID NO: 105 - DNA, 23 bp, Zea mays
tagcatgcagagagcgagagcgg

SEQ ID NO: 106 - DNA, 28 bp, Zea mays
tatggcaagagaggcgaaggagggttcc

SEQ ID NO: 107 - DNA, 28 bp, Zea mays
tttataaggagggagtgcattggaccta

SEQ ID NO: 108 - DNA, 28 bp, Zea mays
tcccgctctcgctctctgcatgctagct

SEQ ID NO: 109 - DNA, 19 bp, Zea mays
tcctgccctcttcacactg

SEQ ID NO: 110 - DNA, 19 bp, Zea mays
tctttatccttaaataaga

SEQ ID NO: 111 - DNA, 19 bp, Zea mays
tgccctcttcacactgacc

SEQ ID NO: 112 - DNA, 19 bp, Zea mays
tatccttaaataagaagca

SEQ ID NO: 113 - DNA, 19 bp, Zea mays
tccttaaataagaagcata

SEQ ID NO: 114 - DNA, 19 bp, Zea mays
tcttcacactgacccatgt

SEQ ID NO: 115 - DNA, 19 bp, Zea mays
tgttctatcaacgcccctc

SEQ ID NO: 116 - DNA, 19 bp, Zea mays
tctatcaacgcccctccct

SEQ ID NO: 117 - DNA, 19 bp, Zea mays
tatcaacgcccctccctta

SEQ ID NO: 118 - DNA, 19 bp, Zea mays
tgttgttctatccctggaa

SEQ ID NO: 119 - DNA, 19 bp, Zea mays
tgttctatccctggaaagc

SEQ ID NO: 120 - protein, 19 aa, Zea mays
TCTATCCCTGGAAAGCTAT

SEQ ID NO: 121 - DNA, 19 bp, Zea mays
tatccctggaaagctatac

SEQ ID NO: 122 - DNA, 19 bp, Zea mays
tctcagccagttcttaact

SEQ ID NO: 123 - DNA, 19 bp, Zea mays
tggaaagctatacctcctt

SEQ ID NO: 124 - DNA, 19 bp, Zea mays
tatacctccttacccctat

SEQ ID NO: 125 - protein, 19 aa, Zea mays
TACCTCCTTACCCCTATCA

SEQ ID NO: 126 - DNA, 19 bp, Zea mays
tccttacccctatcagctt

SEQ ID NO: 127 - DNA, 19 bp, Zea mays
tctcttataaatacagacc

SEQ ID NO: 128 - DNA, 19 bp, Zea mays
tcttataaatacagacctt

SEQ ID NO: 129 - DNA, 19 bp, Zea mays
tacccctatcagcttctcc

SEQ ID NO: 130 - DNA, 19 bp, Zea mays
tataaatacagaccttgta

SEQ ID NO: 131 - DNA, 19 bp, Zea mays
tatcagcttctcctcacat

SEQ ID NO: 132 - DNA, 19 bp, Zea mays
tacagaccttgtacaacac

SEQ ID NO: 133 - DNA, 19 bp, Zea mays
tctcctcacatctcctctc

SEQ ID NO: 134 - DNA, 19 bp, Zea mays
tcctcacatctcctctcgt

SEQ ID NO: 135 - DNA, 19 bp, Zea mays
tgtacaacactttcacctc

SEQ ID NO: 136 - DNA, 19 bp, Zea mays
tacaacactttcacctcct

SEQ ID NO: 137 - DNA, 19 bp, Zea mays
tctcctctcgtcgccaccc

SEQ ID NO: 138 - DNA, 19 bp, Zea mays
tcctctcgtcgccacccat

SEQ ID NO: 139 - DNA, 19 bp, Zea mays
tctcgtcgccacccatgct

SEQ ID NO: 140 - DNA, 19 bp, Zea mays
tcgtcgccacccatgctat

SEQ ID NO: 141 - DNA, 19 bp, Zea mays
tcgccacccatgctatcac

SEQ ID NO: 142 - DNA, 19 bp, Zea mays
tccctggaaagctatacct

SEQ ID NO: 143 - DNA, 19 bp, Zea mays
tgctatcaccgctctgatc

SEQ ID NO: 144 - DNA, 19 bp, Zea mays
tatcaccgctctgatcaca

SEQ ID NO: 145 - DNA, 19 bp, Zea mays
tctgatcacaagcaaggca

SEQ ID NO: 146 - DNA, 19 bp, Zea mays
tctggccccttcctgccct

SEQ ID NO: 147 - DNA, 19 bp, Zea mays
tggccccttcctgccctct

SEQ ID NO: 148 - DNA, 19 bp, Zea mays
tcttctcccgctctcgctc

SEQ ID NO: 149 - DNA, 19 bp, Zea mays
tctcccgctctcgctctct

SEQ ID NO: 150 - DNA, 19 bp, Zea mays
tgcatgctagctaccttct

SEQ ID NO: 151 - DNA, 19 bp, Zea mays
tcccgctctcgctctctgc

SEQ ID NO: 152 - DNA, 19 bp, Zea mays
tctcgctctctgcatgcta

SEQ ID NO: 153 - DNA, 19 bp, Zea mays
tcgctctctgcatgctagc

SEQ ID NO: 154 - DNA, 19 bp, Zea mays
tctctgcatgctagctacc

SEQ ID NO: 155 - DNA, 19 bp, Zea mays
tctgcatgctagctacctt

SEQ ID NO: 156 - DNA, 19 bp, Zea mays
tatgtcaacttcacttgtc

SEQ ID NO: 157 - DNA, 19 bp, Zea mays
tgtcaacttcacttgtctc

SEQ ID NO: 158 - DNA, 19 bp, Zea mays
tgctagctaccttctagct

SEQ ID NO: 159 - DNA, 19 bp, Zea mays
tagctaccttctagctatc

SEQ ID NO: 160 - DNA, 19 bp, Zea mays
taccttctagctatctagc

SEQ ID NO: 161 - DNA, 19 bp, Zea mays
tgtctctctccaaaagata

SEQ ID NO: 162 - DNA, 19 bp, Zea mays
tctagctatctagcctcta

SEQ ID NO: 163 - DNA, 19 bp, Zea mays
tctctctccaaaagatatc

SEQ ID NO: 164 - DNA, 19 bp, Zea mays
tagctatctagcctctagg

SEQ ID NO: 165 - DNA, 19 bp, Zea mays
tctctccaaaagatatcgt

SEQ ID NO: 166 - DNA, 19 bp, Zea mays
tctccaaaagatatcgtat

SEQ ID NO: 167 - DNA, 19 bp, Zea mays
tatctagcctctaggtcca

SEQ ID NO: 168 - DNA, 19 bp, Zea mays
tccaaaagatatcgtatca

SEQ ID NO: 169 - DNA, 19 bp, Zea mays
tctagcctctaggtccaat

SEQ ID NO: 170 - DNA, 19 bp, Zea mays
tagcctctaggtccaatgc

SEQ ID NO: 171 - DNA, 19 bp, Zea mays
tctaggtccaatgcactcc

SEQ ID NO: 172 - DNA, 19 bp, Zea mays
tatcgtatcacccatgggc

SEQ ID NO: 173 - DNA, 19 bp, Zea mays
taggtccaatgcactccct

SEQ ID NO: 174 - DNA, 19 bp, Zea mays
tcgtatcacccatgggcaa

SEQ ID NO: 175 - DNA, 19 bp, Zea mays
tccaatgcactccctcctt

SEQ ID NO: 176 - DNA, 19 bp, Zea mays
tcttgccatagaccggaca

SEQ ID NO: 177 - DNA, 19 bp, Zea mays
tgcactccctccttataaa

SEQ ID NO: 178 - DNA, 19 bp, Zea mays
tccctccttataaacaagg

SEQ ID NO: 179 - DNA, 19 bp, Zea mays
tccttataaacaaggaacc

SEQ ID NO: 180 - DNA, 19 bp, Zea mays
tggccatgacccccctccc

SEQ ID NO: 181 - DNA, 19 bp, Zea mays
tataaacaaggaaccctcc

SEQ ID NO: 182 - DNA, 19 bp, Zea mays
tcccagccccaacctatat

SEQ ID NO: 183 - DNA, 19 bp, Zea mays
tccttcgcctctcttgcca

SEQ ID NO: 184 - DNA, 19 bp, Zea mays
tcgcctctcttgccataga

SEQ ID NO: 185 - DNA, 19 bp, Zea mays
tctcttgccatagaccgga

SEQ ID NO: 186 - DNA, 19 bp, Zea mays
tatatcacctagcgcagct

SEQ ID NO: 187 - DNA, 19 bp, Zea mays
tatcacctagcgcagctac

SEQ ID NO: 188 - DNA, 19 bp, Zea mays
tagcgcagctacgctctct

SEQ ID NO: 189 - DNA, 19 bp, Zea mays
tacgctctcttctcccgct

SEQ ID NO: 190 - DNA, 19 bp, Zea mays
tgggcaatggccatgaccc

SEQ ID NO: 191 - DNA, 19 bp, Artificial Sequence
  primer
ggtacagctggtgatggta

SEQ ID NO: 192 - DNA, 18 bp, Artificial Sequence
  primer
gactcttcttcctccctt

SEQ ID NO: 193 - DNA, 20 bp, Artificial Sequence
  primer
cgtctcccccttcaggatgt

SEQ ID NO: 194 - DNA, 21 bp, Artificial Sequence
  primer
gtccaacagggacagttccaa

SEQ ID NO: 195 - DNA, 13 bp, Artificial Sequence
  primer
accaccaatcttg

SEQ ID NO: 196 - DNA, 24 bp, Artificial Sequence
  primer
caggatgctgaaggagctctacta

SEQ ID NO: 197 - DNA, 24 bp, Artificial Sequence
  primer
tggaaccagtagaagacgttcttg

SEQ ID NO: 198 - DNA, 15 bp, Artificial Sequence
  primer
atccggtcgcccagc

SEQ ID NO: 199 - DNA, 978 bp, Artificial Sequence
  cDNA of Zea mays WUS2 protein (wus2)
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccttcaggcgcggcgcctccctcgcccaccctcggcttctacgccgccggcaatggcggcggatcggctgggctgctggacacgagttccgactggggcagcagcggcgctgccatggccaccgagacatgcttcctgcaggactacatgggcgtgacggacacgggcagctcgtcgcagtggccatgcttctcgtcgtcggacacgataatggcggcggcggcggccgcggcgcgggtggcgacgacgcgggcgcccgagacactccctctcttcccgacctgcggcgacgacgacgacgacgacagccagcccccgccgcggccgcggcacgcagtcccagtcccggcaggcgagaccatccgcggcggcggcggcagcagcagcagctacttgccgttctggggtgccggtgccgcgtccacaactgccggcgccacttcttccgttgcgatccagcagcaacaccagctgcaggagcagtacagcttttacagcaacagcacccagctggccggcaccggcagccaagacgtatcggcttcagcggccgccctggagctgagcctcagctcatggtgctccccttaccctgctgcagggagcatgtga

SEQ ID NO: 200 - DNA, 879 bp, Artificial Sequence
  cDNA of Arabidopsis thaliana Homeodomain-like superfamily protein (WUS)
atggagccgccacagcatcagcatcatcatcatcaagccgaccaagaaagcggcaacaacaacaacaacaagtccggctctggtggttacacgtgtcgccagaccagcacgaggtggacaccgacgacggagcaaatcaaaatcctcaaagaactttactacaacaatgcaatccggtcaccaacagccgatcagatccagaagatcactgcaaggctgagacagttcggaaagattgagggcaagaacgtcttttactggttccagaaccataaggctcgtgagcgtcagaagaagagattcaacggaacaaacatgaccacaccatcttcatcacccaactcggttatgatggcggctaacgatcattatcatcctctacttcaccatcatcacggtgttcccatgcagagacctgctaattccgtcaacgttaaacttaaccaagaccatcatctctatcatcataacaagccatatcccagcttcaataacgggaatttaaatcatgcaagctcaggtactgaatgtggtgttgttaatgcttctaatggctacatgagtagccatgtctatggatctatggaacaagactgttctatgaattacaacaacgtaggtggaggatgggcaaacatggatcatcattactcatctgcaccttacaacttcttcgatagagcaaagcctctgtttggtctagaaggtcatcaagaagaagaagaatgtggtggcgatgcttatctggaacatcgacgtacgcttcctctcttccctatgcacggtgaagatcacatcaacggtggtagtggtgccatctggaagtatggccaatcggaagttcgcccttgcgcttctcttgagctacgtctgaactag

SEQ ID NO: 201 - DNA, 795 bp, Artificial Sequence
  cDNA of Triticum aestivum cultivar Avalon WUSCHEL-Like-B1 (WUSCHELL-B1) gene
atggcggcgacggcgactgcgacggcggcggcgacgagcgtggtgacggggacgacgcggtggtgcccgacgccggagcagctgatgatcctggaggagatgtaccgcggcgggctgcgcacccccaacgcgtcgcagatccagcagatcacggcgcacctggcccactacggccgcatcgagggcaagaacgtcttctactggttccagaaccacaaggcccgggaccgccagaagctccgccgcaggctctgcatgagccaccacctcctctcctgcgcccactactacgccgccgccaacgccggccagtaccaccaccagcagcagctcctcggcgccggcgcggttccccctccgctgctgcagcaccagcagcagcagcagtactactccgcctcctgcgccggcggcagctacgaccagcacctgctcccgacgaccgtcccagcttccgcttatgctgctgctgctgctgggtacgcctaccccttcgccgccgtgccggcgagccggtgcgccgacccctcgccgcccaacacgccgctgtccttccatcaccagggtggaggcgtagtaggatcgccggagtactcactggggaggctgggcaacttcggcgtggtggacgacacgtgccggccgtcgcggtgcgagcagcagccacagcagctggccgtggcgacggaagatcaggcggcgccggtgacggcgacggggctgttctgccggccgctgaagacgctggacctcttccccggcgcgatcaaggaggagcagcgcgatgtcgcctag

SEQ ID NO: 202 - DNA, 795 bp, Artificial Sequence; identical to SEQ ID NO: 201
  cDNA of Triticum aestivum cultivar Cadenza WUSCHEL-Like-B1 (WUSCHELL-B1) gene

SEQ ID NO: 203 - DNA, 795 bp, Artificial Sequence; identical to SEQ ID NO: 201
  cDNA of Triticum aestivum cultivar Badger WUSCHEL-Like-B1 (WUSCHELL-B1) gene

SEQ ID NO: 204 - DNA, 795 bp, Artificial Sequence
  cDNA of Triticum aestivum cultivar Charger WUSCHEL-Like-B1 (WUSCHELL-B1) gene
atggcggcgacggcgactgcgacggcggcggcgacgagcgtggtgacggggacgacgcggtggtgcccgacgccggagcagctgatgatcctggaggagatgtaccgcggcgggctgcgcacccccaacgcgtcgcagatccagcagatcacggcgcacctggcccactacggccgcatcgagggcaagaacgtcttctactggttccagaaccacaaggcccgggaccgccagaagctccgccgcaggctctgcatgagccaccacctcctctcctgcgcccactactacgccgccgccaacgccggccagtaccaccaccagcagcagctcctcggcgccggcgcggttccccctccgctgctgcagcaccagcagcagcagcagtactactccgcctcctgcgccggcggcagctacgaccagcacctgctcccgacgaccgtcccagcttccgcttatgctgctgctgctgctgggtacgcctaccccttcgccgccgtgccggcgagccggtgcgccgacccctcgccgcccaacacgccgctgtccttccatcaccagggtggaggcgtagtaggatcgccggagtactcactggggaggctgggcaacttcggcgtggtggacgacacgtgccggccgtcgcggtacgagcagcagccacagcagctggccgtggcgacggaagatcaggcggcgccggtgacggcgacggggctgttctgccggccgctgaagacgctggacctcttccccggcgcgatcaaggaggagcagcgcgatgtcgcctag

SEQ ID NO: 205 - DNA, 795 bp, Artificial Sequence; identical to SEQ ID NO: 204
  cDNA of Triticum aestivum cultivar Claire WUSCHEL-Like-B1 (WUSCHELL-B1) gene

SEQ ID NO: 206 - DNA, 795 bp, Artificial Sequence; identical to SEQ ID NO: 204
  cDNA of Triticum aestivum cultivar Spark WUSCHEL-Like-B1 (WUSCHELL-B1) gene

SEQ ID NO: 207 - DNA, 2130 bp, Artificial Sequence
  cDNA of Zea mays AP2-like ethylene-responsive transcription factor BBM2 (LOC103650883)
atggccactgtgaacaactggctcgctttctccctctccccgcaggagctgccgccctcccagacgacggactccacgctcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaagtccaactgcaacttgatacccagcactagcagcacagtttgctacgcgagctcagctgctagcaccggctaccatcaccagctgtaccagcccaccagctccgcgctccacttcgcggactccgtcatggtggcctcctcggccggtgtccacgacggcggttccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaaggacaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgtctcaaggaggccgaggccgcagcgtccgcgcagcaccaccacgccggcgtggtgagctacgacgtcggccgcatcgcctcgcagctcggcgacggcggagccctagcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacttgaacctgggcgcggccggcgcgcacgactttttctcggcagggcagcaggccgccgccgcagctgcgatgcacggcctggctagcatcgacagtgcgtcgctcgagcacagcaccggctccaactccgtcgtctacaacggcggggtcggcgatagcaacggcgccagcgccgttggcagcggcggtggctacatgatgccgatgagcgctgccggagcaaccactacatcggcaatggtgagccacgagcagatgcatgcacgggcctacgacgaagccaagcaggctgctcagatggggtacgagagctacctggtgaacgcggagaacaatggtggcggaaggatgtctgcatgggggaccgtcgtctctgcagccgcggcggcagcagcaagcagcaacgacaacattgccgccgacgtcggccatggcggcgcgcagctcttcagtgtctggaacgacacttaa

SEQ ID NO: 208 - DNA, 1707 bp, Artificial Sequence
  cDNA of Arabidopsis thaliana Integrase-type DNA-binding superfamily protein (PLT2)
atgaattctaacaactggctcgcgttccctctatcaccaactcactcttctttgccgcctcacattcactcttcacaaaattctcatttcaatctaggtttggtcaacgacaatatcgacaacccttttcaaaaccaaggatggaatatgatcaatccacatggtggaggcggcgaaggtggagaggttccaaaagtggctgatttcttaggagtgagcaaatcgggggatcatcacaccgatcacaacctcgtaccttataacgacattcatcaaaccaacgcctccgactactactttcaaaccaatagcttgttacctacagtcgtcacttgtgcctctaatgctcctaataattatgagcttcaagagagtgcacacaatttgcaatctctcactctctctatgggaagtactggagctgccgctgcagaagtcgccactgtgaaagcctcgccggctgagactagtgccgataatagtagcagcactaccaacacaagtggaggagccatcgttgaggctacaccgagacggactttggaaacttttggacaacgaacctctatctatcgtggagttacaagacatagatggaccggtagatatgaagctcatctttgggataatagctgtagaagagaaggacaatcaaggaaaggaagacaagtctacttaggtgggtatgacaaagaagagaaagcagccagagcatatgatctagctgcacttaaatattggggtccctctactactaccaactttccgataactaactacgagaaggaagtagaggagatgaaaaacatgacgagacaagagtttgtggcttctataagaaggaaaagtagcggattctcgcgtggtgcatccatgtatcgtggagtaacaaggcatcatcaacatggaagatggcaagcaaggatcggccgagttgctggaaacaaagatctctacttgggaacattcagcacggaggaagaagcagcagaagcttatgacatagctgcgataaagtttcgaggtctaaacgcggttacaaactttgagataaatcggtatgatgtgaaagccatcctggagagcaacacacttcctataggaggtggtgcggctaaacggctcaaagaagctcaagctctagaatcatcaagaaaacgagaggaaatgatagccctcggatcaaatttccatcaatatggtgcagcgagcggctcgagctctgttgcttccagctctaggcttcagcttcaaccttaccctctaagcattcaacaaccttttgagcatcttcatcatcatcagcctttacttactctacagaacaacaacgatatctctcagtatcatgattcctttagttacattcagacgcagcttcatcttcaccaacaacaaaccaacaattacttgcagtcttctagtcacacttcacagctctacaatgcttatcttcagagtaaccctggtctgcttcatggatttgtctctgataataacaacacttcagggtttcttggaaacaatgggattggtattgggtcaagctctaccgttggatcatcggctgaggaagagtttccagccgtgaaagtcgattacgatatgcctccttccggtggagctacagggtatggaggatggaatagtggagagtctgctcaaggatcgaatccaggaggtgttttcacgatgtggaatgaataa

SEQ ID NO: 209 - DNA, 1818 bp, Artificial Sequence
  cDNA of Beta vulgaris subsp. vulgaris AP2-like ethylene-responsive transcription factor PLT2 (LOC104889956)
atgggctcaatgaattcaaacaattggttgtcttttcctctttctcctacacatccttcacttcaatcacatcttcaaaccaatgattcacaacctcatcaacaattctccttgggtcttgtatctgaccacattgacaacccctttggtcaagcgcaagaatggaacttgctcaatccacaagggccaaatgaagtacccaaaatagcagatttcttaggagtagggaattcagaaactcatcattcaccagaccttacagcgttcagtgacatgagccaaggtggtgaatcagattatcttttctccggcaacggcggcggcttaatggcggtgcaaaacaccgtagcagcagctactaatagtagccaatatgatcaataccaagagaactctaataattgcttgcaatctttgactctatcaatgggaagtagtggacaacagcctcaacaacagcaacaaccaccttcaagcactaataattgtgagactagtggtgacaataatagcaccgctagtgtcgccgcctctactgccgccactgtcaccaccgcgattactcctgtggttgaagccacccctaggagaaccttggatacttttggccaaaggacttctatttatagaggtgttacaaggcataggtggacaggaagatatgaagctcatctttgggataatagttgtagaagggaaggacagtcaaggaagggtcgtcaagtgtatcttggagggtatgataaggaagagaaggccgctaggtcttatgatttagctgcaatcaagtattggggaacttcaactactacaaattttccaataagcaactatgagaaagaaatagaagacatgaaacacatgactagacaagaatttgtagcagctattagaaggaagagtagtggattctctagaggtgcatcaatttatcgtggtgtaacaagacaccatcaacatgggagatggcaagcaagaattggaagggtggcaggaaacaaggatctctacttaggaacatttagcacagaggaagaggctgcagaagcttatgatatcgcggctatcaagtttagaggccttaatgctgtgacaaattttgacatgagccggtatgatgttaaagccatcctagagagcaacactcttcccataggaggaggggcggcgaagcgccttaaggaagctcaagctatagaatcctctaggaagagggaagaaatgcttgccctaagcaatagtagctacccatatggagctagtagctcgagctcgactcgatatggagcccatcaacaagcaacaactcatgcataccctttgttaccataccaccatcaagaccatcaaccacaacctttgctaaccctacaaaataaccatggtcaagaaagcaatatttccctatcacattactctcaagaggctcaattccttcagttgtaccaacaatcaagttactcaaaccctagtagcatgtacaacaattacctccaaactaaccctagtttgcttcatgggttcatgaacatgggctcaaactcttgtggtgttattgatactaacaatactaatggaagttcaagtgggagttatagtggtggagggtaccttggtggtggggctgggatcaatgccatgggtgccgcctcgacaacgagcaatgcggtggtttccggtgaaccggagccacttgcattggtgaaggtggactatgatatgccttctgctggtggtggtggaggaagttatgaggggtggtcaactgagacggttcaaggacctaataatggggtttttacaatgtggaatgactaa

SEQ ID NO: 210 - DNA, 2157 bp, Artificial Sequence
  cDNA of Beta vulgaris subsp. vulgaris AP2-like ethylene-responsive transcription factor BBM (LOC104890283)
atgggttcaatgaattggttaggtttctctttatctcctcaagaacttccttcacaaactcctgatcatggtagtaatcaagatcaccatcatcatcactttacaagcaacaacaatggagagtgtttcgatctcgggcccggctcaacgcctcattcttctctcaatcacatcccttcttcctttggaatccttgaggccttccatagatcaactaatgatcaatcccaagattggaacaatatgaagggaaactcagagcttagtatgctaatgggaaaccaagaagttgaagaggagccaaaactagaaaactttctagggagtagtcactcttttagagagaatcatcatcaaaataatggagatctctacatgtttaatactacacatgataacaacaataatagtactatgtcaaaccctaaggatattactagtcctgctagtaataataataataataataataacggactcaatgtttcaatgatcaagacatggttgagatcaaaccaccctcctcaatcaaatatagtggatggtggtggtggcagtggtggcggcggggcgaatgcacaaacattatccctttcaatgggaactggtgtgtcccaatccgccttgccgctactagcggcaggaggaggaggtggtggtggtggaggagagatagagagtagtttgtctgagaatagtagtagtaataataaacaacaattaagtgatacaacggccgggatatgtaataacacagctagtactattactgctatcgttgatgttcaaagtagtgcactagaaagcgttcctaggaaatctattgatacatttggacaacgtacatccatttaccgtggtgtaacaagacataggtggactgggagatatgaagctcatctatgggataatagctgtaggagagaagggcagactcgtaagggcagacaagtttatttggggggttatgacaaagaagaaaaagcggctagagcttatgatttggctgcacttaaatattggggtaccactaccaccaccaactttcctattactgattatgaaaaggaagttgaggatatgaagcatatgacacgccaagaatatgtggcatctctacgaaggaaaagtagtggattttctcgtggtgcatcaatttatcgaggagtaacaaggcatcatcagcatggtcgttggcaagcaaggataggtagggttgcaggcaacaaagacctctacctgggaactttcagtacacaagaagaagcagcagaagcatatgatatagcagcaataaagtttaggggattaaatgcagtaacaaactttgagataaacaggtatgatgtgaaagccatacttgatagcaccacacttcctataggaggagcagcaaagaggttaaaagatgtggaggatttaaccacaattactccagataaacagattattagggcaattacttcgagtaatgataataatcatgaaaattctcagcttactaattttggtaatgggactcccaatttccattcctggcctggaatcgcattcccacaagctcaaccacttgcaatgcattacccttatgcaacttctcaacaacaacaacaacaacaacaaaggttttggtgtaagcaagaagttcaagatactactaatgattaccaagatcatcttaatcagcagcttcaaatgaataatgggacacataatttctttcagatgcataatttgatggggttggagaattcttctactagtttggagcatagttctgggtcgaattccgtcgtttatgggaatgggaatgggaatgggaatggaaatgatcatggtgttgggaatgggtatggattaccctttgggatgtcaacagtaattgctcatgatgggaatgggaatggaagtgggaatgggaatgaacaaagtgggtatgagaattattactatctttcacaccaaggaaataataataatcatggtaatgctgctggtgtaagaggagctgttgggacttatgatcaagggtcagcttgtaacaattgggtcccaacggcgattccgacactcgttccgaggccgaataatatggcggctgttggtggtcatggtggaggaggaatccctactttcactgtgtggaatgacacctaa

SEQ ID NO: 211 - DNA, 1884 bp, Artificial Sequence
  cDNA of Triticum aestivum WANT1-2 mRNA for AP2 transcription factor
atgagagcgatggccagcggcggcggcaactggttaggcttctccctctccccgcacatggccatggaggtgccctcctcctctgaacccgaccacgctcagcctgctagcgctagtgctatgtctgcttctcccaccaacgccgcgacctgcaacctcctattctcccaacccgcgcaaatggccgctccacctcctggatactactacgtcggcggcgcctatggggatggcaccagcaccgctggcgtctactactcccaccaccccgtcatgcccatcacgtccgatggatctctgtgcatcatggaagggatgatgccgtcgtcctcgccgaagctcgaggacttcttgggtggcggcaatggcagcggacatgacgcggtcacctactacagccaccagcagcaggaccaacaagaccaggaggcaagcagaatctaccagcaccatcaacagcagcagcagcagctagcgccctacaacttccagcacttgacggaagcagaggcgatctaccaagaggccacggcgccgatggacgaggcaatggccgctgccaagaaccagctggtgacgagctacggctcatgctacagcaacgcggggatgcagccgctgagcctgtccatgagccccaggtcccagtccagcagctgcgtcagcgcagctcctcagcagcatcagatggctgcggctgctgctgctgcctccttggctgcttcccagggaggcagtaatggtggtggggagcaggagcagtgcgtggggaagaagaggggcactgggaagggaggccagaagcagcccgttcatcgcaagtccatcgacacgtttgggcagaggacctcccagtataggggcgtcaccaggcacaggtggactgggagatatgaagcccacctctgggacaacagctgcaagaaggatgggcagacaaggaaagggaggcaagtttatctaggtggttatgacaatgaagacaaggctgccagggcttatgatctggctgctctgaaatattgggggccgtcgacgaacaccaatttcccgctagaaaattatcgagaggaggtcgaggagatgaaaagcatgacaaggcaggaattcgttgcacacttgagaaggagaagcagcgggttttctcgtggtgcttcgatatatcgaggagtaacgaggcatcatcagcatggaagatggcaagctaggattggcagggttgctggcaacaaagacttgtatctcggcactttcaccactcaggaagaagcagccgaggcctacgacgtagccgcgatcaagttccgtggcctgaacgccgtgaccaacttcgacataaccagatacgacgtggacaagatcatggagagcagctctctgctgcccggtgacgaagcgcgcaaggtcaaggcggtcgaggcagccaaccacgtgcctgccatgcacaacggcggcggggagatcagccatgccgaagaaggaagctccggcgtctggaggatggtactccatggaacaccgcagcaagctgcacagtgcacccccgaggtggcagaccttcagaagggcttcatgggcggcggcgaccctcgctcgtccctgcatggcatcgccgggttcgacgtcgagtcggcggcgcatgacatcgacgtctcaggcaagatcaactactccaacccgtcctccctggtgaccagcctcagcaactcgagagaggggagcccagagaggttcagcctgccctcgctgtacgccaagcatcccaacgccgtcagcgtcgccagcatgagcccgtggatggcgatgccagcgccggccgccgcccacgtgttaagggggccgaattcctccatgcctgtgttcgctgcctggacggacgcatag

SEQ ID NO: 212 - DNA, 1896 bp, Artificial Sequence
  cDNA of Triticum aestivum WANT1 mRNA for AP2 transcription factor
atgagagcgatggccagcggcggcggcaactggttaggtttctccctctccccgcacatggccatggaggtgccctcctctgaacccgaccacgctcaggctcaacctgctagcgctagcgctatgtccgcttctcccacaaacgccgcgacctgcaacctcctattctcccaacccgcgcaaatggccgctccacctcctggctactactacgtcggcggcgcctatggggatggcaccagcaccgccggcgtctactactcccaccactccgtcatgcccatcacgtccgatggatccctgtgcatcatggaagggatgatgccatcgtcctcgccgaagctcgaggacttcttgggtggcggcaatggaagtgggcacgacgcggtcacctactacagccaccaccagcagcagcaggaccaacaggaccaggaggcaagcagaatctaccagcaccatcagcagcagctagcgccctacaacttccagcacttgacggaaacggaggcgatctaccaagagaccacggcgccgatggatgaggcaatggccgctgccaagaacctgctcgtgacgagctatggctcatgctacagcaacgcggggatgcagccgctgagcctgtccatgagccccaggtcccagtccagcagctgcgtcaccgcagctcctcagcagcatcagatggctgcggctgctgctgctgctgctgcctctatggctgcttcccagggaggcagtaatggtggtggggagcagtgcgtggggaagaagaggggcactgggaagggaggccagaagcagcccgttcaccgcaagtccatcgacacgtttgggcagaggacctcccagtataggggcgtcaccaggcacaggtggactgggagatatgaagcccacctgtgggacaacagttgcaagaaggatgggcagacaaggaaagggaggcaagtttatctaggtggttatgataatgaagacaaggctgccagggcttatgatctggctgctctgaaatactgggggccgtcgacgaacaccaatttcccgctagaaaattatcgagaggaggtcgaggagatgaaaagcatgacaaggcaggaattcgttgcacacttgagaaggagaagcagcgggttttctcgtggtgcttcgatatatcgaggagtaacgaggcatcatcagcatggaagatggcaagctaggattggcagggttgctggcaacaaagacttgtatctcggcactttcaccactcaagaagaagcagccgaggcctatgacgtagccgcgatcaagttccgtggcctgaacgccgtgaccaacttcgacataaccagatacgacgtggacaagatcatggagagcagctctctgctgcccggggacgaagcgcgcaaggtcaggccgatcgaggcggccaaccacgtgccttccatgcacaacggcggcggggagctcagccatgccgaagaaggaagctcaggcgtctggaggatggtgctccatggaacaccgcagcaagctgcacagtgcacccccgaggtggccgaccttcagaagggcttcatggacggcgaccctcgctcgtccctgcatggcaatggcattgccgggttcgacgtcgagtctgccgcgcatgacatcgacgtttcaggcaagattaactactccaactcgtcttccctggtgaccagcctcagcaactcgagagaggggagccccgagaggttcagcctgccctcgctgtacgccaagcatcccaacgccgtcagcctcgccaccatgagcccgtggatggcgatgccggcgccgaccgccacccacgcgttgagggggccgaattcctccatccctcccatgcctgtgtttgctgcctggacagacgcatag

SEQ ID NO: 213 - DNA, 2382 bp, Artificial Sequence
  cDNA of Triticum aestivum clone tplb0046e23, cultivar Chinese Spring
gggttggcccctccctctcattccttttgctcagctcacgggtccctctcgcccgtcttcctcgtagttcacttctcttttaccaccactgcctccatctccatgtcgtcgctcggacaagggtagtggtgccgcagtagcagtagagctcagctcagagtgaaagcgaagcaagaagcgttttcgtctgtgtttgtttgttgatgagagcgatggccagcggcggcaactggttaggcttctccctctccccgcacatggccatggaggtgccctcctcctctgagcccgaccacgctcagcctgctagcgctagcgctatgtccgcttctcccaccaacgccgccacctgcaacctcctcttctcccctccctcgcaaatggccgctccacctcctggctactactacgtcggcggggcctacggggatggcaccagcaccgccggcgtttactactcccaccaccccgtcatgcccatcacgtccgatggatccctgtgcatcatggaagggatgatgccgtcgtcctcgccgaagctcgaggacttcttgggtggcggcaatggcagtgcgcacgacgcggtcacctactacagccaccaccagcagcagcagcaggaccaacaggaccaggaggtaagcagaatctaccagcaccatcagcagcagctagcgccctacaacttccagcacttgacggaggcagaggcgatctaccaagaggccacggcgccgacggatgaggcaatggccgctgccaagaacctgctcgtgacgagctatggctcatgctacagcaacgcggggatgcagccgctgagcctgtccatgagccccaggtcccagtccagcagctgcgtcagcgcagctcctcagcagcatcagatggctgcggttgctgctgcggctgctgcctctatggttgcttcccagggaggcagtaatggtggtggggagcagtgcgtggggaagaagaggggcactgggaagggaggccagaagcagcccgttcatcgcaagtccatcgacacgtttgggcagaggacctcccagtataggggcgtcaccaggcacaggtggactgggagatatgaagcccacctgtgggacaacagttgcaagaaggatgggcagacaaggaaagggaggcaagtttatctaggtggttatgacaatgaagacaaggctgccagggcttatgatctggctgctctgaaatattgggggccatcgacgaacaccaatttcccgctagaaaattatcgagaggaggtcgaggagatgaaaagcatgacaagacaggaattcgttgcacacttgagaaggagaagcagcgggttttctcgtggtgcttcgatatatcgaggagtaacgaggcatcatcagcatggaagatggcaagctaggattggcagggttgctggcaacaaagacttgtatctcggcactttcaccactcaggaagaagcagctgaggcctacgacgtagcggcgatcaagttccgtggcctgaacgccgtgaccaacttcgacataaccagatacgacgtggacaagatcatggagagcagctctctgctgcccggggacgaagcgcgcaaggtcaggccgatcgaggcagccagccacgtgtctcccatgcacaacggcggcggggagctcagccatgccgaagaaggaagctccggcgtctggaggatggtgctccatggaacaccgcagcaagctgcgccgtgcacccccgaggtggccgaccttcagaagggcttcatggacggcgaccctcgctcgtccctgcatggcaatggcattgccgggttcgacgtggagtctgcggcgcatgacatcgacgtctcaggcaagatcaactactccaactcgtcttccctggtgaccagcctcagcaactcgagagaggggagccccgagaggttcagcctaccctcgctgtacgccaagcatcccaacgccgtcagcctcgccagcatgagcccgtggatggcgatgccggcgccgaccgccgcccacacgttgaggggaccgaattcctccatcccttctatgcctgtgtttgctgcctggacggacgcatagccgtgttgcagctgctcaaatcttgctgtcactggccatgttgtagtaaactggagctggattagtagcgtcgttgctcatgtcgcttaagtttaatctgggaaggctggttaattggttatcacgaaggcggtgtagtggtagtggtagtggtacgtaggagaagcatgcattagtctctagctcaccgaacttgtagcagtacgtagtgttcttacttactttcttttgagcctataacaatgcatggaaggaggctgtcccaagaaaaaaaaaaaaaaaacga

SEQ ID NO: 214 - DNA, 2528 bp, Artificial Sequence
  cDNA of Triticum aestivum clone WT012_J17, cultivar Chinese Spring
gacacacgcgcgcacagaccaaagtcccccttcaaacccgctgagcttgcaatggagagcagcggcatcattgcgacatgtgctccccaatgattgatcctctcattcccatctaagctagatcttcttgaatcttgagaccaccacagcctcatccccagtcgtgctcgtgcgcccttgctcccatccgctccgcccgatgaccaacggcggccacagcatgagcggcgccagcatcgcgagcggtgctggcggctggctgggtttctcgctgtcgcctcacgtcgccatggaggcggcggccggctccggcatcgtcgacgtggccggccaccaccacgcgcagcacggcggggtctactatcaccctgacgcggtcgcctcctcccccatgtccttctacttcggtgggagcgacaatgtcggcgccgcgagcggcgggtactactccgggatctccgcactgcctctcaggtccgacggctccctctgcctcgccgacgcgctccggaggagcgagcagaaacaccacggggcggaggtgtcggcgccgccgaagctcgaggacttcctgggcgcgagtcccgccatggcgctgagcctggacaactcgggctactactacggcggccaaggccatggccatggcgacgcaggaggcggccagcaccagctgccgtacgccatgatgcctggctccggtggccaccacatgtactacgacgcccacgcggcgttgctggacgagcaggctgcagccacgtcggccgcgatggaagcggccggctggatggcgcgtgccggagacgtctacgacgtggacgccggcaacggcgaggacgccatcgtggcgaccggccacgacaaccccggtgggtacgtacacccgctgacgctgtccatgagctccgggtcccagtccagctgcgtcaccatgcagcaggcggctgcacacgcccacgcctacgtcggtgccggcggcgagtgcgtcggccaggcgaccgcggccagcaagaagcgcggcgcgggcgccgggcagaacaagcagccggtcgtgcaccgcaagtgcatcgacaccttcggccagcgcacgtccaagtaccggggcgtcaccaggcataggtggacggggaggtatgaggcgcacctctgggacaacagctgccggaaggaaggccagaccaggaaaggccggcaagtttatcttggtgggtatgacatggaggagaaggcggcgagggcgtatgacctcgcggcgctcaagtactggggcgcgtccacgcacatcaacttcccggtggaggactaccaggaggagctggaggtgatgaagaacatgaccaggcaggagtatgtggctcacctcagaaggaagagcagcgggttctcgcgcggcgcctcggtgtaccggggagtcaccaggcaccaccagcaggggcggtggcaggcgcgcatcggccgcgtctccggcaacaaggacctctacctcggcacattcagcgcggaggcggacgcggcggaggcgtacgacgtggcggcgatcaagttccgcggcctcaacgcggtcaccaacttcgacatcaaccgctacgacgtggacaagatcatggagagcagcacgctcctgcccggcgaccaggtgcggcgcaggaaggacggccccgacgagagcgccgccgtggtggcaagcgcggcggccgccctcgtgcaggccggcagcgccgcggactactggaggcagcctgcggcggtgaccacggaagagcacagccgccaccacctggaccttctgtcgagcgagtccttctccctgctgcgcggcgtggtgtccctggacggcgacgcggctggtgctcaggggcagggcaaccgcatgtcgggcgcgtcgtccctggccacgagcctgagcaactcccgggagcagagcccggaccagggaggcggcctggccatgctgttcgcccggcccgaggcgccgaagctggcgagctcgctgcccatgggcacctgggtctcatcgccggcgccggccaggcccggtgtgtccgtggcgcacatgccagtgttcgccgcgtgggccgacgcctgacttgctcgactacagcgtcgtccttttggccctgcatccacgaggagatagcaaggttgtttaactaggactggttacctagcattagtagctgcgttagcaaggaactgtaaggtggttttattagccatagctggtagcttagcggcgcatgcatgcatctgcctgggctctcgtggttccttccccagctgcgtctgggacgaagggtttttgtagtatcgagccatggcacggcagcagcagcgtcgcctccggcccggcggagagccgccgccgctgatcggagctggatgggtagctgtagctcctgtctctagacctcctaactttcatcaaaccaaaatgttggaccttcgtgttcgtgtggcctcgcggcgcgtctgaacatctgatttttttattttttttgagggtaagcaaaaaaaaaaaaaaacga

SEQ ID NO: 215 - DNA, 1803 bp, Artificial Sequence
  cDNA of Triticum aestivum PARG-2D
atgaccaacaacaacggcaatggcaatggcggcagcaacgcggcggcgagtggctggctgggcttctcgctctcgccgcacatggacgaacacaaccacgtgcagcagcagcaacagcaccagggcctattctaccccagctccgtcgccgccgcctacagcctcggcggcgacgtcgccaccgacgggtactattcgcagctagcctccatgcctctcaagtcagacggctccctctgcatcatggaagctctacgccgaaccgatcaacaagatcaccacggtccgaagctggaggactttctgggcgcggggcaaccggcgatggcgctgagcctggacaacacctccaacttctattactacggcggcggtggcggagccggtgggcaacacggacagagccacggcggcagcttcctgcagcaagcatacgacgtgtacagcgggcccgcaacggcatcggtgctggcggccaatgaggacgccgcggcagccacggccatggcgaactgggtgcaggtcgcgcgcggtgccaccgcgtacgccacagccgagaacgtcttgtccgcggcggcggaccggcagcagcatcttcaccaccaccctctggcactctccatgagctccgccgggtcgctctccagctgcgttaccgcgggggccgagtacggcggcgtcggggcgacggtggacggcgggcgaaagcgcggcggcgcgacggcggggcagaagcagccggtgcaccaccgcaagtccatcgacacgttcgggcagcgcacgtcgcagtaccgtggcgtcaccaggcataggtggacggggcggtatgaggcgcacctgtgggacaacagctgcaagaaggaaggccagaccaggaaagggaggcaagtttacctcggaggatatgacatggaggagaaggcggcgagagcctacgaccaggcggcgctcaagtactggggcccttccacccatatcaacttcccgctcgaggactaccagcaggagctggaggagatgaagaacatgacgaggcaggagtacgtggcacaccttagaaggaagagcagcggcttctcgcgtggcgcgtccatgtaccgtggcgtgacccggcaccaccagcacgggcggtggcaggcgcgcatcggccgcgtctccggcaacaaggacctctacctcggcactttcggcacccaggaggaggccgcggaggcgtacgacatcgccgccatcaagttccggggcctcaacgccgtcaccaacttcgacatcacccgctacgacgtcgacaagatcatggccagcaacacgctcctcccgggcgagcacgccaggcgcaacaaggacgacaacgccgcgcccctgcccctccccgcccccgacgactgcgccgcctctgccctggtgcccgtgtccactccggggacggacaccggcggcagcggccagcaccgctaccacgacgtcatgtcctcgggcgaggccttctcggcgctacacgacctggtcaccgtggacggccacaccgcgcagggcgggaacggcgcgcacgtgcacatgtcgatgtcgggcgcatcgtcgctggtgacgagcctgagcaactcccgagaggagagcccagaccggggcggcgggctgtccatgctcttcgccaagccgccgcagcagccggccacgacaacggcggcgtccccgaagctgatgagcactctgaagccgctgggctcctgggcgtcgtcggcgaggccggccgccgtttccatcgctcacatgcccatgttcgccgcgtggagcgacgcatga

SEQ ID NO: 216 - DNA, 1806 bp, Artificial Sequence
  cDNA of Triticum aestivum PARG-2A
atgaccaacaacaacggcaatgggaatggcggcagcaacgcggcggcgagtggctggctgggcttctcgctctcgccgcacatggacgaacacaaccacgtgcagcagcagcagcaacaacaccagggcctattctaccccagctccgtcgccgccgcctacagcctcggcagcgacgtcgccaccggcgggtactattcgcagctagcctccatgcctctcaagtcagacggctccctctgcatcatggaagctctacgccgaaccgatcaacaagatcaccacggtccgaagctggaggactttctgggcgcggggcaaccggcgatggcgctgagcctggacaacacctccaacttctattactacagcggcggtggcggagcaggtgggcaacacggacagagccacggcggcggcttcctgcagcaagcatacgacgtgtacggcgggcccgcaacggcatcggtgctggcggccgatgaggacgccgcggcagccacggccatggcgaactgggtgcaggtcgcgcgcggtgccaccgcgtacgccacagccgagaacgtcttgtccgcggcggcggaccggcagcagcatcttcaccaccaccctctggcactctccatgagctccgccgggtcgctctccagctgcgttaccgcgggggccgagtacggcggcgtcgtggcgacggtggacggcgggcgaaaacgcggtggcgcgacggcggggcagaagcagccggtgcaccaccgcaagtccatcgacacgttcgggcagcgcacgtcgcagcaccgtggcgtcaccaggcataggtggacggggcggtatgaggcgcacctgtgggacaacagctgcaagaaggaaggccagaccaggaaagggaggcaagtttacctcggagggtatgacatggaggagaaggcggcgagagcctacgaccaggcggcgctcaagtactgggggccttccacccatatcaacttcccgctcgaggactaccagcaggagctggaggagatgaagaacatgacgaggcaggagtacgtggcacaccttagaaggaagagcagcggcttctcgcgtggcgcgtccatgtaccgtggcgtgacccggcaccaccagcacgggcggtggcaggcgcgcatcggccgcgtctccggcaacaaggacctctatctcggcactttcggcacccaggaggaggccgcggaggcgtacgacatcgccgccatcaagttccggggactcaacgccgtcaccaacttcgacatcacccgctacgacgtcgacaagatcatggccagcaacacgctcctcccgggcgagctcgccaggcgcaacaaggacgccaacgccgcgcccctgcccctccccgcccccgacgactgcgccgcctctgccctggtgcccgtgtctactccggggacggacaccggcggcagcggccagcaccgaaaccaggacgtcatgtcctcgggcgaggccttctcggcgctgcacgacctggtcaccgtggacggccacaccgcgcagggcggcaacggcgcgcgcgtgcacatgtcgatgtcgggcgcatcgtcgctggtgacgagcctgagcaactcccgcgaggagagcccagaccggggcggtggcctgtctatgctcttcgccaagccgccgcagcagccggccacgacaacggcggcgtccccgaagctgatgagcactctggcgccgctgggttcctgggcgtcgtcggcgaggccggccgccgtttccatcgctcacatgcccatgttcgccgcgtggagcgacgcatga

SEQ ID NO: 217 - DNA, 2040 bp, Artificial Sequence
  cDNA of Zea mays BBM
atggcttcagcgaacaactggctgggcttctcgctctcgggccaggataacccgcagcctaaccaggatagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctctccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtgcccagcggcgccgccagcggagcggcctcggagagcacatcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagggaagggcagagtcgcaagggtaggcaagtttaccttggtggctatgacaaggaggacaaggcagcaagggcttatgatttggcagctctcaagtattggggcactacgacaacaacaaatttccctataagcaactacgaaaaggagctagaagaaatgaaacatatgactagacaggagtacattgcatacctaagaagaaatagcagtggattttctcgtggggcgtcaaagtatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggtgcggccaggcgcctcaaggacgccgtggaccacgtggaggccggcgccaccatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcgcacagcctgcaggacctccagcagctgcacctcggcagcgcggcccacaacttcttccaggcgtcgtcgagctccacagtctacaacggcggcgccggcgccagtggtgggtaccagggcctcggtggtggcagctctttcctcatgccgtcgagcactgtcgtggcggcggccgaccaggggcacagcagcacggccaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtga

SEQ ID NO: 218 - DNA, 963 bp, Artificial Sequence
  cDNA of Zea mays WUS1
atggcggccaacgtgggcgcgggcaggagtgctggcggcggcggagccggcactggcactggcactgctgctggcagcggcggcgtgtcgacggccgtgtgccgccctagcggctcgcggtggacgccgacgccggagcagatcaggatcctcaaggagctctactacggctgcggcatccggtcgcccaactcggagcagatccagcgcatcaccgccatgctgcggcagcacggcaagatcgagggcaagaacgtcttctactggttccagaaccacaaggcccgcgagcgccagaagcgccgcctcaccaacctcgacgtcaacgtgcccgtcgccgccgacgacagcgcccaccgccttggcgtcctctcgttgtcgccttcttcaggttgttcaggcgcggcgcctccgtcgcccaccctcggcttctacgccggcggcaatggctccgctgtgatgctggacacgagttccgattggggcagcgctgctgccatggccactgaggcatgcttcatgcaggactacatgggcgtgatgggcggcgcgtcaccgtgggcatgctcctcctcgtcgtcggaggacccgatggcggcgctggcgctggcgccgaaggtgacccgggcgcccgagacgctccctctcttcccgaccggcggcggcggagacgataggcagcccccgcggccgcggcagtctgtcccagcaggcgaggccatccgcggcggcagcagcagcagcagctaccttccgttctggggtgccgcgcccaccccaactggcagtgccacttccgttgcgatccagcagcaacaccagctgatgcagatgcaagagcagtacagcttttacagcaacgcccagctgctgcccggcaccggcagccaggatgcagcagcaacatccctggagctgagcctcagctcctggtgctccccttaccctgcagggaccatgtga

SEQ ID NO: 219 - DNA, 978 bp, Artificial Sequence; identical to SEQ ID NO: 199
  cDNA of Zea mays WUS2

SEQ ID NO: 220 - DNA, 975 bp, Artificial Sequence
  cDNA of Zea mays WOX2
atggagacgccacagcagcaatccgccgccgccgccgccgccgccgcccacgggcaggacgacggcgggtcgccgccgatgtcgccggcctccgccgcggcggcggcgctggcgaacgcgcggtggaacccgaccaaggagcaggtggccgtgctggaggggctgtacgagcacggcctgcgcacccccagcgcggagcagatacagcagatcacgggcaggctgcgggagcacggcgccatcgagggcaagaacgtcttctactggttccagaaccacaaggcccgccagcgccagaggcagaagcaggacagcttcgcctacttcagcaggctcctccgccggcccccgccgctgcccgtgctctccatgccccccgcgccaccgtaccatcacgcccgcgtcccggcgccgcccgcgataccgatgccgatggcgccgccgccgcccgctgcatgcaacgacaacggcggcgcgcgtgtgatctacaggaacccattctacgtggctgcgccgcaggcgccccctgcaaatgccgcctactactacccacagccacagcagcagcagcagcagcaggtgacagtcatgtaccagtacccgagaatggaggtagccggccaggacaagatgatgaccagggccgcggcgcaccagcagcagcagcacaacggcgccgggcaacaaccgggacgcgccggccaccccagccgcgagacgctccagctgttcccgctccagcccaccttcgtgctgcggcacgacaaggggcgcgccgccaacggcagtaataacgactccctgacgtcgacgtcgacggcgactgcgacagcgacagcgacagcgacagcgtccgcttccatctccgaggactcggatggcctggagagcggcagctccggcaagggcgtcgaggaggcgcccgcgctgccgttctatgacttcttcgggctccagtcctccggaggccgctga

SEQ ID NO: 221 - DNA, 666 bp, Artificial Sequence
  cDNA of Zea mays WOX5
atggaggcgctgagcgggcgggtaggcgtcaagtgcgggcggtggaaccctacggcggagcaggtgaaggtcctgacggagctcttccgcgcggggctgcggacgcccagcacggagcagatccagcgcatctccacccacctcagcgccttcggcaaggtggagagcaagaacgtcttctactggttccagaaccacaaggcccgcgagcgccaccaccacaagaagcgacgccgcggcgcgtcgtcgtcctcccccgacagcggcagcggcaggggaagcaacaacgaggaagacggccgtggtgccgcctcgcagtcgcacgacgccgacgccgacgccgacctcgtgctgcaaccgccagagagcaagcgggaggccagaagctatggccaccatcaccggctcgtgacatgctacgtcagggacgtggtggagcagcaggaggcgtcgccgtcgtgggagcggccgacgagggaggtggagacgctagagctcttccccctcaagtcgtacggcgacctcgaggcggcggagaaggtccggtcgtacgtcagaggaagcggcgccaccagcgagcagtgcagggagttgtccttcttcgacgtcgtctccgccggccgggatccgccgctcgagctcaggctctgcagcttcggtccctag

SEQ ID NO: 222 - DNA, 1521 bp, Artificial Sequence
  cDNA of Zea Mays WOX8
atggcgtcctcgaacaggcactggccgagcatgtacaggtccagtctcgcctgcaacttccagcagccgcagccgcagcctgacatgaacaacggcggcaagtcctcactcatgtcctcaaggtgcgaggagaacggcggaaggaacccggagccgaggccgcggtggaacccgcggccggagcagatcaggatcctggaagggatcttcaactccggcatggtgaacccgccgcgcgacgagatccgccgcatccgcctccaactgcaggagtacgggcccgtcggcgacgccaacgtcttctactggttccagaaccgcaagtcccgcaccaagcacaagctgcgcgccgcggggcagctgcagccgtcgggctcgggccgctccgccctgcaggcgcgcgcgtgcgccccggcgcccgtgacgcctcccaggaacctgcagctcgcggccgctgctcccgtggcgccgcccacgtcctcgtcctcgtcgtcctccgaccggtcctcggggtcatcatcgagcaagtcggtgaccgtgaccccgacgaccgccgtcgcgcttgcttctcccgcaggcgccgcgccggctgctgtcttccgccagcagggcgtgatgccgacgacggccatggacctgcttacgccgctgccgtcgtcgtcggccgctctggccgcgcgccagctctactatcagtaccacagccagatcatggcgcctgccgcgccgccgatgcccgatacggtgatcgcctctccggagcagttccttccgcagtggcagcagggcggacagcagcattattacctgccggccaccgagctcggtggcgtcctcgacggccactcccaccacacacacgagcccccggcggccatacaccggcccgtctcgctctcacccagcgtgctctttggcctgtgcaacgaagctctaaggcaagactactgcgccgacatcagcgtcgtccccaccaagggactcggccatggccaccagttctggaacagcaccacctgcggctctgatatgggcaatagcaatagcaagatcgacgccgtgagcgccgtgatcagggacgacgagaagtccaggctggggttactccactactacggcttggcgggcgcgacgacgaccgctgctgcggctgtcgctccggcccctctcgctgcagatgccgccgccggtacggccacgctgcttccaagctctgcggcgagcgaccagttgcaagggctgttggatgctgctgggctgctgatgggggagacgccgccgacgccgacggcgacggtggtggccgtggcccgggacgccgtgacgtgcgcggccaccgccaccgcgcagttcagcgtgccggcgtcgatgcgcctggacgtgaggctggcgttcggcgaggccgcccttctggcgcgccacaccggcgaggcggtccccgtcgacgagtccggcgtcacggtggagccgctccagcaggacactctctactacgtgctcatgcaggcgactaataactga

SEQ ID NO: 223 - DNA, 822 bp, Artificial Sequence
  cDNA of Zea mays WOX10
atggagtgggtggacaggaccaaggcctccgccgccgccgccgcagcggcggcggacgagagggctgggggagcggaagggctcgcgggatacgtcaaggtcatgaccgacgaacagatggaggtgctccgcaagcagatctccatctacgccaccatctgcgagcagcttgtcgagatgcaccgcgccctcaccgagcaccaggacaccattgcaggaattaggtttagtaatctgtactgtgatcctcaaattatccctggaggccacaagatcacagcaaggcaacgatggcaaccaacaccaatgcagctgcagatcttggagaacatctttgaccaaggcaatggaacaccaagcaagcagaggataaaggagataacggcagagctctcgcaccatggccaaatctcggagacaaatgtgtacaactggttccagaacagacgggcacggtcaaagcggaagcaggccgcttctttaccgaacaatgctgaatctgaagctgaggtggacgaggagtctctcaccgataagaagccgaagtcagatcggtcgctccaggacaacaaggctatgggcgctcacaacgctgacaggatatctgggatgcatcacttggacactgatcatgaccaaatcggtggcatgatgtatggatgcaatgacaacggcttgagatcgtctggcagttctggccagatgtccttctacgggaacatcatgccgaatccaagaatcgatcatttcccggggaaggtggagagctcccggagcttctcccatctccaacacggggaaggctttgacatgtttggatga

SEQ ID NO: 224 - DNA, 849 bp, Artificial Sequence
  cDNA of Zea mays WOX13
atggactgggggaacaggaccaaggccgccgccgccgctgcggcgccggacgagagggccgggggaggggaagggctcggaggatacgtcaaggtcatgaccgacgaacagatggaggtgctccgcaagcagatctccatctacgccaccatctgcgagcagcttgtcgagatgcatcgcgtcctcaccgagcaccaggacaccattgcaggattgaggtttagcaatctgtactgtgaccctctaatcatccccggcggtcacaagatcacggcaaggcagcggtggcaaccaacaccgatgcagctgcagatcctggagagcatcttcgaccagggcaacgggacaccgagcaagcagaagataaaggagataacagcggagctctcgcagcacggccagatctcggagacgaacgtgtacaactggttccagaacaggcgggcacggtcgaagcggaagcaggccgctgcttccttaccgaacaacgccgaatccgaagccgaggcggacgaggagcctctcgccgacaagaagccgaagtcagacaggccgccgccgccgccgccgccgatccaggataataccaaggctacgggcgctctcagcgccgacagggtctctggtgggacgcgtcacttggacacgggtcatgaccagaccagtggcgtgatgtatgggtgcaacgacagtggcttgttgagatcgtccggcagttcgggccagatgtccttgtacgagaacttcatgtcgaatccaagaatcgatcgtttcccggcgaaggtggagagctcccggagcttcccccatctccaacaacacggggaaggctttggcatgtttggatga

SEQ ID NO: 225 - DNA, 795 bp, Artificial Sequence
  cDNA of Zea mays Lec1
atggactccagcttcctccctgccggcgcggacaatggctcggcgggcggcgccaacaatggcggcggcgctgctcagcaggcgccgccgatccgcgagcaggaccggctgatgccgatcgcgaacgtcatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcagcgtctacctccaccgctaccgcgagttcgagggcgaggcgcggggcgtcggcctcgccccggcccctccgcgcggcgaccaccaccaccaccaccactccgtgccgccatcgatgctcaacaagtcccgcgggcccggctccggagccgtcatgctaccgcaccaccaccaccacgacatgcacgcctccatgtacgggggcgccgtgcccccgccgccgcaccacggcttcctcatgccacacccacagggcggccactacctgccttacccctacgagcccacgtcgtacggcggcgagcacgccttggccagcgggtactatggaggggccgcgtacgcgccgggcaacaacggcgggagcggcgatggcagcggcgggagcgcgtcgcacgcaccgccgggcggcagcggcggcggcttcgaccacccgcacacgttcgcgtacaagtag

SEQ ID NO: 226 - DNA, 1179 bp, Artificial Sequence
  cDNA od Zea mays Lec2
atgccagcccgcgcctcccacccggcgcttgccacctcgcgcgcgcgcggttggccgcgcctgcgcgccctcggcatcgcccccgacggggggcgttggcgttgcctcccccactttgcacccatttcagagcccgcccgacacttgtcaccgcgcgcccccgcctccgcgtctccgcccgcccgcccccatccggctataaaagcctcgccctctccaaccctagccgccgctgccgctgccgccgccgccgctacctcctcccttccttccttctccgctcgtcgtcgttctaccggcatggccggcattaccaagcgccgcacctccccggcctccacctcctcttcgtccggcgacgtcttgccgcagcgggtcacccggaagcgtcggtccgcccgccgcgggccccggagcaccgcccgtaggccgtcggcgcctccacctatgaatgaactggacttgaatacagctgctcttgatccggatcattatgctacaggattgagagttcttcttcagaaggagctccgaaatagcgatgtaagccagcttgggagaattgttctcccaaagaaggaggcggagtcttacctccctattctgatggcaaaggatggaaagagtttatgcatgcatgacttgctaaattcacaactgtggaccttcaagtatagatattggttcaacaacaaaagcaggatgtatgtgcttgaaaataccggagattatgtaaaagctcatgaccttcagcaaggagacttcatcgtgatctacaaggacgacgagaacaaccgctttgtcataggagcaaagaaggcaggagatgagcagaccgccactgtacctcaagtccatgaacacatgcacatctctgccgcactgccagctccacaagcgttccatgactatgcaggccccgtcgcagcagaagctggtatgctcgcgatcgtgccacagggtgacgagatattcgacggcatactgaactccctgccggagataccagtggcgaacgtgaggtactccgacttcttcgacccgttcggtgactccatggacatggcgaatccgctgagctcctccaataacccctcggtcaacctggctacgcatttccatgacgagaggatcgggagctgctcgtttccctacccaaaatccgggcctcagatgtga

SEQ ID NO: 227 - DNA, 1026 bp, Artificial Sequence
  cDNA of Zea mays WIND1_1
atggccgcagccatcgacatgtacaagtactacaataccagcgcacaccagatcccctcctcatccccctcggatcaggagctcgcgaaagcactcgagccttttataacgagtgcttcctcctcttcatcctcctccccctaccatggctactcgtcctctccatccatgtcccaagattcttacatgcctacaccctcttacaccagctacgccacctcgcctcttcccactcccgccgccgcctcctcctcgcagcttccgccgctctactcgtcgccttatgcggcgccgtgcatggccggccagatgggcctgaaccagctcggcccggcccagatccagcagatccaggcccagttcatgttccagcagcagcagcagcagcagaggggcctgcacgcggcgttcctgggcccgcgggcgcagccgatgaagcagtcagggtcgccgtcgccgccgccgccgctggcgccggcgcagtcgaagctgtaccgcggcgtgcggcagcgccactggggcaagtgggtggcggagatccggctcccgaagaaccgcacgcggctgtggctcggcaccttcgacaccgcggaggacgcggcgctcgcctacgacaaggcggccttccgcctccgcggcgacacggcgcgcctcaacttcccggccctccggcgcggcggcgcgcacctcgccggcccgctgcacgcctccgtggacgccaagctgaccgccatctgccagtccctgtcggagtccaagtccaagagcggctcgtccggcgacgagtcggccgcgtccccgccggactcccccaagtgctcggcgtcgacgacggagggagagggggaggaggagtcgggctccgccggctcccctcctcctcctcctcctcccccgacgctggcgccgcccgtgccggagatggcgaagctggacttcacggaggcgccgtgggacgagacggaggccttccacctgcgcaagtacccgtcctgggagatcgactgggattccatcctgtcatga

SEQ ID NO: 228 - DNA, 951 bp, Artificial Sequence
  cDNA of Zea mays WIND1_2
atggccgcagccatagacatgtacaagtactgcaataccagcgcacaccttatcgcctcctcgtccccctcggatcaggagctcgcgaaagcactcgagccttttataacgagtgcttcctccccctaccatcgctactcgttggccccagattcttacatgcctacaccctcctcctacaccacctcgcctcttcccacccccacctcctcgcctttctcgcagcttccgccactctactcgtcgccttacgcggcttcgacggcgtcgggcgtggctgggccgatgggcctgaaccagctcggcccggcccagatccagcagatccaggcccagctcatgttccagcaccagcagcagaggggcctgcacgcggcgttcctgggcccgcgggcgcagccgatgaagcagtccgggtcgccgccggcgcagtcgaagctgtaccgcggcgtgcgccagcgccactggggcaagtgggtggcggagatccgcctccccaagaaccgcacgcggctgtggctcggcaccttcgacaccgccgagggcgcggcgctggcctacgacgaggcggccttccgcctccgcggcgacacggcgcgcctcaacttcccgtccctccgccgcggcggcggcgcgcgcctcgccggcccgctccacgcctccgtggacgccaagctcaccgccatctgccagtccctggcggggtccaagaacagctcgtccagcgacgagtcggccgcgtccctgccggactcccccaagtgctcagcgtcgacggagggggatgaggactcggcctccgccggctcccctccttccccgacgcaggcgccgcccgtgccggagatggcgaagctggacttcaccgaggcgccgtgggacgaaacggaggccttccacctgcgcaagtacccgtcctgggagatcgactgggattccatcctctcatga

SEQ ID NO: 229 - DNA, 702 bp, Artificial Sequence
  cDNA of Zea mays ESR1_1
atggcgccgagaacgtcagagaaaaccatggcaccggcggcggccgctgccacggggctcgcgctcagcgtcggcggcggcggcggggccggcggcccgcactacagaggcgtgaggaagcggccgtggggccggtacgcggcggagatccgcgacccggcgaagaagagccgggtgtggctcggcacctacgacacggccgaggacgccgcgcgggcctacgacgccgccgcgcgcgagtaccgcggcgccaaggccaagaccaacttcccttacccctcgtgcgtgcccctctccgcagccggttgccggagcagcaacagcagcaccgtcgagtccttcagcagcgacgcgcaggcgcccatgcaggccatgccgctcccgccgtcgctcgagctggacctgttccaccgcgcggcggccgcggccacgggcacgggcgctgccgccgtacgcttccctttcggcagcatccccgttacgcacccgtactacttcttcgggcaggccgcagccgcagccgcggaagcagggtgccgtgtgctcaagctggcgccggcggtcaccgtggcgcagagcgactccgactgttcgtcggtagtggatctgtcgccgtcgccaccggccgctgtgtcggcgaggaagcccgccgcgttcgatctcgacctgaactgctcaccgccgacggaggcggaagcctag

SEQ ID NO: 230 - DNA, 885 bp, Artificial Sequence
  cDNA of Zea mays ESR1_2
atggaggacgtggccaacgcacacatctacgcccacgcccaccggagcaagcgtccccagtcggccgcgatcaaagacggggacggggacgtcgacctgtccatgaaaggcgcgcggtaccgcggcgtgcggcgccggccgtggggccggttcgcggcagagatccgcgaccccatgtccaaggagcggcggtggctcggcaccttcgacaccgccgagcaggccgcctgcgcctacgacatcgcggcgcgcgccatgcgcggcaacaaggcgcgcaccaacttcccgggccacgccacggcgggctactggccgtggggcgcgccgcagccggcggcggtggcgcacccgatcaaccctttcctcctgcacaacctcatcatgagctcctccaaccacggctgccgcctgctcaaccacgcaggccacggacacgtccactccgcagcccccagacctccggcgccggcggcggacgccacgtccacgaccatcgcagcgcccttccctgtcgccgcacaccccgccgtagcgatggacgaggacgtggacgactgggacggcgtcctgcggagcgagcccgcggacgccgggctgctgcaggacgcgctgcacgacttctaccctttcacgcgtccgcgcgccggcgggggcaggcgcggcctgtccgcggccggaaccgacgccagggcggcagctgcgttggtggcgccggtaaagccggatgctttcgtcgttcccagccctttcgccggcgtcgagggggacggtgaatacccgatgatgccgcagggcctgctcgaggacgtgatccactccccggcgttcgtggaggttgtggccgcgccgccgtccgtccccacgcgccgcggccgccggggctga

SEQ ID NO: 231 - DNA, 2130 bp, Artificial Sequence; identical to SEQ ID NO: 207
  cDNA of Zea mays PLT3

SEQ ID NO: 232 - DNA, 1479 bp, Artificial Sequence
  cDNA of Zea mays PLT5
atggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctaccgaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcggcggcggcggcggcgggtggccggagcagcaaggcgctggagtcccctccgtccggctcgcttgacggcggcggcggcatgccagtcgtcgaagccagcacggcaccgccgctgttcattccggtgaagtacgaccagcagcagcaggagtacctgtcgatgctcgcgttgcagcagcaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcctccgcctcgcatgagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatga

SEQ ID NO: 233 - DNA, 1467 bp, Artificial Sequence
  cDNA of Zea mays PLT7
atggacatggacatgagctcagcttatccccaccattggctctccttctccctctccaacaactaccaccatggcctactcgaagccttctctaactcctccggtactcctcttggagacgagcagggcgcagtggaggagtccccgaggacggtggaggacttcctcggcggcgtcggtggcgccggcgccccgccgcagccggcggcggctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggccggggcggagtccgaccaggcgaggcggcccgccgagacgttcggccagcgcacatccatctaccgtggcgtcaccaggcaccggtggacggggagatatgaggcgcacctgtgggacaacagctgccgccgggagggccaaagccgcaaaggacggcaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaactttgacatgagccgctacgacgtggagagcatcctcagcagcgacctccccgtcgggggcggagctagcggtcgcgcccccgccaagttcccgttggactcgctgcagccggggagcgctgccgccatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctatggtttctggatctgctggctacgagggctccaccggcgacaatggaacctgggttactacgactaccagcagcaacaccggcacggctccccactactacaactatctcttcgggatggagtag

SEQ ID NO: 234 - DNA, 1413 bp, Artificial Sequence
  cDNA of Zea mays IPT
atggcccacccctccgccgccgccgccgccgtatcctccacggcgcccgctgcaaaccctagttctggcgcccgcgaggaaggaggcgcccgctctccgccgtcgccgtctccgtctcagagggggcgggccaaggtggtgatcgttatgggcgccacgggcgccggcaagtcgcggctggccgtcgacctcgcggcccacttcgccggcgtcgaagtggtcagcgccgactccatgcagctctaccgcggcctcgacgtcctcaccaacaaggctcccctccacgagcagaacggtgttcctcatcatctacttagcgtgattgatccctctgtcgagttcacttgccgtgatttccgcgaccgtgccgtgccgattatacaggaaatagtggaccgcggtggcctccctgtggttgtcggcggcacaaacttctacatccaggctctcgttagcccattcctcttggatgatatggcagaagaaatgcagggctgtactctgagagatcacatagatgatggtcttactgatgaagatgaaggcaatgggtttgaacgcttgaaggagatcgatcctgtggctgcgcagaggatccatccaaacgaccatagaaaaatcaaacgctacctcgagttgtatgcaaccacgggtgccctacccagcgatctgttccaaggagaggccgctaagaaatggggtcggcctagtaactccagactcgactgctgtttcctgtgggtagatgctgatcttcaagtcctggacagttatgtcaacaaaagggtcgattgcatgatggatggtggcctgctggacgaagtatgcagcatatatgatgcggatgctgtctatacccaggggctgcggcaggctattggggttcgtgagtttgacgagtttttcagagcatatttacccagaaaagaatctggtgagggttcctgtgcaagcctgttaggtatgcatgacgatcagcttaagagcttgttggacgaagctgtttcccagctgaaggcaaacactcgtagactagttcgacgtcaaagacggagattgcatcggctgagtaaagattttgggtggaacttgcatcgtgttgacgcaaccgaagcattcttctgtgccactgacgactcatggcaaaagaaagttgtcaaaccatgtgtggatgtcgtaagaaggtttttgtcggacaattccactgttttgccaagcacaagcgcaagtgacccctcttcaagagagctgtggacgcaatatgtgtgcgaggcctgcggcaaccgggtgctgcgaggtgcgcacgagtgggagcagcacaggcaagggcgaggccaccggaagcgagtgcagcgcctgaagcagaagagcctgaggccatggccatcgctgctgccccaagaccgcagctga

SEQ ID NO: 235 - DNA, 1080 bp, Artificial Sequence
  cDNA of Zea mays Knotted1
atggaggagatcacccaacactttggagttggcgcaagcagccacggccatggccacggccagcaccaccatcatcaccaccaccaccacccgtgggcatcctccctcagcgccgtcgtagcgccgctgccgccgcaaccgccaagcgcaggcctgccgctgaccctgaacacggtggcggccactgggaacagcggcggtagcggcaacccggtgctgcagcttgccaacggtggcggcctcctcgacgcatgcgtcaaggcgaaggagccctcgtcgtcgtctccctacgcaggcgacgtcgaggccatcaaggccaagatcatctcgcacccacactactactcgctcctcactgcctacctcgagtgcaacaaggtgggggcaccaccggaggtgtcggcgaggctgacggagatagcgcaggaggtggaggcgcggcagcgcacggcgctcggcggcctggccgctgcgacggagccggagctggaccagttcatggaggcgtaccacgagatgctggtgaagttcagggaggagctgacgaggccgctgcaggaggcgatggagttcatgcgaagggtggagtcgcagctgaactcgctttccatctccggaaggtcgctgcgcaacatcctttcatctggctcttctgaggaggatcaagaaggtagcggaggagagaccgagctccctgaagttgatgcacatggtgtggaccaagagctgaagcaccatctcctgaagaaatacagtggctatctaagctcgctcaagcaagaactgtcaaagaagaagaagaaagggaagctccccaaggaggctcgccagcagctccttagctggtgggatcagcactacaaatggccttacccctcagagactcagaaggtggcactggctgagtctaccgggcttgacctgaagcagatcaacaactggttcatcaaccagcggaagcggcactggaagccatccgaggagatgcaccacctgatgatggacgggtaccacaccaccaatgccttctacatggacggccacttcatcaacgacggcgggctgtaccggctcggctag

SEQ ID NO: 236 - DNA, 936 bp, Artificial Sequence
  cDNA of Zea mays RKD4_1
atgacgggcctcgacgaggcgctcatgctgccgttcaccgacatcgatcttgaggccttcgacaacgccgaagagcaaaagcctcctgtcgaccaaatggttatgatgccgccgacggttgaacaccccgccgccgccgggacgcgagccccaatcatcattgatggtacggcgaccgttggccaaaatgtaggtggtggtgtcgtccacgctcatcagaaggcggccatgacgaccatagaggactccagctgcttccgacgaggagccagctgtgtcgacgacgacatggccgtcgtcattcaccatgtcgagcgtcgtcgtcaagcaggctctaccgccgtggcgctattgccgccgccgcagccgtcactgccgcggccgcgtgcaagggcgagcggcggcgcgggcgagcggtcagctccggcggccgccgggaagacgaggatggaccacatcggcttcgacgagctgcgcaagtacttctacatgcccatcaccagggcggccagggagatgaacgtggggctcaccgtgctcaagaagcgctgccgcgagctcggcgtggcgcggtggcctcaccggaagatgaagagcctcaagtccctcatggccaacgtacaggaaatggggaacggcatgtcgccggtggctgtgcagcatgagcttgcggcgctggagacgtactgcgcgctcatggaggagaacccatggatcgagctcacggaccggacgaagaggctgcggcaggcctgcttcaaggagagctacaagcggaggaaggcggccgcaggcaacgctatcgagacggatcacattgtctacagctttggacagcatcgtcgttacaagcagcagctgctgcctccgccaactgcgggtagtaccagtgctgacgaccgccatggccagagcagccgtttcttttgctactga

SEQ ID NO: 237 - DNA, 1176 bp, Artificial Sequence
  cDNA of Zea mays RKD4_2
atggcgatggtgccatgtggcggtgacgacgcggaatggtgcaatatgatggaggccatcaaccacctgatgatgtcttccatgtcctcgccgcacgtcgccatgggcgccagcagttgcagggaagaggacgacgacagtttgtacttgcccatgtactactcatctgcgccaccgccagccgtcgtcagcgatcagtactgccccgaacaactcccaccgctgcctgctgccggtgcaatgacgggcctcgacgaggcgctcatgctgccgttcaccgacatcgatcttgaggccttcgacaacgccgaagagcaaaagcctcctgtcgaccaaatggttatgatgccgccgacggttgaacaccccgccgccgccgggacgcgagccccaatcatcattgatggtacggcgaccgttggccaaaatgtaggtggtggtgtcgtccacgctcatcagaaggcggccatgacgaccatagaggactccagctgcttccgacgaggagccagctgtgtcgacgacgacatggccgtcgtcattcaccatgtcgagcgtcgtcgtcaagcaggctctaccgccgtggcgctattgccgccgccgcagccgtcactgccgcggccgcgtgcaagggcgagcggcggcgcgggcgagcggtcagctccggcggccgccgggaagacgaggatggaccacatcggcttcgacgagctgcgcaagtacttctacatgcccatcaccagggcggccagggagatgaacgtggggctcaccgtgctcaagaagcgctgccgcgagctcggcgtggcgcggtggcctcaccggaagatgaagagcctcaagtccctcatggccaacgtacaggaaatggggaacggcatgtcgccggtggctgtgcagcatgagcttgcggcgctggagacgtactgcgcgctcatggaggagaacccatggatcgagctcacggaccggacgaagaggctgcggcaggcctgcttcaaggagagctacaagcggaggaaggcggccgcaggcaacgctatcgagacggatcacattgtctacagctttggacagcatcgtcgttacaagcagcagctgctgcctccgccaactgcgggtagtaccagtgctgacgaccgccatggccagagcagccgtttcttttgctactga

SEQ ID NO: 238 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVPSGAASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 239 - protein, 320 aa, Zea mays
MAANVGAGRSAGGGGAGTGTGTAAGSGGVSTAVCRPSGSRWTPTPEQIRILKELYYGCGIRSPNSEQIQRITAMLRQHGKIEGKNVFYWFQNHKARERQKRRLTNLDVNVPVAADDSAHRLGVLSLSPSSGCSGAAPPSPTLGFYAGGNGSAVMLDTSSDWGSAAAMATEACFMQDYMGVMGGASPWACSSSSSEDPMAALALAPKVTRAPETLPLFPTGGGGDDRQPPRPRQSVPAGEAIRGGSSSSSYLPFWGAAPTPTGSATSVAIQQQHQLMQMQEQYSFYSNAQLLPGTGSQDAAATSLELSLSSWCSPYPAGTM

SEQ ID NO: 240 - protein, 325 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPSGAAPPSPTLGFYAAGNGGGSAGLLDTSSDWGSSGAAMATETCFLQDYMGVTDTGSSSQWPCFSSSDTIMAAAAAAARVATTRAPETLPLFPTCGDDDDDDSQPPPRPRHAVPVPAGETIRGGGGSSSSYLPFWGAGAASTTAGATSSVAIQQQHQLQEQYSFYSNSTQLAGTGSQDVSASAAALELSLSSWCSPYPAAGSM

SEQ ID NO: 241 - protein, 324 aa, Zea mays
METPQQQSAAAAAAAAHGQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHARVPAPPAIPMPMAPPPPAACNDNGGARVIYRNPFYVAAPQAPPANAAYYYPQPQQQQQQQVTVMYQYPRMEVAGQDKMMTRAAAHQQQQHNGAGQQPGRAGHPSRETLQLFPLQPTFVLRHDKGRAANGSNNDSLTSTSTATATATATATASASISEDSDGLESGSSGKGVEEAPALPFYDFFGLQSSGGR

SEQ ID NO: 242 - protein, 221 aa, Zea mays
MEALSGRVGVKCGRWNPTAEQVKVLTELFRAGLRTPSTEQIQRISTHLSAFGKVESKNVFYWFQNHKARERHHHKKRRRGASSSSPDSGSGRGSNNEEDGRGAASQSHDADADADLVLQPPESKREARSYGHHHRLVTCYVRDVVEQQEASPSWERPTREVETLELFPLKSYGDLEAAEKVRSYVRGSGATSEQCRELSFFDVVSAGRDPPLELRLCSFGP

SEQ ID NO: 243 - protein, 506 aa, Zea mays
MASSNRHWPSMYRSSLACNFQQPQPQPDMNNGGKSSLMSSRCEENGGRNPEPRPRWNPRPEQIRILEGIFNSGMVNPPRDEIRRIRLQLQEYGPVGDANVFYWFQNRKSRTKHKLRAAGQLQPSGSGRSALQARACAPAPVTPPRNLQLAAAAPVAPPTSSSSSSSDRSSGSSSSKSVTVTPTTAVALASPAGAAPAAVFRQQGVMPTTAMDLLTPLPSSSAALAARQLYYQYHSQIMAPAAPPMPDTVIASPEQFLPQWQQGGQQHYYLPATELGGVLDGHSHHTHEPPAAIHRPVSLSPSVLFGLCNEALRQDYCADISVVPTKGLGHGHQFWNSTTCGSDMGNSNSKIDAVSAVIRDDEKSRLGLLHYYGLAGATTTAAAAVAPAPLAADAAAGTATLLPSSAASDQLQGLLDAAGLLMGETPPTPTATVVAVARDAVTCAATATAQFSVPASMRLDVRLAFGEAALLARHTGEAVPVDESGVTVEPLQQDTLYYVLMQATNN

SEQ ID NO: 244 - protein, 273 aa, Zea mays
MEWVDRTKASAAAAAAAADERAGGAEGLAGYVKVMTDEQMEVLRKQISIYATICEQLVEMHRALTEHQDTIAGIRFSNLYCDPQIIPGGHKITARQRWQPTPMQLQILENIFDQGNGTPSKQRIKEITAELSHHGQISETNVYNWFQNRRARSKRKQAASLPNNAESEAEVDEESLTDKKPKSDRSLQDNKAMGAHNADRISGMHHLDTDHDQIGGMMYGCNDNGLRSSGSSGQMSFYGNIMPNPRIDHFPGKVESSRSFSHLQHGEGFDMFG

SEQ ID NO: 245 - protein, 282 aa, Zea mays
MDWGNRTKAAAAAAAPDERAGGGEGLGGYVKVMTDEQMEVLRKQISIYATICEQLVEMHRVLTEHQDTIAGLRFSNLYCDPLIIPGGHKITARQRWQPTPMQLQILESIFDQGNGTPSKQKIKEITAELSQHGQISETNVYNWFQNRRARSKRKQAAASLPNNAESEAEADEEPLADKKPKSDRPPPPPPPIQDNTKATGALSADRVSGGTRHLDTGHDQTSGVMYGCNDSGLLRSSGSSGQMSLYENFMSNPRIDRFPAKVESSRSFPHLQQHGEGFGMFG

SEQ ID NO: 246 - protein, 264 aa, Zea mays
MDSSFLPAGADNGSAGGANNGGGAAQQAPPIREQDRLMPIANVIRIMRRVLPAHAKISDDAKETIQECVSEYISFITGEANERCQREQRKTITAEDVLWAMSRLGFDDYVEPLSVYLHRYREFEGEARGVGLAPAPPRGDHHHHHHSVPPSMLNKSRGPGSGAVMLPHHHHHDMHASMYGGAVPPPPHHGFLMPHPQGGHYLPYPYEPTSYGGEHALASGYYGGAAYAPGNNGGSGDGSGGSASHAPPGGSGGGFDHPHTFAYK

SEQ ID NO: 247 - protein, 392 aa, Zea mays
MPARASHPALATSRARGWPRLRALGIAPDGGRWRCLPHFAPISEPARHLSPRAPASASPPARPHPAIKASPSPTLAAAAAAAAAATSSLPSFSARRRSTGMAGITKRRTSPASTSSSSGDVLPQRVTRKRRSARRGPRSTARRPSAPPPMNELDLNTAALDPDHYATGLRVLLQKELRNSDVSQLGRIVLPKKEAESYLPILMAKDGKSLCMHDLLNSQLWTFKYRYWFNNKSRMYVLENTGDYVKAHDLQQGDFIVIYKDDENNRFVIGAKKAGDEQTATVPQVHEHMHISAALPAPQAFHDYAGPVAAEAGMLAIVPQGDEIFDGILNSLPEIPVANVRYSDFFDPFGDSMDMANPLSSSNNPSVNLATHFHDERIGSCSFPYPKSGPQM

SEQ ID NO: 248 - protein, 341 aa, Zea mays
MAAAIDMYKYYNTSAHQIPSSSPSDQELAKALEPFITSASSSSSSSPYHGYSSSPSMSQDSYMPTPSYTSYATSPLPTPAAASSSQLPPLYSSPYAAPCMAGQMGLNQLGPAQIQQIQAQFMFQQQQQQQRGLHAAFLGPRAQPMKQSGSPSPPPPLAPAQSKLYRGVRQRHWGKWVAEIRLPKNRTRLWLGTFDTAEDAALAYDKAAFRLRGDTARLNFPALRRGGAHLAGPLHASVDAKLTAICQSLSESKSKSGSSGDESAASPPDSPKCSASTTEGEGEEESGSAGSPPPPPPPPTLAPPVPEMAKLDFTEAPWDETEAFHLRKYPSWEIDWDSILS

SEQ ID NO: 249 - protein, 316 aa, Zea mays
MAAAIDMYKYCNTSAHLIASSSPSDQELAKALEPFITSASSPYHRYSLAPDSYMPTPSSYTTSPLPTPTSSPFSQLPPLYSSPYAASTASGVAGPMGLNQLGPAQIQQIQAQLMFQHQQQRGLHAAFLGPRAQPMKQSGSPPAQSKLYRGVRQRHWGKWVAEIRLPKNRTRLWLGTFDTAEGAALAYDEAAFRLRGDTARLNFPSLRRGGGARLAGPLHASVDAKLTAICQSLAGSKNSSSSDESAASLPDSPKCSASTEGDEDSASAGSPPSPTQAPPVPEMAKLDFTEAPWDETEAFHLRKYPSWEIDWDSILS

SEQ ID NO: 250 - protein, 233 aa, Zea mays
MAPRTSEKTMAPAAAAATGLALSVGGGGGAGGPHYRGVRKRPWGRYAAEIRDPAKKSRVWLGTYDTAEDAARAYDAAAREYRGAKAKTNFPYPSCVPLSAAGCRSSNSSTVESFSSDAQAPMQAMPLPPSLELDLFHRAAAAATGTGAAAVRFPFGSIPVTHPYYFFGQAAAAAAEAGCRVLKLAPAVTVAQSDSDCSSVVDLSPSPPAAVSARKPAAFDLDLNCSPPTEAEA

SEQ ID NO: 251 - protein, 294 aa, Zea mays
MEDVANAHIYAHAHRSKRPQSAAIKDGDGDVDLSMKGARYRGVRRRPWGRFAAEIRDPMSKERRWLGTFDTAEQAACAYDIAARAMRGNKARTNFPGHATAGYWPWGAPQPAAVAHPINPFLLHNLIMSSSNHGCRLLNHAGHGHVHSAAPRPPAPAADATSTTIAAPFPVAAHPAVAMDEDVDDWDGVLRSEPADAGLLQDALHDFYPFTRPRAGGGRRGLSAAGTDARAAAALVAPVKPDAFVVPSPFAGVEGDGEYPMMPQGLLEDVIHSPAFVEVVAAPPSVPTRRGRRG

SEQ ID NO: 252 - protein, 709 aa, Zea mays
MATVNNWLAFSLSPQELPPSQTTDSTLISAATADHVSGDVCFNIPQDWSMRGSELSALVAEPKLEDFLGGISFSEQHHKSNCNLIPSTSSTVCYASSAASTGYHHQLYQPTSSALHFADSVMVASSAGVHDGGSMLSAAAANGVAGAASANGGGIGLSMIKNWLRSQPAPMQPRAAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAATTGLYHPYAQQPMRGGGWCKQEQDHAVIAAAHSLQDLHHLNLGAAGAHDFFSAGQQAAAAAAMHGLASIDSASLEHSTGSNSVVYNGGVGDSNGASAVGSGGGYMMPMSAAGATTTSAMVSHEQMHARAYDEAKQAAQMGYESYLVNAENNGGGRMSAWGTVVSAAAAAAASSNDNIAADVGHGGAQLFSVWNDT

SEQ ID NO: 253 - protein, 492 aa, Zea mays
MDTSHHYHPWLNFSLAHHCDLEEEERGAAAELAAIAGAAPPPKLEDFLGGGVATGGPEAVAPAEMYDSDLKFIAAAGFLGGSAAAAATSPLSSLDQAGSKLALPAAAAAPAPEQRKAVDSFGQRTSIYRGVTRHRWTGRYEAHLWDNSCRREGQSRKGRQVYLGGYDKEEKAARAYDLAALKYWGSSTTTNFPVAEYEKEVEEMKNMTRQEFVASLRRKSSGFSRGASIYRGVTRHHQHGRWQARIGRVAGNKDLYLGTFSTEEEAAEAYDIAAIKFRGLNAVTNFEISRYNVETIMSSNLPVASMSSSAAAAAGGRSSKALESPPSGSLDGGGGMPVVEASTAPPLFIPVKYDQQQQEYLSMLALQQHHQQQQAGNLLQGPLVGFGGLYSSGVNLDFANSHGTAAPSSMAHHCYANGTASASHEHQHQMQQGGENETQPQPQQSSSSCSSLPFATPVAFNGSYESSITAAGPFGYSYPNVAAFQTPIYGME

SEQ ID NO: 254 - protein, 488 aa, Zea mays
MDMDMSSAYPHHWLSFSLSNNYHHGLLEAFSNSSGTPLGDEQGAVEESPRTVEDFLGGVGGAGAPPQPAAAADQDHQLVCGELGSITARFLRHYPAAPAGTTVENPGAVTVAAMSSTDVAGAESDQARRPAETFGQRTSIYRGVTRHRWTGRYEAHLWDNSCRREGQSRKGRQVYLGGYDKEEKAARAYDLAALKYWGPTTTTNFPVSNYEKELEEMKSMTRQEFIASLRRKSSGFSRGASIYRGVTRHHQHGRWQARIGRVAGNKDLYLGTFSTQEEAAEAYDIAAIKFRGLNAVTNFDMSRYDVESILSSDLPVGGGASGRAPAKFPLDSLQPGSAAAMMLAGAAAASQATMPPSEKDYWSLLALHYQQQQEQERQFPASAYEAYGSGGVNVDFTMGTSSGNNNNNTGSGVMWGATTGAVVVGQQDSSGKQGNGYASNIPYAAAAMVSGSAGYEGSTGDNGTWVTTTTSSNTGTAPHYYNYLFGME

SEQ ID NO: 255 - protein, 470 aa, Zea mays
MAHPSAAAAAVSSTAPAANPSSGAREEGGARSPPSPSPSQRGRAKVVIVMGATGAGKSRLAVDLAAHFAGVEVVSADSMQLYRGLDVLTNKAPLHEQNGVPHHLLSVIDPSVEFTCRDFRDRAVPIIQEIVDRGGLPVVVGGTNFYIQALVSPFLLDDMAEEMQGCTLRDHIDDGLTDEDEGNGFERLKEIDPVAAQRIHPNDHRKIKRYLELYATTGALPSDLFQGEAAKKWGRPSNSRLDCCFLWVDADLQVLDSYVNKRVDCMMDGGLLDEVCSIYDADAVYTQGLRQAIGVREFDEFFRAYLPRKESGEGSCASLLGMHDDQLKSLLDEAVSQLKANTRRLVRRQRRRLHRLSKDFGWNLHRVDATEAFFCATDDSWQKKVVKPCVDVVRRFLSDNSTVLPSTSASDPSSRELWTQYVCEACGNRVLRGAHEWEQHRQGRGHRKRVQRLKQKSLRPWPSLLPQDRS

SEQ ID NO: 256 - protein, 359 aa, Zea mays
MEEITQHFGVGASSHGHGHGQHHHHHHHHHPWASSLSAVVAPLPPQPPSAGLPLTLNTVAATGNSGGSGNPVLQLANGGGLLDACVKAKEPSSSSPYAGDVEAIKAKIISHPHYYSLLTAYLECNKVGAPPEVSARLTEIAQEVEARQRTALGGLAAATEPELDQFMEAYHEMLVKFREELTRPLQEAMEFMRRVESQLNSLSISGRSLRNILSSGSSEEDQEGSGGETELPEVDAHGVDQELKHHLLKKYSGYLSSLKQELSKKKKKGKLPKEARQQLLSWWDQHYKWPYPSETQKVALAESTGLDLKQINNWFINQRKRHWKPSEEMHHLMMDGYHTTNAFYMDGHFINDGGLYRLG

SEQ ID NO: 257 - protein, 311 aa, Zea mays
MTGLDEALMLPFTDIDLEAFDNAEEQKPPVDQMVMMPPTVEHPAAAGTRAPIIIDGTATVGQNVGGGVVHAHQKAAMTTIEDSSCFRRGASCVDDDMAVVIHHVERRRQAGSTAVALLPPPQPSLPRPRARASGGAGERSAPAAAGKTRMDHIGFDELRKYFYMPITRAAREMNVGLTVLKKRCRELGVARWPHRKMKSLKSLMANVQEMGNGMSPVAVQHELAALETYCALMEENPWIELTDRTKRLRQACFKESYKRRKAAAGNAIETDHIVYSFGQHRRYKQQLLPPPTAGSTSADDRHGQSSRFFCY

SEQ ID NO: 258 - protein, 391 aa, Zea mays
MAMVPCGGDDAEWCNMMEAINHLMMSSMSSPHVAMGASSCREEDDDSLYLPMYYSSAPPPAVVSDQYCPEQLPPLPAAGAMTGLDEALMLPFTDIDLEAFDNAEEQKPPVDQMVMMPPTVEHPAAAGTRAPIIIDGTATVGQNVGGGVVHAHQKAAMTTIEDSSCFRRGASCVDDDMAVVIHHVERRRQAGSTAVALLPPPQPSLPRPRARASGGAGERSAPAAAGKTRMDHIGFDELRKYFYMPITRAAREMNVGLTVLKKRCRELGVARWPHRKMKSLKSLMANVQEMGNGMSPVAVQHELAALETYCALMEENPWIELTDRTKRLRQACFKESYKRRKAAAGNAIETDHIVYSFGQHRRYKQQLLPPPTAGSTSADDRHGQSSRFFCY

SEQ ID NO: 259 - DNA, 33 bp, herpes simplex
gacgctttggacgacttcgacttggacatgttg

SEQ ID NO: 260 - DNA, 183 bp, herpes simplex
gaagcctctggatctggcagagccgatgccctggatgattttgatctggatatgctgggaagcgacgccctggatgatttcgatctggatatgctgggatctgacgccctggatgatttcgatctggatatgctgggatctgacgccctggatgatttcgatctggacatgctgatcaacagc

SEQ ID NO: 261 - DNA, 1569 bp, Artificial Sequence
  tripartite effector VPR (VP64, p65, and Rta)
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgacatggacttctccgcactgctgggtagcggatcgggatctcgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 262 - DNA, 136 bp, Artificial Sequence
  SAM Part I (modification to the gRNA adding two ms2 hairpin extensions)
gttttagagctaggccaacatgaggatcacccatgtctgcagggcctagcaagttaaaataaggctagtccgttatcaacttggccaacatgaggatcacccatgtctgcagggccaagtggcaccgagtcggtgc

SEQ ID NO: 263 - DNA, 399 bp, Artificial Sequence
  MCP domain
gcggccgctgactacaaggatgacgacgataaatctagaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgaggccagc

SEQ ID NO: 264 - DNA, 56 bp, Artificial Sequence
  Scaffold Part I (modification to the gRNA)
gggagcacatgaggatcacccatgtgcgactcccacagtcactggggagtcttccc

SEQ ID NO: 265 - DNA, 353 bp, Artificial Sequence
  MCP domain
agaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatcta

SEQ ID NO: 266 - DNA, 705 bp, Artificial Sequence
  Suntag Part I (10xGCN4_v4)
gaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaag

SEQ ID NO: 267 - DNA, 831 bp, Artificial Sequence
  Suntag Part II (ScFv_GCN4)
atgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagc

SEQ ID NO: 268 - DNA, 1851 bp, Artificial Sequence
  P300
attttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 269 - DNA, 384 bp, Artificial Sequence
  VP160
gacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttagggtcagacgcactggatgatttcgaccttgatatgttgggaagcgatgcccttgatgatttcgacctggacatgctcggcagcgacgccctggacgatttcgatctggacatgctggggtccgatgccttggatgattttgacttggatatgctggggagtgatgccctggacgactttgacctggacatgctgggctccgatgcgctcgatgacttcgatttggatatgttg

SEQ ID NO: 270 - DNA, 25 bp, Artificial Sequence
  BBM target sequence
tggagtgtaccagttgtataaatat

SEQ ID NO: 271 - DNA, 25 bp, Artificial Sequence
  BBM target sequence
tcctcgaatcattctaagaagaaac

SEQ ID NO: 272 - DNA, 25 bp, Artificial Sequence
  BBM target sequence
tggccgtgacaacgtatactattat

SEQ ID NO: 273 - DNA, 11414 bp, Artificial Sequence
  pGEP362 expression plasmid
agcatgaatgcctgggggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgggtaccgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagccctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacctgcaggcaagca

SEQ ID NO: 274 - DNA, 11414 bp, Artificial Sequence
  pGEP487 expression plasmid
aagcaagcatgaatgcctgggggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgggtaccgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgagggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacctgcaggc

SEQ ID NO: 275 - DNA, 11414 bp, Artificial Sequence
  pGEP488 expression plasmid
cgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacgtagagaccgatcgccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgggtaccgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgc

SEQ ID NO: 276 - DNA, 1572 bp, Artificial Sequence
  VPR transcriptional activation domain
gacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctga

SEQ ID NO: 277 - DNA, 30 bp, Artificial Sequence
  5xGS linker sequence
ggctcggggtcggggtcgggctcgggctcg

SEQ ID NO: 278 - DNA, 4570 bp, Artificial Sequence
  pKWS20 plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 279 - DNA, 13012 bp, Artificial Sequence
  pGEP754 expression plasmid
agcatgaatgcctgggggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgggtaccgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagccctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagca

SEQ ID NO: 280 - DNA, 13013 bp, Artificial Sequence
  pGEP755 expression plasmid
cgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgagggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttaagctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagccctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgggtaccgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgc

SEQ ID NO: 281 - DNA, 13012 bp, Artificial Sequence
  pGEP756 expression plasmid
cgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacgtagagaccgatcgccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagccctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgggtaccgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgc

SEQ ID NO: 282 - DNA, 3768 bp, Lachnospiracea bacterium
atggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgctaggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaag

SEQ ID NO: 283 - DNA, 3768 bp, Artificial Sequence
  LbCpf1_RR
atggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgagggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgctaggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaag

SEQ ID NO: 284 - DNA, 3768 bp, Artificial Sequence
  LbCpf1_RVR
atggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacgtagagaccgatcgccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgctaggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaag

SEQ ID NO: 285 - DNA, 13012 bp, Artificial Sequence
  pGEP767 expression plasmid
agcatgaatgcctgggggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgggtaccgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgctaggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagccctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagca

SEQ ID NO: 286 - DNA, 13013 bp, Artificial Sequence
  pGEP772 expression plasmid
cgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgagggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgctaggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttaagctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagccctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgggtaccgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgc

SEQ ID NO: 287 - DNA, 13012 bp, Artificial Sequence
  pGEP761 expression plasmid
cgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacgtagagaccgatcgccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgctaggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagccctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacaggtcgggtaccgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgc

SEQ ID NO: 288 - DNA, 5370 bp, Artificial Sequence
  dLbCpf1-VPR
atggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgctaggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctga

SEQ ID NO: 289 - DNA, 5370 bp, Artificial Sequence
  LbCpf1(RR)-VPR
atggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgagggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgctaggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctga

SEQ ID NO: 290 - DNA, 5370 bp, Artificial Sequence
  dLbCpf1(RVR)-VPR
atggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacgtagagaccgatcgccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgctaggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggccaagaagaagaagggctcggggtcggggtcgggctcgggctcggacgccctggacgacttcgacctcgacatgctgggctccgacgccctcgatgatttcgacctcgatatgctcggcagcgacgcgctcgatgacttcgacctcgatatgctggggagcgacgccctcgacgattttgacctcgatatgctgatcaactcccgctccagcggcagcccgaagaagaagcgcaaagtgggctcgcagtacctgcccgacaccgacgacaggcacaggatcgaggagaagcgcaagaggacgtacgagaccttcaagtccatcatgaagaagtccccgttcagcggcccaacggacccccgcccgccgccgaggaggatcgccgtgccgtccaggtccagcgcgtcggtccccaagccggccccgcagccctacccgttcacgtccagcctcagcaccatcaactacgacgagttccccaccatggtgttcccgtccggccagatctcccaggccagcgcgctggcccccgcgcccccgcaggtgctgccccaggctccggcccccgctccggccccggccatggtctccgcgctggcccaggcgcccgccccggtgcccgtcctcgcgccgggcccgccgcaggcggtcgccccgccagcgccgaagcccacgcaggccggcgagggcaccctcagcgaggcgctcctgcagctgcagttcgacgacgaggacctcggcgccctcctgggcaactcgaccgaccccgccgtgttcaccgacctggcctccgtcgacaacagcgagttccagcagctgctgaaccagggcatcccggtggcgccgcacaccacggagcccatgctgatggagtacccggaggcgatcacgcgcctcgtcaccggcgcccagaggcccccggaccccgccccggccccgctcggcgccccaggcctgccgaacggcctcctgagcggcgacgaggacttctccagcatcgcggacatggacttctccgccctcctggggtcgggctcgggcagccgcgacagcagggagggcatgttcctcccaaagcccgaggccggctccgccatctcggacgtgttcgagggcagggaggtctgccagccaaagcgcatcaggccgttccacccgccgggctccccgtgggcgaaccggccgctccccgccagcctggctccaaccccgaccggccccgtgcacgagccggtcggcagcctgacgcccgcgccggtgccccagccgctcgaccccgcgccggccgtcacccccgaggcctcccacctcctggaggaccccgacgaggagacctcgcaggccgtgaaggccctgagggagatggccgacaccgtcatcccccagaaggaggaggcggccatctgcggccagatggacctgtcgcacccgccgccgcgcggccacctcgacgagctgaccacgaccctcgagtccatgaccgaggacctcaacctggacagccccctcacgccggagctgaacgagatcctcgacaccttcctgaacgacgagtgcctcctgcacgccatgcacatctccacgggcctgagcatcttcgacaccagcctcttctga

SEQ ID NO: 291 - DNA, 24 bp, Artificial Sequence
  crGEP186 gRNA
gcaagagaggcgaaggagggttcc

SEQ ID NO: 292 - DNA, 24 bp, Artificial Sequence
  crGEP187 gRNA
taaggagggagtgcattggaccta

SEQ ID NO: 293 - DNA, 24 bp, Artificial Sequence
  crGEP188 gRNA
gctctcgctctctgcatgctagct

SEQ ID NO: 294 - DNA, 24 bp, Artificial Sequence
  crGEP201 gRNA
gtatcacccatgggcaatggccat

SEQ ID NO: 295 - DNA, 24 bp, Artificial Sequence
  crGEP208 gRNA
ctcacttcctcgaatcattctaag

SEQ ID NO: 296 - DNA, 24 bp, Artificial Sequence
  crGEP209 gRNA
ctgaataccccaaaactctctgct

SEQ ID NO: 297 - DNA, 24 bp, Artificial Sequence
  crGEP210 gRNA
tgatagcgagatactctatactta

SEQ ID NO: 298 - DNA, 24 bp, Artificial Sequence
  crGEP211 gRNA
gtaagtatagagtatctcgctatc

SEQ ID NO: 299 - DNA, 3841 bp, Artificial Sequence
  crGEP186 expression plasmid
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgcaagagaggcgaaggagggttccggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 300 - DNA, 3841 bp, Artificial Sequence
  crGEP187 expression plasmid
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagattaaggagggagtgcattggacctaggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 301 - DNA, 3841 bp, Artificial Sequence
  crGEP188 expression plasmid
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgctctcgctctctgcatgctagctggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 302 - DNA, 3841 bp, Artificial Sequence
  crGEP201 expression plasmid
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgtatcacccatgggcaatggccatggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 303 - DNA, 3841 bp, Artificial Sequence
  crGEP208 expression plasmid
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatctcacttcctcgaatcattctaagggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 304 - DNA, 3841 bp, Artificial Sequence
  crGEP209 expression plasmid
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatctgaataccccaaaactctctgctggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 305 - DNA, 3841 bp, Artificial Sequence
  crGEP210 expression plasmid
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagattgatagcgagatactctatacttaggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 306 - DNA, 3841 bp, Artificial Sequence
  crGEP211 expression plasmid
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgtaagtatagagtatctcgctatcggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 307 - DNA, 2040 bp, Zea mays; identical to SEQ ID NO: 217

SEQ ID NO: 308 - DNA, 978 bp, Zea mays; identical to SEQ ID NO: 199

SEQ ID NO: 309 - DNA, 1754 bp, Zea mays
atcggacccaaatcatagacacatgatgatataataacagacaaccaaaattgagagtggcaaaatagcaaatttctgatagtcatgtgatagagaatagtagacaattttgacataatatatgtacactaattagtcaacaaaagcgatattgcggttaaaacagtgattgccagtgttttgacccgagtgtcctaaccaaccaataaagtaaatttatgctatgtgtcctcgtccagatggatgatgcaagaagacacaagatttattttggttcggacaatagaaggcctactttcagcggagggggatgggatttatattatcttgcacctaagtgcttgtagtagaaggtacaagttagtcgagagagagagagaatcccaactctctgcggatgattgaggcaagtgtcaatatcggccgcggagggcaataggtgaagtgtattgtcctcctcccttgcaagccttggactccttttatagccttaatgagggaatcaaggagtaataattagttgaagactgattaagaaacagtccatctgttagtttttttgtttaaataggctaaagctaattttatctagttcttaattagctaataattattatttcgtaggatccaaaccattcctaagctatagtgctattatatcaagtgtagatctatatgtactcaaggtcatgatgtttgcaaaccaacaatgaaatttatcgcacacattggtcatggcagatcaacttttttgccacaaaacaaacaagaatagtgcaaacgaagttgcataaaatgaaacaatatattatgtgaatagttgcatggtttatcttgctagttccattttaacacacacacatatcttgctagttccattttaacttctacttgcacaattccaaaaggaacctaaatttcatttaccgatgagtcacaagaaacttagatctaattaaatttaaagaaaaatagcaatatttatatttttaaatatatttattataaaaatttatctcatattctagctaatgatatttattatgcatcataactattaaatatatagctatatatatatatatttcataagtttcatgttgtttaacttaatagagatttatatttttagggctagtttggcaaactatttttccaaaggattttcatttctataaagaaaattatttttttaaaaaaaatagaaatctcttgaaagaatagaattgttaaactactcttagacaaataaagagtatccttggttcgtggctaaccgtatcatattttatctaagttagttgttccaattaaagaactaattttatacacaaaagttaagtaaagtatagcaaattagtccgcgaaccaaatatgaccgaaatatcgaggagtgaggaggcttaacccttcccatgtgtgtatctactgttacaccgtgagctacaaagttactggcacaaacgtatagaggatggtgaggacatgggaagataaaatcctggtccagcaagatccgttcttccaaatgggatcaggtgattggctccagttcctcctcccctcagcaccaccagtctcctccagtccagctcccgtcttctccgcctcaagagtctcagaccaacggcaaagttctagaagcacggttgcacgggcagcacggcataacacctccctccactgatccagttccagtcgcccaacgccccaacgtcttctcttgcaaatcgcaagcaaacttcctgttcacg

SEQ ID NO: 310 - DNA, 658 bp, Zea mays
gttggctacttgagttagattttggttgtgtttcatccccacgtacgtccagcaaagaaaaattgaagctagtgcatgcatggttcgtcatcaaatgcatggccggccggatacaaatttgaactgtagctatcgacgtacgcatgtattaatttatatcagagaagacaaggaacacagatacatacatgtcgaaacaatcattttctatggcacttgagctagctagcatacaattttgttttaaatgaaatgaaactgaagacgatcgatcgaattgaaggttgtggttcgtgagcaatgcaatgcagtttcacagaacgttgccaatgcaacaagccaccaagaaaagagaagtctactcgatcttgcaatgattaggcttggatgatgcgtggggccacgtacgtatggacatcgaagaaccccatcctcagcgtgtggcctgagggtgatggcaaagctgatccacacattgcggccccctttcccccctcagagaccctgacctcccgagcacagccagccaccgcgcaacgccggccaccaccaccaccaccatacctgctagcgctagctctctttatttaacgccgccgtgtgcgtgcctcgacgacctcactactttgagctgcaaggtccgaactaaaaagcaccg

SEQ ID NO: 311 - DNA, 1700 bp, Beta vulgaris
tataagttcaaacttcaatacaggtattttcgggatgtgattaccttacaatttctcattttcaaagaattttacctgtgcagctatgttggataacctgtgcgagattccgtttcagtaggacactttttttttttaccaataaaaaaaaacttataagttcatgagctaatttttatagatagtttaaagtaccgggtggaggatgaatagttgagttttttcttcaaaattagatacttcctccgttttttattagatgttacacttttcaaatcacggactcctaggtaatttttggagaggagagagatagagagaatgaaaaacaaaagggtcccatgtgagtatgtgataggagagagatagagagaatttattacccaaaataaaagtgtaacatctaattcaaaacttcctaaaatagaaagtgtaacatctaaaaaaaacggaggaagtatttgaatttgatatagatattgtgtctttgtgtgtgttgaatttcaattcccagttccctaaaaaaaatttacaattgcaatttcgagattatgatgtaaattaaatttgagagactagaaagtatttggtcaacccaaaaaaaaaatatcaatacttatataaatcaaaaacataatagagaatccaattttactaaaaatattagtaattttgattaaaataatctattaaaatgaactctaaccttcacataatttccacatattattaatcaacaaaataagcatcacaaattattagaataggcgatctaattttaacataaaattagacgaattcaaattgaatttttctaacaagctcattccatttcacgcaacccaaaattatcctagtcagtagtcatccattcttttctcattcctttattcttgattatcgaactacaacagataatttcaaaaaaaaactaaattggtagtcttaactgattaaactacttactaaatggattaaagaatgtcattactgaatagattaaactgattacgaaatagattaacttggtccctaaatagattaaattagttactatattaaaattaggcgatctcttacaaaaccaactgaataagcatagctctgtatattacctagatttcaactaaatcaaaaccccttacagttcaatctagagctgatcattttggctcggcccgtcccatttttgggccgggttttagtcagatttttttggcccgcggtcgggcccggcccgatttttttggctttgggcaagccaaaaacgacttttcagtttattttttggcccgacccgtttttacccgcaaaagcccgctaatttaggtccgcactttgggcacaaaaatttagcccgaacttaaacctggcccgacccatgatcacctctagtttaatccaaactaaaaaactacacaagttagccaaaaattatgtctactttgtacaactttataaaatacacacagtagttgatatcttgatgattaactccttttgaagtttgactacacaccaaccccaaacacacccactttttcccccctcttgtcaccaaccccccctcctctttagccaccaaagtttggttggtgagtcctccataactgctaaattctctcttttttctctctcctaaaaaactaaaacccaccaaaatttcagacatcaaaaaaattacaagtgaaggaaacaat

SEQ ID NO: 312 - DNA, 991 bp, Beta vulgaris
aaagaaggaaaggaaggaatttgaacatgtgacctatcgttcacagcacctcaatcttaatcactagaccaaaacatccttggttcttgcgcaagaaggttggctagaaattttttgtaaaaacactagccccgctcagttcataatgagaatgtcgatgtcaccaaagggatattaaatgaatggaattgggatatggatggaatataatgaaatagagccactttgaggttccctatgaaatgaggcatggaagggagccactacgaaaaagttccgggagttacgaaggaagcttcgagctcatattggtcatgaacccgattactgagtctaataagttcaattgaaaagaaaaagtcttatgttctaaaagaacttttcgtgcggtttgcatgagttcatagtccatataatataatgcaggaatgaagttctcagttgattcttccacacccgtccctcaccccctaggccccaccttcaccccgccgaaaaaaataaagaaaatccaacgttatttttcttagaaatgacagtttgatatagaaaggaaaaataataataaaaaaaaaaagtgttggcgttttcattttcaacctcagtatgttggtttgccccaacaagttctgaaccaattggcgatgtaatcttataagaagaatctaacgttggtccattttgcttctacagttttgaaagttaggtgggccccattattatgttgatcctagaataattaattttggtaggctgagaagaggaaaaataaagaacaatgctaaaaacaagtgaaaaatatagttgcaactcatgatgcaacatgagatgcgatgaaatatgatagtaacttgagctcacaactctgtatataagtgctcatttggacacttattttctacaatttcctagtaactcagcttagcttcattcccgacttttttataaaagtcaggacgatcaatatctatctatttatctgtctgtctgt

SEQ ID NO: 313 - protein, 8 aa, Artificial Sequence
  Cys2His2 motif
  VARIANT (1)..(1): Xaa can be any naturally occurring amino acid
  VARIANT (3)..(3): Xaa can be any naturally occurring amino acid
  VARIANT (5)..(5): Xaa can be any naturally occurring amino acid
  VARIANT (7)..(7): Xaa can be any naturally occurring amino acid
XCXCXHXH

SEQ ID NO: 314 - protein, 9 aa, Artificial Sequence
  LAGLIDADG motif
LAGLIDADG

SEQ ID NO: 315 - DNA, 360 bp, Zea mays
cgaccggatgccgcagccgtagtagagctccttcagcatcctgatctgctccggcgtcggcgtccaccgcgagccgctggggcggcacaccgccggcgcagccacgctgccgctgcctcctcccgctccaccgccgcccgcattggccgccatgcctctatctcagcggccttcctgagcgctcctgtgacctagctctccggtgtccggtctatggcaagagaggcgaaggagggttccttgtttataaggagggagtgcattggacctagaggctagatagctagaaggtagctagcatgcagagagcgagagcgggagaagagagcgtagctgcgctaggtgatataggttggggct

SEQ ID NO: 316 - DNA, 900 bp, Zea mays
taatcgttcttgacagcaacctgccagtcaaatggccgtgacaacgtatactattatcgagtaaaaggtcgccactttagtagtacatgtacatgcatgcgcagatacatcatcaggtactcatatatgggcacacatatagacatgttttgaggaaaatgagacaaagtatagtggagacttccctagaaagcagaagaaaaagaagtggtttatgttccgttaaatcatactacaacttttttttattatactctccattttgtcatcattaggtactcatatatgggcacacatatagtactgccaatttttcttgctaaaaaaagttccactatatatatgtatgtatgcacaaataaactaattttcttagaaaagaaaaccggtgtaatacatactaagggctagtttgggaaccctggtttcctaaggaattttatttttccaaaaaaaatagtttatttttccttcggaaattaggaatctcttataaaattcgagttcccaaactattcctaatatatatatcatactctccatcagtctatatatagattacatatagtaagtatagagtatctcgctatcacatagtgccactaatcttctggagtgtaccagttgtataaatatctatcagtatcagcactactgtttgctgaataccccaaaactctctgcttgacttctcttccctaacctttgcactgtccaaaatggcttcctgatcccctcacttcctcgaatcattctaagaagaaactcaagccgctaccattaggggcagattaattgctgcactttcagataatctaccatggccactgtgaacaactggctcgctttctccctctccccgcaggagctgccgccctcccagacgacggactccacgct

SEQ ID NO: 317 - DNA, 281 bp, Zea mays
atatatagattacatatagtaagtatagagtatctcgctatcacatagtgccactaatcttctggagtgtaccagttgtataaatatctatcagtatcagcactactgtttgctgaataccccaaaactctctgcttgacttctcttccctaacctttgcactgtccaaaatggcttcctgatcccctcacttcctcgaatcattctaagaagaaactcaagccgctaccattaggggcagattaattgctgcactttcagataatctaccatggccactg

SEQ ID NO: 318 - DNA, 372 bp, Zea mays
gatctgctccggcgtcggcgtccaccgcgagccgctggggcggcacaccgccggcgcagccacgctgccgctgcctcctcccgctccaccgccgcccgcattggccgccatgcctctatctcagcggccttcctgagcgctcctgtgacctagctctccggtgtccggtctatggcaagagaggcgaaggagggttccttgtttataaggagggagtgcattggacctagaggctagatagcatgaaggtagctagcatgcagagagcgagagcgggagaagagagcgtagctgcgctaggtgatataggttggggctgggaggggggtcatggccattgcccatgggtgatacgatatcttttggagagag